(19) United States
(12) Patent Application Publication
(10) Pub. No.: US 2026/0217643 A1
(43) Pub. Date: Jul. 30, 2026

(54) SYNTHESIS OF 2-PHENYL-2-AMINO-CYCLOHEXAN-1-ONE DERIVATIVES

(71) Applicant: Gilgamesh Pharmaceuticals, Inc., New York, NY (US)

(72) Inventors: Chris Hugh SENANAYAKE, New York, NY (US); Gopal SIRASANI, New York, NY (US); Bo QU, New York, NY (US); Lalith Palitha SAMANKUMARA, New York, NY (US); Santosh Reddy NAINI, New York, NY (US); Praveen Kumar GAJULA, New York, NY (US); Satya Aravind GANGU, New York, NY (US); William LEONG, New York, NY (US)

(21) Appl. No.: 19/143,441

(22) PCT Filed: Dec. 28, 2023
(86) PCT No.: PCT/US2023/086149

§ 371 Date: Jun. 26, 2025

Related U.S. Application Data
(60) Provisional application No. 63/435,800, filed on Dec. 28, 2022.
(60) Provisional application No. 63/471,091, filed on Jun. 5, 2023.

(51) Int. Cl. C07C 225/20; C07B 59/00; C07C 221/00

(52) U.S. Cl. CPC C07C 225/20; C07B 59/001; C07C 221/00; C07B 2200/05; C07C 2601/14

(57) ABSTRACT

The present disclosure relates to a process for making a compound of the formula I and to the enantioselective synthesis to prepare the S or R isomers. It also relates to intermediates in the process.

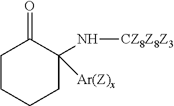

### CROSS-REFERENCE TO RELATED APPLICATIONS

The present application claims benefit of U.S. Ser. No. 63/435,800, filed on Dec. 28, 2022, and U.S. Ser. No. 63/471,091 filed on Jun. 5, 2023, the contents of both of which are incorporated by reference.

### FIELD OF THE DISCLOSURE

The present disclosure relates to novel syntheses of 2-aminocyclohexan-1-one derivatives which are useful in the treatment of psychiatric disorders.

### BACKGROUND OF THE DISCLOSURE

U.S. Pat. No. 11,344,510, the contents of which are incorporated by reference, discloses, in part, compounds useful for the treatment of psychiatric disorders having the following formula:

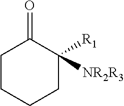

or pharmaceutically acceptable salts,
wherein R₁ is selected from the group consisting of phenyl, optionally substituted thiazole, optionally substituted thiophene, optionally substituted pyridine, a moiety of the following general formula:

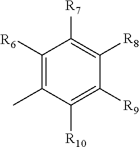

wherein when R₁ is phenyl then R₂ and R₃ are independently selected from H, CD₃, branched or cyclo C3 alkyl, C4-C10 alkyl, C2-C10 halo-alkyl, —R₄—O—R₅; wherein R₄ is a C2-C10 alkylene and R₅ is selected from H and C1-C10 alkyl; wherein D represents a deuterium-enriched —H site; provided that one or more of R₂ and R₃ is different than H; or
R₂ and R₃ are independently selected from C2-C10 alkyl; C2-C10 halo-alkyl, —R₄—O—R₅; wherein R₄ is a C2-C10 alkylene and R₅ is selected from H and C1-C10 alkyl; or R₂ and R₃ together with the nitrogen atom they are connected to form a C3-C9 cycloheteroalkyl ring; said ring optionally substituted by one or more C1-C10 alkyl or interrupted by one or more additional nitrogen or oxygen atoms;
wherein when R₁ is a moiety of general formula:

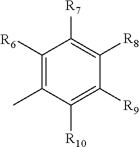

R₂ and R₃ are independently selected from H, C1-C10 alkyl, C2-C10 halo-alkyl, —R₄—O—R₅; wherein R₄ is a C2-C10 alkylene and R₅ is selected from H and C1-C10 alkyl; provided that one or more of R₂ and R₃ is different than H; or
R₂ and R₃ together with the nitrogen atom they are connected to form a C3-C9 cycloheteroalkyl ring; said ring optionally substituted by one or more C1-C10 alkyl or interrupted by one or more additional nitrogen or oxygen atoms; and wherein R₆, R₇, R₈, R₉ and R₁₀ are independently selected from H, OH, halogen (selected from F, Cl, Br, I), —OR11, C1-C10 alkyl, C2-C10 alkenyl, C2-C10 alkynyl, CN, CF₃, OCF₃, NO₂, —NR12R13, —SR14, —SO₂R15, —CO₂R16, —C(═O)NR17R18;
wherein R11, R12, R13, R14, R15, R16, R17 and R18 are each independently selected from H, C1-C10 alkyl, C2-C10 alkenyl, C2-C10 alkynyl, aryl, heteroaryl, —C(═O)H, —C(═O)alkyl, —C(═O)aryl, —C(═O)heteroaryl;
   provided that one or more of R₆-R₁₀ is different than H; or provided that when R₆ is Cl and R₇-R₁₀ are H or when R₇ is Cl and R₆, R₈-R₁₀ are H, and R₂ or R₃ is H, then the other of R₂ or R₃ is C3-C10 alkyl, C2-C10 halo-alkyl, or —R₄—O—R₅; or
provided that when R₇ is OH and R₆, R₈-R₁₀ are H, and R₂ or R₃ is H, then the other of R₂ or R₃ is straight or branched C3-C10 alkyl, C2-C10 halo-alkyl, or —R₄—O—R₅; or
provided that when R₆, R₇, or R₈ is OMe and the other of R₆-R₁₀ are each H, and R₂ or R₃ is H, then the other of R₂ or R₃ is C3-C10 alkyl, C2-C10 halo-alkyl, or —R₄—O—R₅;
wherein when R₁ is selected from thiazole, thiophene, pyridine, each optionally substituted with one or more OH, halogen (selected from F, Cl, Br, I), —OR19, C1-C10 alkyl, C2-C10 alkenyl, C2-C10 alkynyl, CN, CF₃, OCF₃, NO₂, —NR20R21, —SR22, —SO₂R23, —CO₂R24, —C(═O)NR25R26; wherein R19, R20, R21, R22, R23, R24, R25 and R26 are each independently selected from H, C1-C10 alkyl, C2-C10 alkenyl, C2-C10 alkynyl, aryl, heteroaryl, C(═O)H, C(═O)alkyl, C(═O)aryl, C(═O)heteroaryl; R₂ and R₃ are independently selected from H, C1-C10 alkyl, C2-C10 halo-alkyl, C2-C10 alkenyl, C2-C10 alkynyl, —R₄—O—R₅; wherein R₄ is C2-C10 alkylene and R₅ is selected from H and C1-C10 alkyl; or R₂ and R₃ together with the nitrogen atom they are connected to form a C3-C9 cycloheteroalkyl ring; said ring optionally substituted by one or more C1-C10 alkyl or interrupted by one or more additional nitrogen or oxygen atoms.

It also discloses, in part, compositions comprising a compound having the general structure (A) useful for the treatment of psychiatric disorders:

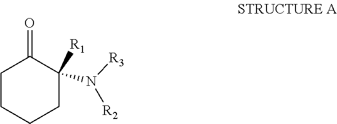

wherein R₁ is selected from the group consisting of phenyl, optionally substituted thiazole, optionally substituted thiophene, optionally substituted pyridine, a moiety of general formula (B);
   wherein when R₁ is phenyl;
then R₂ and R₃ are independently selected from H, CD₃, branched or cyclo C3 alkyl, C4-C10 alkyl, C2-C10 halo-alkyl, —R₄—O—R₅; wherein R₄ is a C2-C10 alkylene and R₅ is selected from H and C1-C10 alkyl; wherein D represents a deuterium-enriched —H site; and wherein at least one of R₂ and R₃ is other than H; or
R₂ and R₃ are independently selected from C2-C10 alkyl; C2-C10 halo-alkyl, —R₄—O—R₅; wherein R₄ is a C2-C10 alkylene and R₅ is selected from H and C1-C10 alkyl; or
R₂ and R₃ together with the nitrogen atom they are connected to form a C3-C9 cycloheteroalkyl ring, said ring optionally substituted by one or more C1-C10 alkyl or interrupted by one or more additional nitrogen or oxygen atoms;
wherein when R₁ is a moiety of general structure (B):

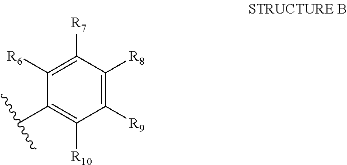

R₂ and R₃ are independently selected from H, C1-C10 alkyl, C2-C10 halo-alkyl, —R₄—O—R₅; wherein R₄ is a C2-C10 alkylene and R₅ is selected from H and C1-C10 alkyl; and wherein at least one of R₂ and R₃ is other than H; or
R₂ and R₃ together with the nitrogen atom they are connected to form a C3-C9 cycloheteroalkyl ring; said ring optionally substituted by one or more C1-C10 alkyl or interrupted by one or more additional nitrogen or oxygen atoms; and
R₆, R₇, R₈, R₉ and R₁₀ are independently selected from H, OH, halogen (selected from F, Cl, Br, I), —OR11, C1-C10 alkyl, C2-C10 alkenyl, C2-C10 alkynyl, CN, CF₃, OCF₃, NO₂, —NR12R13, —SR14, —SO₂R15, —CO₂R16, —C(═O)NR17R18; wherein R11, R12, R13, R14, R15, R16, R17 and R18 are each independently selected from H, C1-C10 alkyl, C2-C10 alkenyl, C2-C10 alkynyl, aryl, heteroaryl, —C(═O)H, —C(═O)alkyl, —C(═O)aryl, —C(═O)heteroaryl; wherein at least one of R₆-R₁₀ is other than H; and wherein neither R₆ nor R₁₀ is halogen; provided that when R₇ is Cl and R₆, R₈-R₁₀ are H, and R₂ or R₃ is H, then the other of R₂ or R₃ is C3-C10 alkyl, C2-C10 halo-alkyl, or —R₄—O—R₅; or
provided that when R₇ is OH and R₆, R₈-R₁₀ are H, and R₂ or R₃ is H, then the other of R₂ or R₃ is C3-C10 alkyl, C2-C10 halo-alkyl, or —R₄—O—R₅; or
provided that when R₆, R₇, or R₈ is OMe and the other of R₆-R₁₀ are each H, and R₂ or R₃ is H, then the other of R₂ or R₃ is C3-C10 alkyl, C2-C10 halo-alkyl, or —R4-O—R₅;
wherein when R₁ is selected from thiazole, thiophene, and pyridine, each optionally substituted with one or more OH, halogen (selected from F, Cl, Br, I), —OR19, C1-C10 alkyl, C2-C10 alkenyl, C2-C10 alkynyl, CN, CF₃, OCF₃, NO₂, —NR20R21, —SR22, —SO₂R₂₃, —CO₂R₂₄, —C(═O)NR25R26; wherein R19, R20, R21, R22, R23, R24, R25 and R26 are each independently selected from H, C1-C10 alkyl, C2-C10 alkenyl, C2-C10 alkynyl, aryl, heteroaryl, C(═O)H, C(═O)alkyl, C(═O)aryl, C(═O)heteroaryl;
R₂ and R₃ are independently selected from H, C1-C10 alkyl, C2-C10 halo-alkyl, C2-C10 alkenyl, C2-C10 alkynyl, —R₄—O—R₅; wherein R₄ is a C2-C10 alkylene and R₅ is selected from H and C1-C10 alkyl; or
R₂ and R₃ together with the nitrogen atom they are connected to form a C3-C9 cycloheteroalkyl ring; said ring optionally substituted by one or more C1-C10 alkyl or interrupted by one or more additional nitrogen or oxygen atoms;
or a pharmaceutically acceptable salt or ester of the compound, wherein the composition is enriched in the compound over its opposite enantiomer.

The '510 patent discloses methods for preparation of compounds disclosed therein. The present disclosure discloses different, efficient methods for preparing compounds disclosed therein. The present disclosure provides compounds useful for the treatment of psychiatric disorders, for example, 2-(4-fluorophenyl)-2-(methylamino)cyclohan-1-one hydrochloride and stereoisomers of same, e.g., (R)-2-(4-fluorophenyl)-2-(methylamino)cyclohan-1-one hydrochloride and (S)-2-(4-fluorophenyl)-2-(methylamino)cyclohan-1-one hydrochloride.

### SUMMARY OF THE DISCLOSURE

The present disclosure relates to a process for preparing the compound of formula I:

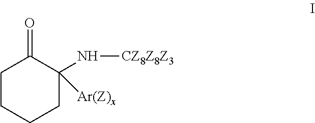

which process comprises
(a) reacting a compound having a ketal protecting group of the formula

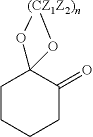

with a sulfinamide of the formula

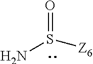

in the presence of Ti(OZ₇)a(X₁)_{b} to form an imine of Formula V

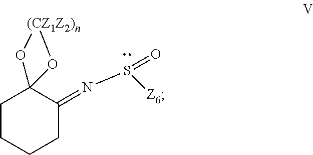

(b) reacting the imine of Formula V with an aryl nucleophilic agent Nu, capable of delivering an aryl nucleophilic moiety of the formula Ar(Z)ₓ under arylation reaction conditions to form a sulfinamide of Formula VII

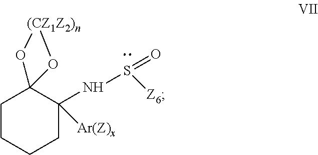

(c) reacting the sulfinamide of Formula VII with acid to form the corresponding amine of Formula VIII

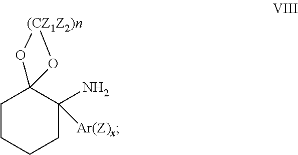

(d) reacting the amine of Formula VIII with an acylating agent of formula Z₃COOXₒ or acid derivative thereof under amide forming conditions to form the amide of Formula IX having an acyl group C(═O)Z₃

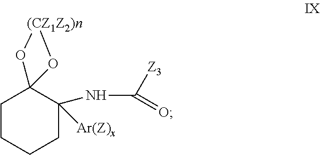

(e) reducing the acyl group in Formula IX with an acyl reducing agent under acyl reducing conditions to form the compound of Formula X

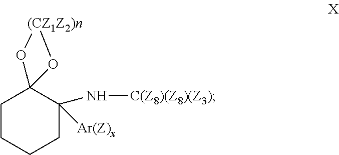

(f) deprotecting the ketal in the presence of acid to form a compound of formula I;
wherein
each Z₁ is independently H or C1-C6 alkyl;
each Z₂ is independently H or C1-C6 alkyl;
n is 2 or 3;
Z₃ is hydrogen, C1-C6 alkyl optionally substituted with one or more halogen, C1-C6 alkoxy, or aryl optionally substituted with one or more C1-C6 alkyl, C1-C6 alkoxy or halogen or Z₃ is D;
each Z is independently halogen (selected from F, Cl, Br, I), —OZ₁₁, C1-C10 alkyl, C2-C10 alkenyl, C3-C10 alkynyl having no terminal hydrogen atoms, e.g., C4-C10 alkynyl having no terminal hydrogen atoms, CF₃, or OCF₃; wherein each Z₁₁ is independently selected from C1-C10 alkyl, C2-C10 alkenyl, C3-C10 alkynyl having no terminal hydrogen atoms, and aryl which is unsubstituted or substituted with C1-C6 alkyl, C1-C6 alkoxy or halogen;
Z₇ is C1-C4 alkyl;
each Z₈ are independently H or D;
Z₆ is C1-C6 alkyl, C3-C10 cycloalkyl or hydrocarbyl aryl or hydrocarbyl ar(C1-C3) alkyl, each of may be unsubstituted or substituted with one or more halo, or C1-C6 alkyl or C1-C6 alkoxy;
X₁ is halo;
Xₒ is H or D;
a is 0, 1, 2, 3, or 4;
b is 0, 1, 2, 3 or 4;
a+b=4;
x is 0, 1, 2, 3, 4, or 5; provided that when Ar is thiophene, x is 0, 1, 2, or 3; Ar is aryl; and

Nu is an aryl nucleophilic agent capable of delivering an aryl nucleophilic moiety of the formula Ar(Z)ₓ, provided that when Z₃ and each Z₈ are D, then the acyl reducing agent is deuterated and acyl group of formula Z₃COOXₒ or acid derivative thereof is deuterated.

In another embodiment, Z₆ is C1-C6 alkyl or phenyl, which is unsubstituted or substituted with C1-C6 alkyl; Nu is an organometallic compound M, in which the reaction between the imine and M is effected under organometallic reaction conditions to form the sulfonamide of Formula VII; and M is Ar(Z)xMgX or Ar(Z)Li, and Z₁, Z₂, n, Z₃, Z, Z₁₁, Z₇, Z₈, X₁, Xₒ, a, b, x, and Ar are as defined hereinabove. In an embodiment, Z is halo and x is 1 or 2, and in another embodiment, Z is F and x is 1 or 2, for example, x is 1. In a further embodiment, F is on the 4-position (para position on the phenyl ring.

In another embodiment, the present disclosure relate to a process of preparing the compound of Formula I herein, comprising
(a) reacting a compound having a ketal protecting group of the formula

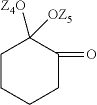

with a sulfinamide of the formula

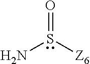

in the presence of Ti(OZ₇)ₐX_{1b} to form an imine of Formula VA

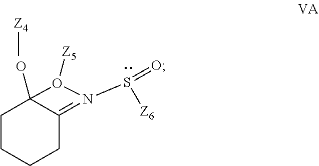

(b) reacting the imine of Formula VA with an aryl nucleophilic agent Nu, capable of delivering an aryl nucleophilic moiety of the formula Ar(Z)ₓ under arylation reaction conditions to form a sulfinamide of Formula VIIA

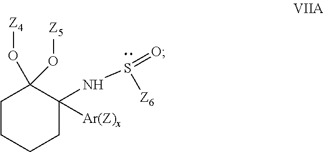

(c) reacting the sulfinamide of Formula VIIA with acid to form the corresponding amine of Formula VIIIA

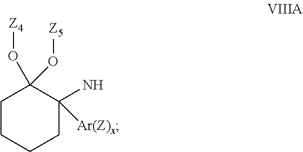

(d) reacting the amine of Formula VIIIA with an acylating agent of formula Z₃COOXₒ or acid derivative thereof to form the amide of Formula IXA having an acyl group (C(═O)Z₃

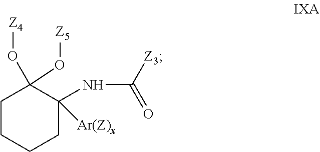

(e) reducing the acyl group in formula IXA with an acyl reducing agent to form the compound of Formula XA

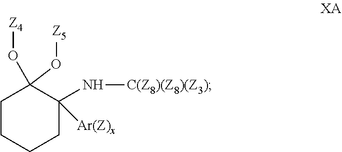

(f) deprotecting the ketal to form a compound of Formula I;
wherein
Z₄ is C1-C6 alkyl;
Z₅ is C1-C6 alkyl;
Z₃ is H, C1-C6 alkyl optionally substituted with one or more halogen or C1-C6 alkoxy, or aryl optionally substituted with one or more C1-C6 alkyl, C1-C6 alkoxy, or halogen;
each Z is independently halogen (selected from F, Cl, Br, I), —OR₁₁, C1-C10 alkyl, C2-C10 alkenyl, C3-C10 alkynyl having no terminal hydrogen atoms, e.g., C4-C10 alkynyl having no terminal hydrogen atoms, CF₃, or OCF₃; wherein each Z₁₁ is independently selected from C1-C10 alkyl, C2-C10 alkenyl, C3-C10 alkynyl having no terminal hydrogen atoms, e.g., C4 —C10 alkynyl having no terminal hydrogen atoms, and aryl which is unsubstituted or substituted with C1-C6 alkyl, C1-C6 alkoxy, or halogen;
Z₆ is C1-C6 alkyl, C3-C10 cycloalkyl, hydrocarbyl aryl, or hydrocarbyl ar(C1-C3) alkyl, each of which may be unsubstituted or substituted with one or more halo or C1-C6 alkyl or C1-C6 alkoxy;
Z₇ is C1-C4 alkyl;
Each Z₈ is independently H or D;
X₁ is halo;
Xₒ is H or D;
a is 0, 1, 2, 3, or 4;
b is 0, 1, 2, 3, or 4;
a+b=4
x is 0, 1, 2, 3, 4, or 5; and
Ar is aryl;
provided that when Ar is thiophene, x is 0, 1, 2, or 3, and provided that when Z₃ and each Z₈ are D, then the acyl reducing agent is deuterated and acyl group of formula Z₃COOXₒ or acid derivative thereof is deuterated.

In another embodiment, Z₆ is C1-C6 alkyl or phenyl, which is unsubstituted or substituted with C1-C6 alkyl; Nu is an organometallic compound M, in which the reaction between the imine and M is effected under organometallic reaction conditions to form the sulfonamide of Formula VII; and M is Ar(Z)xMgX or Ar(Z)Li, and Z₄, Z₅, n, Z₃, Z, Z₁₁, Z₇, Z₈, X₁, Xₒ, a, b, x, and Ar are as defined hereinabove. In an embodiment, Z is halo and x is 1 or 2, and in another embodiment, Z is F and x is 1 or 2, for example, x is 1. In a further embodiment, F is on the 4-position (para position on the phenyl ring.

Another aspect of the present disclosure is directed to a process for preparing a compound of Formula I wherein Ar is phenyl substituted with fluoro

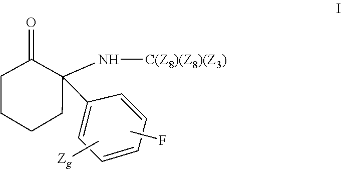

which process comprises
   (a) reacting a compound having a ketal protecting group of the formula

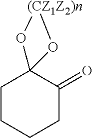

with t-butylsulfinamide in the presence of Ti(OEt)₄ to form an imine of Formula V;

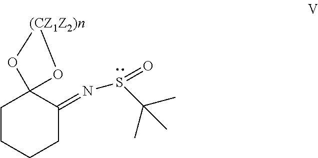

(b) reacting the imine of Formula V with a Grignard reagent of the formula:

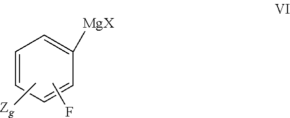

under Grignard reaction conditions to form a sulfinamide product of Formula VII:

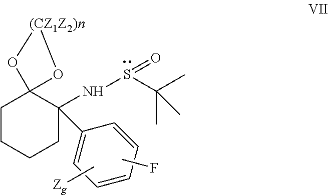

(c) reacting the compound of Formula VII with acid to form an amine of Formula VIII:

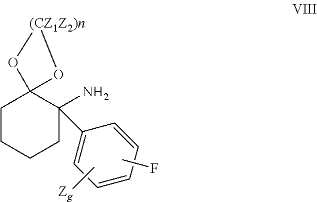

(d) reacting the amine of Formula VIII with an acylating agent of the formula Z₃COOXₒ or acid derivative thereof to form the amide under amide forming conditions of Formula IX having an acyl group C(═O)Z₃

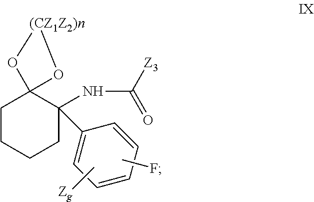

(e) reducing the acyl group in formula IX with an acyl reducing agent under acyl reducing conditions to form the compound of Formula X:

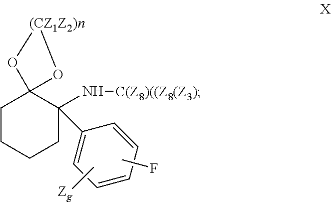

and
(f) deprotecting the ketal to form the compound of Formula I;
wherein
each Z₁ is independently H or C1-C6 alkyl;
each Z₂ is independently H or C1-C6 alkyl;
n is 2 or 3;
Z₃ is hydrogen, C1-C6 alkyl optionally substituted with one or more halogen, C1-C6 alkoxy, C1-C6 alkyl, or aryl optionally substituted with C1-C6 alkyl, C1-C6 alkoxy or halogen or Z₃ is D;
each Z is independently halogen (selected from F, Cl, Br, I), —OZ₁₁, C1-C10 alkyl, C2-C10 alkenyl, C4-C10 alkynyl having no terminal hydrogen atoms, CF₃, or OCF₃; wherein each Z₁₁ is independently selected from C1-C10 alkyl, C2-C10 alkenyl, C3-C10 alkynyl having no terminal hydrogen atoms, e.g., C4-C10 alkynyl having no terminal hydrogen atoms, and aryl which is unsubstituted or substituted with C1-C6 alkyl, C1-C6 alkoxy or halogen,
each Z₈ is independently H or D;
X is halo;
Xₒ is H or D; and
g is 0, 1, 2, 3, or 4;
provided that when Z₃ and each Z₈ are D, then the acyl reducing agent is deuterated and acyl group of formula Z₃COOXₒ or acid derivative thereof is deuterated. In an embodiment, g is 0 or 1 or 2, and in another embodiment, g is 0 or 1 and in a further embodiment, g is 0, that is, Z_{g} is H.

In another embodiment, the present disclosure relates to the syntheses of the corresponding enantiomeric compounds of Formula I, that is the R and S isomers, as described hereinbelow. Further, the present disclosure also relates to novel intermediates, both the racemic compounds as well as the R and S stereoisomers, as described hereinbelow.

In another embodiment, the present disclosure relates to the process of making pharmaceutically acceptable salts of Formula I and/or its R and S stereoisomers by deprotecting the ketal of the formula:

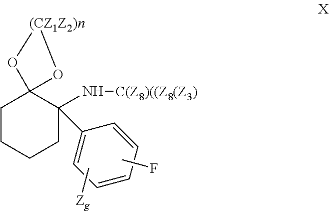

or its R or S stereoisomers, respectively in the presence of acid or by deprotecting the ketal group of Formula X or its R or S stereoisomer, respectively first and then treating the deprotected product with acid, wherein Z₁, Z₂, each Z₈, Z₃, Z, n and g are as defined herein.

In another embodiment, the present disclosure relates to a compound having the following formula:

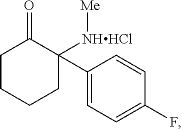

pharmaceutical compositions comprising same and methods for the treatment of psychiatric disorders. In another embodiment, the present disclosure relates to the above compound in solid form. In a further embodiment, the present specification relates to the R isomer of the hydrochloride salt described hereinabove having the formula:

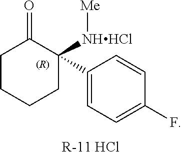

In another embodiment, the present disclosure relates to the compound R-11-HCl in solid form

In a still further embodiment, the present disclosure relates to a compound having the formula:

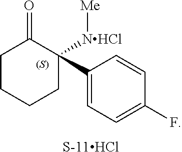

In an even further embodiment, the present disclosure relates to a compound of the formula:

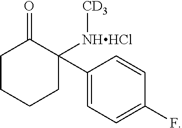

In another embodiment, the present disclosure relates to a compound of the formula:

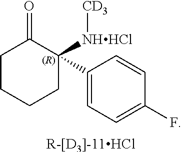

while in another embodiment, the present disclosure relates to a compound of the formula:

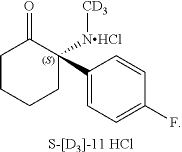

### DETAILED DESCRIPTION

The features and other details of the disclosure will now be more particularly described. Before further description of the present disclosure, certain terms employed in the specification, examples, and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

Compounds disclosed herein may include at least one asymmetric center. When the stereoisomers are specifically designated, these centers are specifically indicated by the symbols “R” or “S,” depending on the configuration of substituents around the chiral atom. However, when stereochemistry is not to be designated, the structures will be drawn without indicating the stereochemistry; these structures are racemic mixtures. Unless otherwise indicated in the structural formula, it should be understood that the present disclosure encompasses all possible stereochemical isomeric forms, including diastereomeric, enantiomeric, and epimeric forms, as well as d-isomers and 1-isomers, and mixtures thereof, wherever possible. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure contemplates all cis, trans, syn, anti, entgegen (E), and zusammen (Z) isomers as well as the appropriate mixtures thereof. Additionally, compounds may exist as tautomers; all tautomeric isomers are provided by the present disclosure. Additionally, the compounds disclosed herein can exist in unsolvated as well as solvated forms with pharmaceutically acceptable solvents such as water, ethanol, and the like; and mixture of solvents thereof. In general, unless indicated to the contrary, the solvated forms are considered equivalent to the unsolvated forms.

The compounds of general formula without stereochemical designation will be depicted by a Roman numeral, such as I, II, III, IV, V, X, and the like. Compounds with Roman numerals also depict compounds with no stereochemical designation having a cyclic ketal as a protecting group. When the protecting group for the carbonyl is a noncyclic ketal, it is designated with an “A.” For example, an open chain ketal as the protecting compound for a carbonyl moiety in the molecule, will be designated, for instance, as VA, XA, and the like. If stereochemistry is to be designated, the compound will be depicted with a letter R or S, depending on whether the compound is a R stereoisomer or S stereoisomer, depending upon the configuration around the asymmetric carbon present therein. For example, a compound of formula IR refers to the compound of Formula I in the R configuration at the asymmetric carbon atom present therein and a compound of Formula IS refers to the compound of Formula I in the S configuration at the asymmetric carbon atom present therein. As another example, the compound designated as compound VAS refers to a compound of formula VA having a non-cyclic ketal in the S configuration.

In some embodiments, a composition prepared herein may be enriched in a specific enantiomer of any compound disclosed herein relative to the corresponding opposite enantiomer of that compound, such that the mixture is not racemic. In such cases, the subject mixture of isomers is understood to have an enantiomeric excess and optical purity >0%. The enantiomeric excess or optical purity of the isomeric mixture may be, for example, >0%, >5%, >25%, >50%, >75%, >90%, >95%, >97%, >98%, or >99%. The enantiomeric excess or optical purity of the isomeric mixture may be, for example, 5-100%, 25-100%, 50-100%, 75-100%, 90-100%, 95-100%, 97-100%, 98-100%, or 99-100%. Thus, for example, contemplated herein is a composition including the S enantiomer of a compound substantially free of the R enantiomer, or the R enantiomer substantially free of the S enantiomer. Further, if the named compound includes more than one chiral center, the scope of the present disclosure also includes compositions containing the various stereoisomers and diastereomers, including mixtures of varying proportions between the various stereoisomers and/or diastereomers or pharmaceutically acceptable salts thereof, as well as compositions including one or more stereoisomers and diastereomers substantially free of one or more of the other stereoisomers and/or diastereomers, respectively. By “substantially free” it is meant that the composition includes less than, for example, 50%, 25%, 15%, 10%, 8%, 5%, 3%, 2%, or 1% of the minor enantiomer or diastereomer(s). For example, the expression that a compound is enantiomerically pure refers to the compound being substantially free of other stereoisomers, including any other enantiomers or diastereomers.

For clarity, in the context of the present disclosure, chemical structures of a compound depicted with a specific stereochemical orientation at any particular chiral center, as defined by wedge and dash notation, are intended to represent the specified stereoisomer of said compound in substantially pure form, or a mixture enriched in the stereoisomer(s) with the specified stereochemical orientation at the defined chiral center over the stereoisomer(s) with the opposite orientation at said chiral center.

Pharmaceutically acceptable salts,” as used herein, refers to any salt of a compound of Formula I or variations thereof, such as I, IR, or IS, and the like, disclosed herein above and below, including any pharmaceutically acceptable salt, wherein the aforementioned compounds are basic in nature and an acidic compound is added thereto to form said salt. The phrase “pharmaceutically acceptable salt(s)”, as used herein, means those salts of the aforementioned compounds of Formula I or variations thereof, such as IR or IS, and the like, disclosed herein that are safe and effective for pharmaceutical use in mammals and that possess the desired biological activity. Pharmaceutically acceptable salts include salts of basic groups present in compounds of Formula I or variations thereof, such as IR or IS, and the like, disclosed herein. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds disclosed herein can form pharmaceutically acceptable salts with various amino acids. For a review on pharmaceutically acceptable salts see BERGE Et. Al., 66 J. PHARM. SCI. 1-19 (1977), and Handbook of Pharmaceutical Salts: Properties, Selection, and Use, edited by P. Heinrich Stahl and Camile G. Wermuth. VHCA, Verlag Helvetica Chimica Acta, Zurich, Switzerland, and Wiley-VCH, Weinheim, Germany. 2002, pp. vix-374 (ISBN 3-906390-26-8), the contents of both of which are incorporated herein by reference.

The present disclosure is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include ¹³C and ¹⁴C.

It will be noted that any notation of a carbon in structures throughout this application, when used without further notation, are intended to represent all isotopes of carbon, such as ¹²C, ¹³C or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In some embodiments, each D in a chemical structure represents a deuterium-enriched —H site, and the level of deuterium-enriched-H site of the compound is 20-100%, 50-100%, 70-100%, 90-100%, 95-100%, 97-100% or 99-100%.

It is understood that substituents and substitution patterns on the compounds used in the method of the present disclosure can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present disclosure, one of ordinary skill in the art will recognize that the various substituents, i.e., Z₁, Z₂, and the like are to be chosen in conformity with well-known principles of chemical structure connectivity.

The terms “about” or “approximately” as used herein is defined as being within an acceptable error range for a particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, a range up to 10%, a range up to 5%, and/or a range up to 1% of a given value. About” and “approximately” are used interchangeably herein.

The term “Nu”, as used herein, refers to a “nucleophile.” As used herein, the term Nu refers to an aryl nucleophile having the moiety Ar(Z)ₓ as the nucleophile, wherein Ar is phenyl, and Z and x are as defined herein, that is capable of delivering an aryl nucleophilic moiety of the formula Ar(Z)ₓ. Examples include molecules in which the moiety Ar(Z)ₓ is present in an organometallic, such as a organomagnesium compound, such as a Grignard reagent, for example, MgBrAr(Z)ₓ, or organolithium compounds, e.g., LiAr(Z)ₓ, or organo-potassium, sodium, or aluminum, such as organoaluminates, organocopper, zinc, tin, and the like or is present in a non-metallic compound, such as an organoborate, wherein the moiety Ar(Z)ₓ is bonded to a boron atom or arylphosphonium salt such as a tetraarylphosphonium salt having the moiety [Ar(Z)ₓ]₄ P⁺, and the like. When describing a reaction herein in which the Ar(Z)ₓ is an aryl nucleophile, the term “Nu” will be used to represent the molecule in which the Ar(Z)ₓ is present as an aryl nucleophile.

The term “alkyl” as used herein, unless indicated to the contrary, refers to a saturated straight or branched hydrocarbon, having the number of carbon atoms specified herein, for example, 1 to 6 carbon atoms. Exemplary alkyl groups include, but are not limited to, straight or branched hydrocarbons of 1-6, 1-4, or 1-3 carbon atoms, referred to herein as C1-C6 alkyl, C1-C4 alkyl, and C1-C3 alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, and the like.

The term “alkenyl” as used herein is a branched or unbranched hydrocarbon group having a specified number of carbon atoms and containing at least one double bond as defined hereinbelow, for example, having 2-6 carbon atoms and 1-3 carbon-carbon double bonds. In some embodiments, alkenyl refers to a branched or unbranched unsaturated hydrocarbon group having three carbon atoms (C₃). In some embodiments, alkenyl refers to a branched or unbranched hydrocarbon group having six carbon atoms (C₆). In some embodiments, the term “alkenyl” includes, but is not limited to, vinyl or allyl, 2-methyl-1-pentenyl, 2-propenyl, 1-propenyl, and the like.

The term “alkynyl” as used herein is a branched or unbranched hydrocarbon group having a specified number of carbon atoms and containing at least one triple bond as described hereinbelow, for example, having 4-6 carbon atoms, and 1-3 carbon-carbon triple bonds, and do not have any terminal hydrogen. In some embodiments, alkynyl refers to a branched or unbranched unsaturated hydrocarbon group having four carbon atoms (C₄) without any terminal hydrogen atoms. In some embodiments, alkynyl refers to a branched or unbranched hydrocarbon group having six carbon atoms (C₆) without any terminal hydrogen atoms. Example includes 2-butynyl, 3-pentynyl, 2-penenyyl, 2-ethyl-2-butynyl, and the like.

The term “alkyne having no terminal hydrogen atoms” or similar term is a term of art and is understood by one of ordinary skill in the art. As understood by one of ordinary skill in the art, it refer to an alkyne which does not have a —C≡CH moiety.

The term “cyano” as used herein refers to the radical —CN.

The terms “cycloalkyl” or a “carbocyclic group” as used herein, refers to a non-aromatic ring system wherein the ring atoms are only carbon atoms containing 3-14 ring carbon atoms. These terms include a saturated or partially unsaturated cyclic hydrocarbon group of, for example, 3-6, or 4-6 carbons, referred to herein as C3-C6 cycloalkyl or C4-C6 cycloalkyl, respectively. Exemplary cycloalkyl groups include, but are not limited to, cyclohexyl, cyclopentyl, cyclopentenyl, cyclobutyl, cyclopropyl, cyclooctyl, decalinyl, and the like.

The terms “halo” or “halogen” or “halide” as used herein refer to F, Cl, Br, or I.

The term “acid derivative of Z₃COOXₒ” refers to an acid halide, ester, or anhydride thereof.

The term “ester” refers to a moiety of formula Z₃COOZ₁₂ wherein Z₃ is as defined herein, wherein Z₁₂ is C1-C6 alkyl or aryl which alkyl groups and aryl groups are unsubstituted or are substituted with halogen, or C1-C6 alkoxy, or arylC₁-C6alkyl, C1-C6 alkyl, aryl and the like.

The term “anhydride” refers to a compound of the formula Z₃COOCOZ₁₂, wherein Z₃ and Z₁₂ are as defined herein. In an embodiment, Z₁₂ has the same definition as Z₃.

The term “aryl” used alone or as part of a larger moiety as in “arylalkyl”, “arylalkoxy”, or “aryloxyalkyl”, refers to monocyclic and bicyclic ring systems having a total of five to fourteen ring atoms, wherein at least one ring in the system is aromatic and wherein each ring in the system contains three to seven ring atoms. The term “aryl” may be used interchangeably with the term “aryl ring”. In certain embodiments of the present disclosure, “aryl” refers to an aromatic ring system which includes, but is not limited to, phenyl, biphenyl, naphthyl, anthracenyl and the like, which may bear one or more substituents which are not reactive with a Grignard reagent. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like. In addition, aryl refers to thiophene and benzothiophenes.

In the context of the present disclosure, the term “thiophene” should be understood to refer to a moiety having the structure

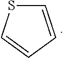

Moreover, the term “benzothiophene” refers to compounds of the formula

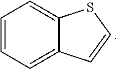

The term “hydrocarbyl aryl” or synonym thereto refers to an aromatic monocyclic or bicyclic ring system having a total of five to 14 carbon ring atoms in the aromatic ring, wherein at least one ring in the system is aromatic and wherein the ring atoms in the aromatic ring are only carbon atoms. Also included within the scope of hydrocarbyl aryl is a moiety wherein an aromatic ring comprised of only carbon ring atoms is fused to a cycloalkyl ring wherein the cycloalkyl ring in the ring system contains only carbon atoms and 5-8 carbon atoms. Also included within the term hydrocarbyl aryl groups are C1-C6 alkyl groups substituted on the aryl ring in which the alkyl groups thereon are a bridging group to another moiety. The term “hydrocarbyl aryl” excludes heteroaromatic compounds wherein at least one of the ring atoms is other than a carbon atom. The hydrocarbyl aryl group may be unsubstituted or substituted by 1, 2, or 3 substituents, which are C1-C6 alkyl, halo, or cycloalkyl. Examples of hydrocarbyl aryl compounds are phenyl, naphthyl, anthracenyl, tolyl, indanyl, xylyl, and the like and examples of

The term hydrocarbyl ar(C1-C3)alkyl refers to a hydrocarbyl aryl group, as defined herein, attached to another moiety in the molecule by an alkyl group, as defined herein, containing 1-3 carbon atoms. Examples include benzyl, phenethyl, naphthylmethyl, indanylmethyl and the like.

The terms “hydroxy” and “hydroxyl” as used herein refers to the radical —OH.

The term ‘acyl,” as used herein, refers to the (Z₃C═O) group of an organic acid having a COOH group or an organic acid derivative thereof.

The term “ketal” refers to a functional group formed by the replacement of the carbonyl group of a ketone by two alkoxy groups. Ketals are generally formed by reaction of the carbonyl group of a ketone with two alcohols having 1-6 carbon atoms, such as methanol or ethanol, or a diol having 2-6 carbon atoms, such as ethylene glycol or 2,2-dimethylpropane-1,3-diol, and the like under anhydrous conditions, in the presence of an acid.

As defined herein, an “inert solvent” is a solvent that does not react with either the reactants or products formed in the reaction. Suitable organic solvents for use in the present disclosure include, but are not limited to, alcohols having 1-6 carbon atoms, such as methanol, ethanol, isopropanol, butanol and the like; ketones having 1-6 carbon atoms, such as acetone, methyl ethyl ketone, methyl isobutyl ketone, and the like; ether solvents having 1-6 carbon atoms, such as dimethyl ether, diethyl ether, methyl ethyl ether, methyl t-butyl ether or MTBE, dipropyl ether, diisopropyl ether and the like, or cyclic ethers having 4-6 carbon atoms, such as THF, dioxane, and the like; halogenated solvents such as dichloroethane, dichloromethane, chloroform and the like; esters having 2-10 carbon atoms, such as ethyl acetate, isopropyl acetate, n-propyl acetate and the like; nitriles such as acetonitrile, propionitrile and the like; hydrocarbons having 1-10 carbon atoms, including aryl groups, such as toluene, xylene, cyclohexane, heptane, xylene and the like; dimethylsulfoxide (DMSO), N,N-dimethylformamide (DMF), N,N-dimethylacetamide (DMA) and the like; and mixtures thereof in various proportion without limitation. The use of a suitable solvent includes the use of a mixture of solvents thereof. The applicability of a particular solvent for a reaction is dependent on several factors, such as the type of reaction, the reactants, the products, and reagents used, and the like. One of ordinary skill in the art can determine the appropriate solvents for the reactions described herein.

As used herein, the term “protic” refers to a proton or a hydrogen atom or ion. The term “protic solvent,” as used herein, refers to a polar liquid compounds that have dissociable hydrogen atoms and is capable of forming a hydrogen bond with oxygen, fluorine, or nitrogen atoms.

The term “protic polar solvent,” as used herein refers to solvents which has at least one OH or NH bond and is miscible with water. Examples include water, methanol, ethanol, ammonia, and the like.

In the reactions that are described hereinbelow, functional groups may be protected by protecting groups. A protecting group, as defined herein, is a molecular framework that is introduced onto a specific functional group in a molecule containing two or more functional groups to block the reactivity of the specific functional group under reaction conditions needed to make modifications elsewhere in the molecule. To be useful, a protecting group must meet certain requirements. First, it must react selectively in good yield to protect the desired functional group and be stable under reaction conditions that are used to make modifications in the molecule elsewhere. Second, the protecting group needs to be removed selectively in good yield by readily available and preferably non-toxic reagents under conditions that do not modify other functional groups on the molecule. In an embodiment, the protecting group should form a solid derivative without generating new chiral centers and can be separated easily from the molecule after the desired modification to the molecule has been effected. Further, the protecting group should have a minimum of additional functional groups to avoid further sites of reaction during the process of modifying the molecule.

As used herein, the term “Zₓ” and “Z_{g}” refers to a Z substituent, as defined herein, that is attached to the aryl ring, such as a phenyl group, x or g times, respectively, wherein each Z substituent is the same or different and wherein x is 0, 1, 2, 3, 4, or 5 and g is 0 1, 2, 3, or 4. For example, if x is five, then the aryl, e.g., phenyl, ring has five Z substituents, wherein each Z substituent is as defined herein and may be the same or different. If x or g is four, then the aryl, e.g., phenyl, ring has four Z substituents, wherein each Z substituent is as defined herein and may be the same or different and wherein the remaining Z substituent on the aiyl, e.g., phenyl, ring is hydrogen. If x or g is three, then the aryl, e.g., phenyl, ring has three Z substituents, wherein each Z substituent is as defined herein and may be the same or different and wherein the remaining Z substituents on the aryl, e.g., phenyl, ring are hydrogen. If x or g is two, then there are two Z substituents on the aryl, e.g., phenyl, ring, which may be the same or different wherein each Z substituent on the aryl, e.g., phenyl ring, is as defined herein, and may be the same or different and wherein the remaining Z substituents on the aryl, e.g., phenyl, ring are hydrogen. If x or g is one, then there is only one Z substituent, as defined herein, on the aryl, e.g., phenyl ring, and the remaining Z substituents on the aryl, e.g., phenyl, ring are hydrogen. If x or g is zero, then there are no Z substituents on the ring, and the substituents on the aryl, e.g., phenyl, are hydrogen.

Some compounds described in the present disclosure contain the moiety

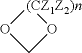

wherein Z₁ and Z₂ are as defined herein, and n is 2 or 3. This moiety represents the ketal protecting group that is formed from HO—(CZ₁Z₂)ₙ—OH. As used herein, the numbering in the ring is based on the position of atoms in the ring, regardless of whether the atom is a carbon or oxygen atom. Thus, one of the oxygen atoms in the ring is at position 2 with the carbon atom at the bottom apex being position-1 of the ring. When n is 2, the moiety becomes

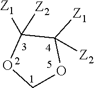

with the numbering of the atoms as shown wherein Z₁ and Z₂ are the same or different and are defined as described herein.

When n is 3, the moiety becomes

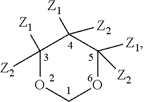

with the numbering of the atoms as shown. It is understood that each Z₁ in the above moieties may be the same or different and each Z₂ in the above moieties may be the same or different and that Z₁ and Z₂ may be the same or different. Thus, for example, when n is 3, an embodiment is

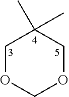

wherein the Z₁ and Z₂ substituents on carbon atoms labelled 3 and 5 are both hydrogens, while the Z₁ and Z₂ substituents on the carbon atom labelled 4 are both methyl.

As used herein, the term “acyl reducing agent” is a compound that reduces acyl groups, e.g., Z₃C═O, to a Z₃—CH₂ or Z₃—CD₂ group that are known in the art. Examples include sodium borohydride and iodine; lithium aluminum hydride and iodine; Y[N(TMS)₂]₃; lithium or sodium triethyl borohydride in the presence of silanes, such as PhSiH₃ and alkali metal base, such as, NaOH, or KOH, or NaOMe; (EtO)₃SiH; 1,1,3,3-tetramethyldisiloxane; 1,2-bis(dimethylsilyl)benzene; Tf₂O followed by reduction with sodium borohydride, Et₂Zn; Tf₂O in the presence of B(C₆F₅)₃ and TMDS, B(C₆F₅)₃ in the presence of TMDS, nickel chloride(dme) in the presence of PhSiH₃ and the like.

The term “acylating agent of formula Z₃COOXₒ”, as used herein, refers to the compound where Xₒ is H or D or acid derivative thereof, such as a halide, ester or anhydride.

The term “acyl reducing agents being deuterated” or similar term refers to an acyl reducing agent in which the hydrogen atoms therein are all replaced by deuterium. Examples include NaBD₄, LiAlD₄, and the like.

The term “acid derivative” refers to an acid halide, or ester, or anhydride of the referenced acid.

In the present disclosure, reference to a particular variable maintains the definitions given herein. Thus, for example, Z, as recited herein, refers to the definition of Z given herein.

In an embodiment, Z₁ and Z₂ are independently H or C1-C3 alkyl and Z₄ and Z₅ are independently C1-C3 alkyl, and n, Z, Z₃, Z₆, Z₇, Z₈, X₁, a, b, x and Ar are as defined herein.

In another embodiment, each pair of Z₁ and Z₂ is the same and each pair of Z₅ and Z₄ is the same. In other words, a pair of Z₁ and Z₂ are substituted on the same carbon. A pair of Z₄ and Z₅ refers to OZ₄ and OZ₅ being substituted on the same carbon. In each of these embodiments, n, Z, Z₃, Z₆, Z₇, Z₈, X₁, a, b, x and Ar are as defined herein.

In a further embodiment, Z₃ is H or unsubstituted C1-C6 alkyl, C1-C6 alkyl substituted with halogen or unsubstituted aryl, or aryl substituted with halo or C1-C6 alkyl or Z₃ is D, and n, Z, Z₁, Z₂, Z₄, Z₅, Z₆, Z₇, Z₈, X₁, a, b, x and Ar are as defined herein.

In still another embodiment, Z₃ is H or Z₃ is D, and n, Z, Z₁, Z₂, Z₄, Z₅, Z₆, Z₇, Z₈, X₁, a, b, x and Ar are as defined herein. Thus, for example, Z₃ is D when each Z₈ is D and n, Z, Z₁, Z₂, Z₄, Z₅, Z₆, Z₇, X₁, a, b, x and Ar are as defined herein.

In an embodiment, Z₆ is C1-C6 alkyl, C3-C10 cycloalkyl, hydrocarbyl aryl, or hydrocarbyl ar(C1-C3) alkyl, each of which may be unsubstituted or substituted with one or more halo or C1-C6 alkyl or C1-C6 alkoxy. In another embodiment, Z₆ is C1-C6 alkyl, C3-C6 cycloalkyl, hydrocarbyl aryl, or hydrocarbyl ar(C1-C3) alkyl, each of which may be unsubstituted or substituted with one or more halo or C1-C6 alkyl. In another embodiment, Z₆ is C1-C6 alkyl. In a still another embodiment, Z₆ is C1-C4 alkyl. Examples of Z₆ include t-butyl, phenyl, tolyl and adamantyl, and the like. In each of these embodiments, n, Z, Z₁, Z₂, Z₃, Z₄, Z₅, Z₇, Z₈, X₁, a, b, x and Ar are as defined herein.

In an embodiment, Ar is hydrocarbyl aryl or hydrocarbyl ar(C1-C3)alkyl, wherein n, Z, Z₁, Z₂, Z₃, Z₄, Z₅, Z₇, Z₈, X₁, a, b, x and Z₆ are as defined herein. In another embodiment, Ar is phenyl, wherein n, Z, Z₁, Z₂, Z₃, Z₄, Z₅, Z₇, Z₈, X₁, a, b, x and Z₆ are as defined herein.

In a still further embodiment, x is 0, 1 or 2, wherein n, Z, Z₁, Z₂, Z₃, Z₄, Z₅, Z₆, Z₇, Z₈, X₁, a, b, and Ar are as defined herein.

In another embodiment, x is 1 or 2 and each Z is independently halogen, —OZ₁₁, or C1-C10 alkyl, wherein n, Z₁, Z₂, Z₃, Z₄, Z₅, Z₆, Z₇, Z₈, X₁, a, b, and Ar are as defined herein.

In an embodiment, at least one Z is F and n, Z₁, Z₂, Z₃, Z₄, Z₅, Z₆, Z₇, Z₈, X₁, a, b, x and Ar and any additional Z, if present, are as defined herein.

In still a further embodiment, Ar is phenyl and at least one Z is F at the 4-position of Ar and n, Z₁, Z₂, Z₃, Z₄, Z₅, Z₆, Z₇, Z₈, X₁, a, b, x, and any additional Z, if present, are as defined herein.

Moreover, in an embodiment, x is 1 and Z is F, and n, Z₁, Z₂, Z₃, Z₄, Z₅, Z₆, Z₇, Z₈, X₁, a, b, and Ar and are as defined herein.

In an embodiment, Z₆ is alkyl or phenyl which is unsubstituted or substituted with C1-C6 alkyl and each Z is independently halogen (selected from F, Cl, Br, I), —OZ₁₁, C1-C10 alkyl, C2-C10 alkenyl, C4-C10 alkynyl having no terminal hydrogen atoms, CF₃, or OCF₃; wherein each Z₁₁ is independently selected from C1-C10 alkyl, C2-C10 alkenyl, C3-C10 alkynyl having no terminal hydrogen atoms, and aryl which is unsubstituted or substituted with C1-C6 alkyl, C1-C6 alkoxy, or halogen and n, Z₁, Z₂, Z₃, Z₄, Z₅, Z₇, Z₈, X₁, a, b, x and Ar are as defined herein.

In a further embodiment, Z₃ is H, D, or C1-C6 alkyl optionally substituted with one or more fluoro or C1-C3 alkoxy, and n, Z, Z₁, Z₂, Z₄, Z₅, Z₆, Z₇, Z₈, X₁, a, b, x and Ar are as defined herein.

The definitions of the various variables are applicable to the compounds and processes described herein. Moreover, all of the various permutations of the definitions of the variables, including the subsets of the definitions given herein, are contemplated by the present disclosure.

In an embodiment, the present disclosure relates to the synthesis of compounds of Formula I as defined herein:

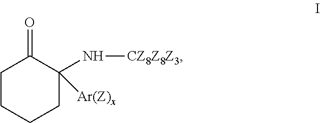

wherein Z₈, Z₃, Ar, Z and x are as defined herein.

It is prepared by a series of art recognized reactions. The starting materials for the reactions described herein are either commercially available or are prepared using well-known techniques in the art. The starting material undergoes a series of reactions to produce novel intermediates which undergo a series of reactions to produce the compound of Formula I. The synthesis for preparing a compound of Formula I can originate from any one of the novel intermediates or from the starting material. The synthesis initially described herein initially will be described going backwards without indicating the stereochemistry. Subsequently, the synthesis will be described in preparing the R isomer and the S isomer, going in the forward direction.

The last step of the synthesis of the compound of Formula I is a deketalization of a compound of Formula X or XA:

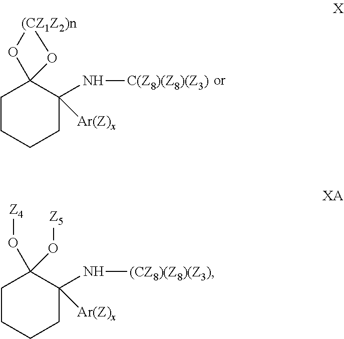

wherein Z₁, Z₂, Z₃, Z₄, Z₅, Z₈, Z, Ar, n, and x are as defined herein.

In an embodiment, the compound of Formula X or XA undergoes ketal deprotection under conditions known to one of ordinary skill in the art to form a compound of Formula 1. Deketalization, in general, is a reaction with which one of ordinary skill in the art is quite familiar. In an embodiment, deprotection is often performed by reacting the compound of Formula X or XA in acid, for example, protic acids such as HCl, HBr, HI, H₂SO₄, H₃PO₄, trifluoracetic acid (TFA), p-toluenesulfonic acid, and the like or under aprotic conditions with such reagents as Indium (ITI) trifluoromethanesulfonate in the presence of acetone, sodium tetrakis (3,5-trifluoromethylphenyl)borate, Er(OtF)₃, iodine, perchloric acid adsorbed on silica gel, bismuth nitrate, and the like under conditions effective to deprotect the ketal and form the corresponding ketone. The deprotection reaction is conducted in a polar protic solvent, for example, water, alcohol having 1 to 5 carbon atoms, such as methanol, ethanol, isopropanol, propanol, tert-butanol, t-amyl alcohol, ethylene glycol, propylene glycol, and the like or the combination of a polar protic solvent and an aprotic solvent, such as toluene, fluorobenzene, chlorobenzene, dichlorobenzene, and the like. In an embodiment, the deprotection is conducted in water. In another embodiment, the deprotection is conducted in water to which concentrated hydrochloric acid is added, i.e., it is effected with concentrated hydrochloride acid in water. The reaction is conducted at effective temperatures, such as from about room temperature to the boiling point of the solvent. In an embodiment, sufficient acid is added to form the acid salt. If the reaction produces the salt instead of the free amino compound, to convert to the free base, such as a compound of Formula I, the acid step is followed by basifying the reaction mixture of the acidization step described hereinabove with a base, such as sodium hydroxide, potassium hydroxide, potassium carbonate, sodium carbonate, sodium bicarbonate, and the like, such that the resulting pH is about 8 or greater, e.g., from about pH 8 to about pH 14, for instance at a pH of 12-13. It is conducted at effective temperatures, for example, at a temperature below 40° C., for example from about 0° C. to below 40° C. The basification reaction is conducted in a polar solvent, such as water, and the free base may be extracted into an aprotic solvent that is immiscible with water, such as ethers, for example, dimethyl ether, diethyl ether, methyl ethyl ether, methyl t-butyl ether (MTBE), di(propyl) ether, di(isopropyl) ether, or such as halogenated solvents, for example, dichlormethane (methylene chloride) or chloroform, or such as hydrocarbon solvents, for example, toluene or benzene, and the like; and mixture of solvents thereof. Bi-phasic hydrolysis, such as an aqueous base in ethyl acetate and water or aqueous acid in toluene and water can affect the formation of the free base, especially when the product is taken up in the organic layer and the reagents are in the aqueous layer. This separation facilitates the isolation of the free base from the reagents. The product is the free base of compound I.

The compound of Formula X and XA are prepared by reducing a compound of Formula IX or Formula IXA, respectively:

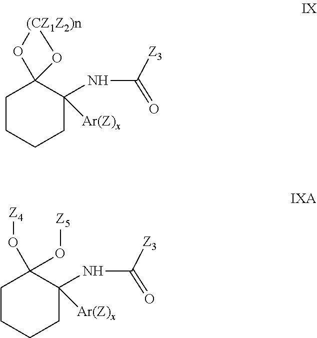

wherein Z₁, Z₂, n, Z₃, Z, x, Z₄, Z₅, and Ar are as defined herein, with an acyl reducing agent, such as lithium aluminum hydride in the presence of iodine, sodium borohydride in the presence of iodine, sodium borohydride in the presence of AlCl₃, CoCl₃/NaBH₄, BH₃ in DMS, BH₃ in THF, borane N,N-diethylaniline complex, Li(iPr)₂BH₃, BEt₃ in the presence of an alkali metal base, such as NaOH, KOH, NaOMe, and the like; Y[N(TMS)₂]₃ and HBpin; lithium or sodium triethyl borohydride in the presence of silanes, such as PhSiH₃ and alkali metal base, such as, NaOH, or KOH, or NaOMe; (EtO)₃SiH; 1,1,3,3-tetramethyldisiloxane; 1,2-bis(dimethylsilyl)benzene; Tf₂O followed by reduction with sodium borohydride; Tf₂O in the presence of B(C₆F₅)₃ and TMDS, B(C₆F₅)₃ in the presence of TMDS, nickel chloride(dme) in the presence of PhSiH₃, and the like. For preparing the compound of Formula X or XA, wherein Z₃ is D and both Z₈'s are D, the compound of Formula IX or IXA is reacted with deuterated acyl reducing agents, such as deuterated lithium aluminum hydride of the formula LiAlD₄, and in another embodiment, deuterated sodium borohydride of the formula NaBD₄, both in the presence of iodine. The acyl reducing reactions are conducted in an inert solvent, such as ethers, for example cyclic ethers of 1 to 5 carbon atoms or ethers of the formula Z₂₇—O—Z₂₈ at effective temperatures, such as wherein Z₂₇ and Z₂₈ are independently C1-C6 alkyl groups or Z₂₇ and Z₈ taken together with the oxygen atom to which they are attached form a cyclic ring of 5 or 6 carbon atoms wherein one or two of the carbon atoms may be replaced with oxygen, as long as no two oxygen atoms are adjacent to each other. Examples of ethers that may be used as the inert solvent include diethyl ether, dipropyl ether, diisopropyl ether, dibutyl ether, MTBE, tetrahydrofuran, or 1,4-dioxane; or it is conducted in other appropriate inert solvents, such as chloroform, methylene chloride, and the like; and mixture of solvents thereof. The reaction is conducted at effective temperatures, such as temperatures ranging from about −20° C. to the boiling point of the solvent.

The compound of Formula IX or IXA is prepared by reacting the amine of formula VIII or VIIIA,

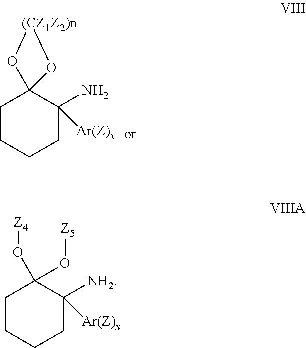

respectively, wherein Z₁, Z₂, n, Ar, Z, x, Z₄, and Z₅ are as defined herein, with an acylating agent Z₃COOXₒ, wherein Z₃ is as defined herein and Xₒ is H or D, or with an acid derivative thereof, such as the corresponding acid halide, such as an acid chloride, corresponding ester, for example, Z₃COOZ₁₂, or acid anhydride, such as Z₃C(═O)—O—C(═O)Z₁₂, wherein for example, Z₁₂ is C1-C6 alkyl or aryl which alkyl groups and aryl groups are unsubstituted or are substituted with halogen, or C1-C6 alkoxy, or arylC₁-C6alkyl, C1-C6 alkyl and the like, under amide forming conditions.

For example, the amide formation can be effected by reacting the amine of Formula VIII or VIIIA with Z₃C(O)Cl in the presence of an aqueous base, such as NaOH or KOH under Schotten Baumann Reaction conditions. In addition, other acylating agents or acyl transfer agents known in the art may be used.

When Z₃ is D and both Z₈'s are D, then the amine of Formula VIII or VIIIA, respectively is reacted with DCO₂D under amide forming conditions.

If the amide is formed from a carboxylic acid, then the reaction may be conducted in the presence of coupling agents known in the art, such as carbodiimide, under amide formation effective methods, with, for example, DCC, DIC, CMC, EDC, and the like or with other carboxylic acid activators, such as HATU, HBTU, BOP, PyBOP, DEPBT, CDI, TCDI, (2-thiophen-2-ylmethyl)phenyl)boronic acid, 5-methoxy-2-iodophenylboronic acid, ammonia-borane, tetramethyl orthosilicate, triphenylphosphine, XtalFluor-E, N-formylpyrrolidine with trichlorotriazine, DIPEA, DABCO, or any other catalyst and dehydrating agent known in the art, and the like.

The reaction is conducted in the presence of an inert solvent known to one of ordinary skill in the art, such as DMF, N-methylpyrrolidone (NMP), N,N-dimethylacetamide (DMA), DMSO, acetonitrile, ethyl acetate, isopropyl acetate, methylene chloride or chloroform or ethers containing 1-6 carbon atoms, such as THF or dioxane, diethyl ether, dimethyl ether, t-butyl methyl ether; hydrocarbon solvents like heptane and toluene, and the like; and mixture of solvents thereof. The reaction is conducted at sufficient temperatures to form the amide. In an embodiment, the reaction is conducted at a temperature ranging from about 0° C. to the boiling point of the solvent.

In another embodiment, the acid Z₃COOXₒ is reacted with the compound of Formula VIII or VIIIA in the presence of PPh₃ and NBS and Et₃N to form a compound of Formula IX or IXA, respectively. In an embodiment, the reaction is conducted in an inert aprotic polar solvent, such as acetonitrile, ethyl acetate, isopropyl acetate, methylene chloride or chloroform or ethers containing 1-6 carbon atoms, such as THF or dioxane, diethyl ether, dimethyl ether, t-butyl methyl ether; hydrocarbon solvents like heptane and toluene, and the like; and mixture of solvents thereof at temperatures effective to from the amide of Formula IX or IXA, such as from about 0° C. to room temperature.

The compound of Formula VIII or VIIIA is formed from acid hydrolysis of a sulfinamide of Formula VII or VIIA under amine forming conditions.

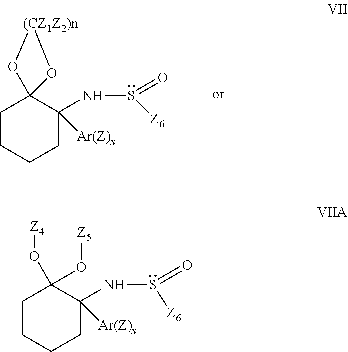

wherein Z₁, Z₂, n, Ar, Z₆, Z, x, Z₄, and Z₅ are as defined herein.

For example, the amine of Formula VIII or VIIIA is formed by acid hydrolysis of the sulfinamide of Formula VII or VIIA, respectively, for example, using a strong protic acid such as HX₁ or nitric acid or sulfuric acid, phosphoric acid or trifluoracetic acid, or para-toluenesulfonic acid wherein X₁ is a halide. The reaction is conducted in a polar protic solvent, such as those listed hereinabove at temperatures sufficient to effect the hydrolysis of the sulfinamide to an amine. For example, the reaction may be conducted at temperatures ranging from about 0° C. to the boiling point of the solvent.

The sulfinamide of Formula VII or VIIA is formed by reacting an aryl nucleophilic agent Nu, capable of delivering an aryl nucleophilic moiety of the formula Ar(Z)ₓ, as defined herein, with an imine of Formula V or VA, respectively under arylation reaction conditions:

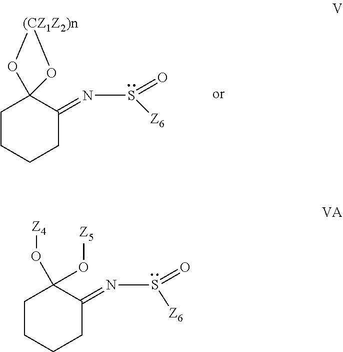

wherein Z₁, Z₂, n, Z₆, Z₄, and Z₅ and x are as defined herein. Examples include molecules in which the moiety Ar(Z)ₓ is present with an organometallic, such as a organomagnesium compound, such as a Grignard reagent MgBrAr(Z)ₓ, or organolithium compounds, e.g., LiAr(Z)ₓ, or organo-potassium, sodium, or aluminum, such as organoaluminates, or other organonmetallic, such as organocopper, zinc, tin, and the like, an organoborate, wherein the moiety Ar(Z)ₓ is bonded to a boron atom or arylphosphonium salt such as a tetraaiylphosphonium salt having the moiety [Ar(Z)ₓ]₄ P⁺, and the like. Examples when the moiety Ar(Z)ₓ is present with an organometallic include molecules of the formula Ar(Z)ₓMgX, Ar(Z)ₓLi, Ar(Z)xZnX2, InAr(Z)xX where Ar, Z and x are as defined hereinabove and X is a halide, such as Br or Cl. When an organolithium is utilized, the use of additives, such as BF₃·OEt₂, tetramethylethylenediamine (TMEDA), enhances the yield. Since the aryl nucleophilic agents react with air and water, the reaction is conducted in an inert atmosphere, such as under nitrogen or inert noble gas. The reaction is conducted under conditions to effect the coupling of Ar(Z)ₓ with the six-membered ring. The reaction is generally effected in an inert solvent, such as ethers, such as dimethyl ether, diethyl ether, methyl ethyl ether, methyl tributyl ether, dipropyl ether, diisopropyl ether, THF, or 1,4-dioxane, or such as hydrocarbon solvents, such as toluene, benzene, hexanes, or n-heptane, and the like, and mixture of solvents thereof, under conditions effective to form the sulfinamide of Formula VII or VIIA respectively. The reaction can be conducted at an effective temperature for coupling, such as a temperature ranging from about −78° C. to about room temperature when conducted in the aforementioned organic solvents listed hereinabove.

Other methods for forming sulfinamides are described in an article by Elzbieta Wojaczynska and Jacek Wojaczynski, entitled “Modern Stereoselective Synthesis of Chiral Sulfinyl Compounds,” Chem. Rev. 2020, 120, 4578-4611, the contents of which are incorporated by reference.

To one skilled in the arts, other recognized methods to making structures such as VIII and VIIIA include the asymmetric addition of alkylmetals to chiral imines such as one containing alpha-naphthylethyl group as a chiral auxiliary.

The nucleophilic agents, Nu, are prepared by art by art-recognized techniques. For example, Grignard reagents are prepared by treating an aryl halide, such as bromide or chloride, with the organometal metal, such as magnesium metal, in the presence of an inert solvent, such as THF, in the absence of water and air. Organolithium compounds are also prepared by art recognized reaction. For example, aryl lithium compounds are formed by reacting aryl halides with lithium metal.

The compound of Formula V or VA is prepared by reacting spiro compound of Formula III or IIIA, respectively:

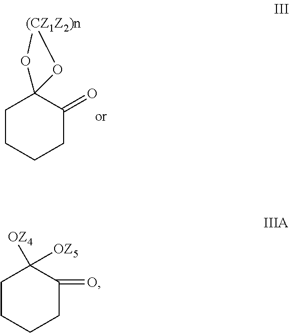

wherein Z₁, Z₂, Z₄, Z₅, and n are as defined herein with a sulfinyl compound

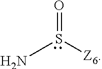

wherein Z₆ is as defined herein in the presence of a titanium compound of the formula Ti(OZ₇)ₐX_{1b}, wherein Z₇, a, X₁ and b are as defined herein or other catalyst such as CuSO₄. Examples of titanium compounds include Ti(OEt)₄, Ti((iOPr)₄, TiCl(OiPr)₃, TiCl₂(OiPr)₂, and TiCl₃(OiPr). These titanium compounds are either commercially available or can be prepared by the skilled artisan. The reaction is conducted in an inert solvent, such as methylene chloride or chloroform or ethers, such as THE or dioxane, diethyl ether, dimethyl ether, t-butylmethyl ester; or hydrocarbon solvents like toluene, benzene, ethylbenzene, pentane, hexane, cyclohexane, petroleum ether, tetrahydrofuran, and the like; and mixture of solvents thereof. The reaction is conducted at effective temperatures. In an embodiment, the reaction is conducted at about room temperature to the boiling point of the solvent.

The compounds of Formula III and Formula IIIA are prepared by art-recognized procedures. Polyhydric alcohols, or polyols, having 1,2 and 1,3 hydroxy conformations can react with a ketone to form a cyclic ketal. For example, with respect to preparing compounds of Formula III, 1,2-cyclohexanedione is reacted with a diol of the formula HO—(CZ₁Z₂)ₙ—OH such as ethylene glycol, propylene glycol, 2,2-dimethylpropane-1,3-diol in the presence of an acid, such as HCl, HNO₃, H₂SO₄, phosphoric acid, methane sulphonic acid, p-toluenesulfonic acid, and the like, wherein n and each Z₁ and Z₂ are as defined hereinabove. The reaction is conducted in an inert solvent, such as such as methylene chloride or chloroform or ethers, such as THE or dioxane, diethyl ether, dimethyl ether, t-butylmethyl ester; or hydrocarbon solvents like toluene, benzene, ethylbenzene, pentane, hexane, cyclohexane, petroleum ether, tetrahydrofuran, and the like; and mixture of solvents thereof. The reaction is conducted at effective temperatures, such as ranging from about room temperature to the boiling point of the solvent. Water may be removed from the mixture during the reaction, such as physically with a Dean-Stark trap or similar apparatus, or chemically with a desiccant such as molecular sieves or drying salts in order to drive the equilibrium toward the ketal product.

Compounds of Formula IIIA are prepared similarly. Instead of using a diol to react with 1,2-cyclohexanediones, alcohols of Z₅OH and Z₄OH are utilized wherein Z₅ and Z₄ are as defined hereinabove. In an embodiment Z₈ and Z₄ are the same. The reaction is conducted in the presence of an acid catalyst, most typically, homogeneous catalysis is employed using a protic acid (Brønsted-Lowry acid). For example, sulfuric acid, hydrochloric acid, phosphoric acid, p-toluenesulfonic acid, methane sulfonic acids and mixtures of these are known to catalyze ketal formation. Lewis acids, e.g., aprotic acids, have also been used to catalyze ketal and acetal formation from alcohols. For example, Clerici et al., Tetrahedron 54, 15679-90 (1998) employ titanium tetrachloride in the presence of an ammonia or amine to affect the reaction of methanol with various aldehydes in the presence of ammonia or amine.

The reaction is conducted in an inert solvent, such as cyclohexane, hexane, pentane, petroleum ether, benzene, toluene, ethylbenzene, and the like. The reaction is conducted at effective temperatures, such as ranging from about room temperature to the boiling point of the solvent. Water may be removed from the mixture during the reaction, such as with a Dean-Stark trap or similar apparatus, in order to drive the equilibrium toward the ketal product.

Acid addition salts, such as the pharmaceutically acceptable salts of the compounds of Formula I are prepared therefrom with pharmaceutically acceptable acids by standard techniques to form the pharmaceutically acceptable salts thereof. For example, to convert to the halide salt, the compound of Formula I is acidified with HX₃ where X₃ is Cl or Br, with stirring at a temperature ranging from room temperature to the boiling point of the polar solvent, The reaction may be conducted in a polar protic solvent such as water, an alcohol having 1-4 carbon atoms, such as methanol, ethanol, propanol, isopropanol, or tert-butanol, or a polar solvent, such as ether, for example, diethyl ether, methyl ethyl ether, methyl tributyl ether, or dipropyl ether or cyclic ether, such as THE or dioxane, a halogenated solvent, such as dichloromethane (methylene chloride) or chloroform, or a hydrocarbon solvent, such as toluene or benzene, and the like, to produce a pharmaceutically acceptable salt of a compound of Formula I.

Thus, summarizing the above, starting from 1,2-cyclohexanedione, the compound of Formula I is prepared from the cyclic ketal, as depicted in Scheme 1 or the non-cyclic ketal, as depicted in Scheme II:

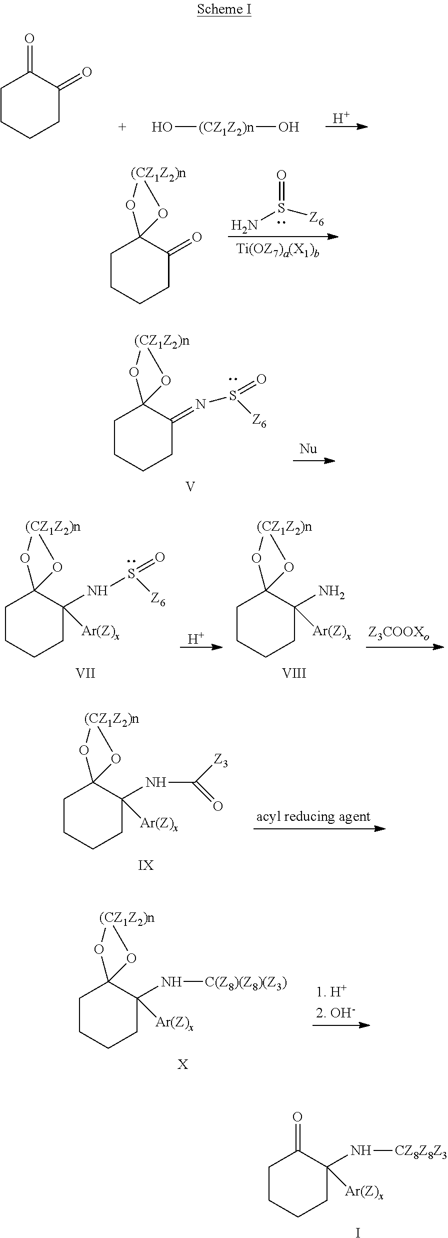

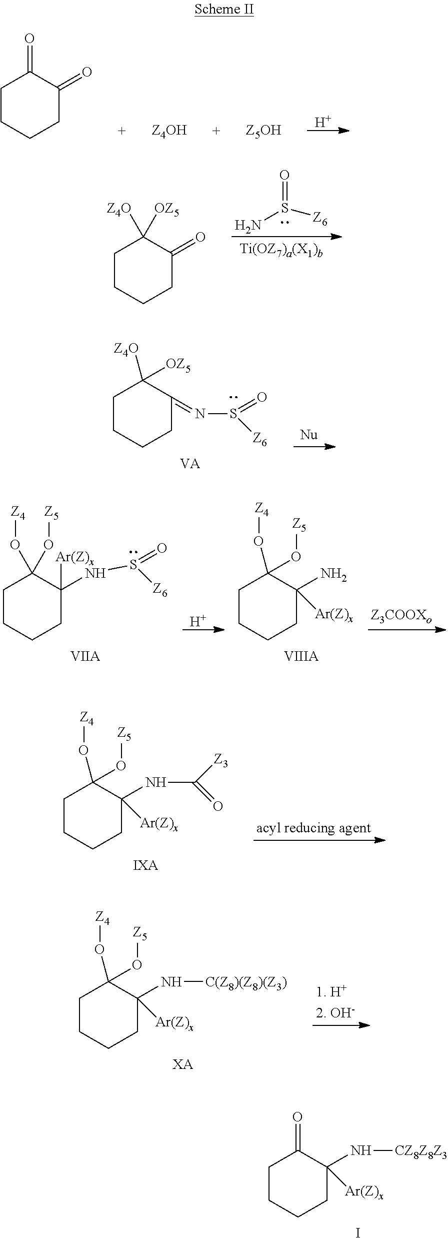

Using the above procedure, the R and S isomers of Formula I can be prepared, the contents of which are incorporated by reference. For example, utilizing

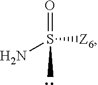

which is the R isomer of the sulfinamide used hereinabove, the R isomer of Formula 1, designated as IR

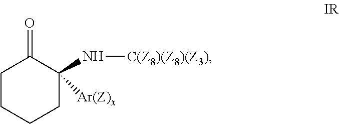

is prepared, where Z₈, Z₃, Z, Ar, Z, Z₆ and x are as defined herein. The procedure described hereinabove is incorporated by reference.

The R isomer of the sulfinamide, designated as compound of Formula IVR

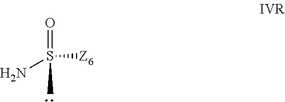

wherein Z₆ is as defined hereinabove is reacted with compounds of Formula III

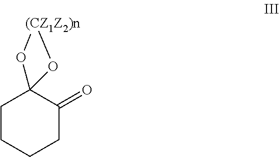

or Formula IIIA

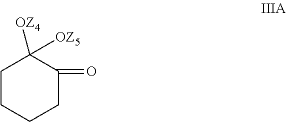

wherein Z₁, Z₂, n, Z₄, and Z₅ are as defined hereinabove to form compounds of Formula VR and VAR respectively,

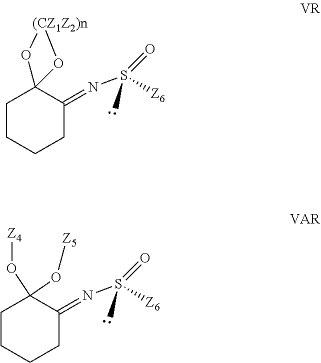

wherein i, Z₂, Z₄, Z₅, Z₆, an n are as defined herein in the presence of a titanium compound of the formula Ti(OZ₇)ₐX_{1b}, or CuSO₄, wherein Z₇, a, X₁ and b are as defined herein. Examples of titanium compounds include Ti(OEt)₄, Ti(iOPr)₄, TiCl(OiPr)₃, TiCl₂(OiPr)₂, and TiCl₃(OiPr). These titanium compounds are either commercially available or can be prepared by the skilled artisan.

The reaction is conducted in an inert solvent, such as ether solvents, such as dimethyl ether, diethyl ether, methyl ethyl ether, methyl t-butyl ether (MTBE), dipropyl ether, diisopropyl ether, and the like; halogenated solvents such as dichloroethane, dichloromethane, chloroform and the like; hydrocarbon solvents like toluene, benzene, ethylbenzene, pentane, hexane, cyclohexane, petroleum ether, and the like; and mixture of solvents thereof. The reaction is conducted at effective temperatures. In an embodiment, the reaction is conducted at about room temperature to the boiling point of the solvent.

The imines of Formula V and VAR are reacted with an aryl nucleophilic agent Nu, capable of delivering an aryl nucleophilic moiety of the formula Ar(Z)ₓ, as defined herein, under arylation reaction conditions and form compounds of Formula VIIR and VIIAR, respectively:

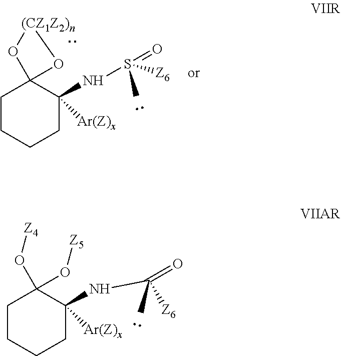

wherein Z₁, Z₂, n, Z₆, Z₄, Z₅, Ar, Z and x are as defined herein. Examples include molecules in which the moiety Ar(Z)ₓ is present in an organometallic, such as an organomagnesium compound, such as a Grignard reagent MgBrAr(Z)ₓ, or organolithium compounds, e.g., LiAr(Z)ₓ, or organo-potassium, sodium, or aluminum, such as organoaluminates, organocopper, zinc, tin, and the like; or an organoborate, wherein the moiety Ar(Z)ₓ is bonded to a boron atom or arylphosphonium salt such as a tetraaiylphosphonium salt having the moiety [Ar(Z)ₓ]₄ P⁺. For instance, organometallics examples include molecules of the formula Ar(Z)ₓMgX, Ar(Z)ₓLi, Ar(Z)xZn(X)₂. InAr(Z)xX, where Ar, Z and x are as defined hereinabove and X is a halide, such as Br or Cl. When an organolithium is utilized, the use of additives, such as BF₃·OEt₂, tetramethylethylenediamine (TMEDA), and the like, enhances the yield. Since the aryl nucleophilic agents react with air and water, the reaction is conducted in an inert atmosphere, such as under nitrogen or inert noble gas. The reaction is conducted under conditions to effect the coupling of Ar(Z)ₓ with the six-membered ring. The reaction is generally effected in an inert solvent, such as ethers having 1-6 carbon atoms, such as dimethyl ether, diethyl ether, methyl ethyl ether, methyl tributyl ether, dipropyl ether, diisopropyl ether, THF, or 1,4-dioxane; or such as hydrocarbon solvents, such as toluene, benzene, hexanes, or n-heptane, and the like; or mixture of solvents thereof, under conditions effective to form the sulfinamide of Formula VII or VIIA respectively. The reaction can be conducted at an effective temperature for coupling, such as a temperature ranging from about −78° C. to about room temperature when conducted in the aforementioned organic solvents listed hereinabove.

The nucleophilic agents, Nu, are prepared by art by art-recognized techniques. For example, Grignard reagents are prepared by treating an aryl halide, such as aryl bromide or aryl chloride, with an organometal, such as magnesium metal, in the presence of an inert solvent, such as THF, in the absence of water and air. Organolithium compounds are also prepared by art recognized reaction. For example, aryl lithium compounds are formed by reacting aiyl halides with lithium metal.

Acid hydrolysis of the compounds of Formula VIIR or VIIAR produces the amine of VIIIR and VIIIAR, respectively,

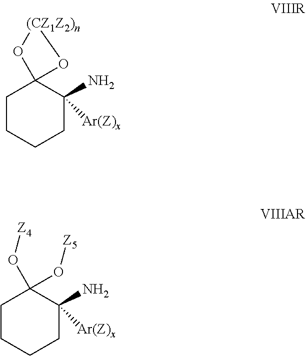

wherein Z₁, Z₂, n, Ar, Z, x, Z₄, and Z₅ are as defined herein.

For example, the amine of Formula VIIIR or VIIIAR is formed by acid hydrolysis of the sulfinamide of Formula VIIR or VIIAR, respectively, using a strong acid such as HX₁ or nitric acid or sulfuric acid, phosphoric acid or trifluoracetic acid, or para-toluenesulfonic acid wherein X₁ is a halide. The reaction is conducted in a polar protic solvent, such as those listed hereinabove at temperatures sufficient to effect the hydrolysis of the sulfinamide to an amine. For example, the reaction may be conducted at temperatures ranging from about 0° C. to the boiling point of the solvent.

The amines of Formula VIII R and VIIIAR are converted to the corresponding amides of Formula IXR and IXAR, respectively under amide reaction conditions:

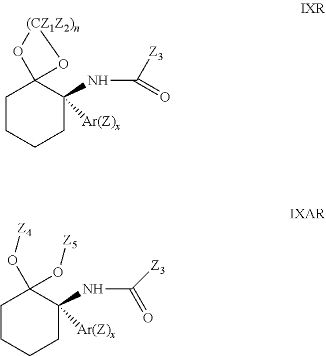

where Z₁, Z₂, n, Z₃, Z₄, Z₅, Ar, Z and x are as defined herein.

The compound of Formula IX or IXA is prepared by reacting the amine of Formula VIII or VIIIA, respectively wherein Z₁, Z₂, n, Ar, Z, x, Z₄, and Z₅ are as defined herein, with an acylating agent, Z₃COOXₒ, wherein Z₃ is as defined herein and Xₒ is H or D, or with an acid derivative thereof, such as the corresponding acid halide, such as an acid chloride, corresponding ester, for example, Z₃COOZ₁₂, or acid anhydride, such as Z₃C(═O)—O—C(═O)Z₁₂, wherein for example, Z₁₂ is C1-C6 alkyl or aryl which alkyl groups and aryl groups are unsubstituted or are substituted with halogen, or C1-C6 alkoxy, or arylC₁-C6alkyl, or C1-C6 alkyl under amide forming conditions. For example, the amide formation can be effected by reacting the amine of Formula VIII or VIIIA with Z₃C(O)Cl in the presence of an aqueous base, such as NaOH or KOH under Schotten Baumann Reaction conditions In addition, other acylating agents or acyl transfer agents known in the art may be used.

When Z₃ is D and both Z₈'s are both D, then the amine of Formula VIII or VIIIA, respectively is reacted with DCO₂D under amide forming conditions.

If the amide is formed from a carboxylic acid, then the reaction is conducted in the presence of coupling agents known in the art, such as carbodiimides under amide formation conditions, with, for example, DCC, DIC, CMC, EDC, and the like or with other carboxylic acid activators, such as HATU, HBTU, BOP, PyBOP, DEPBT, CDI, TCDI, (2-thiophen-2-ylmethyl)phenyl)boronic acid, 5-methoxy-2-iodophenylboronic acid, ammonia-borane, tetramethyl orthosilicate, triphenylphosphine, XtalFluor-E, N-formylpyrrolidine with trichlorotriazine, DIPEA, DABCO, or any other catalyst and dehydrating agent known in the art, and the like.

The reaction is conducted in the presence of an inert solvent known to one of ordinary skill in the art, such as DMF, N-methylpyrrolidone (NMP), N,N-dimethylacetamide (DMA), DMSO, acetonitrile, ethyl acetate, isopropyl acetate, methylene chloride or chloroform or ethers containing 1-6 carbon atoms, such as THF or dioxane, diethyl ether, dimethyl ether, t-butyl methyl ether; hydrocarbon solvents like heptane and toluene, and the like; and mixture of solvents thereof. The reaction is conducted at sufficient temperatures to form the amide. In an embodiment, the reaction is conducted at a temperature ranging from about 0° C. to the boiling point of the solvent.

In another embodiment, the acid Z₃COOXₒ is reacted with the compound of Formula VIIIR or VIIIAR in the presence of PPh₃ and NBS and Et₃N to form a compound of Formula IXR or IXAR, respectively. In an embodiment, the reaction is conducted in an inert aprotic polar solvent, such as acetonitrile, ethyl acetate, isopropyl acetate, methylene chloride or chloroform or ethers containing 1-6 carbon atoms, such as THF or dioxane, diethyl ether, dimethyl ether, t-butylmethyl ester; hydrocarbon solvents like heptane and toluene, and the like; and mixture of solvents thereof at temperatures effective to from the amide of Formula IXR or IXAR, such as from about 0° C. to room temperature.

The amide of Formula IXR and IXAR are reduced by acyl reducing agents known in the art to produce the compounds of XR and XAR:

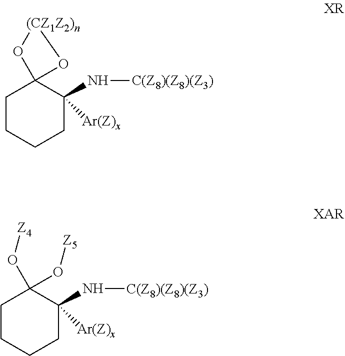

wherein Z₁, Z₂, n, Z₅, Z₃, Ar, Z, Z₄, Z₅, and x are as defined herein. Examples of acyl reducing agents, include lithium aluminum hydride in the presence of iodine, sodium borohydride in the presence of iodine, sodium borohydride in the presence of AiCl₃, CoCl₃/NaBH₄, BH₃ in DMS, BH₃ in THF, borane N,N-diethylaniline complex, Li(iPr)₂BH₃. BEt₃ in the presence of an alkali metal base, such as NaOH, KOH, NaOMe, and the like; Y[N(TMS)₂]₃ and HBpin; lithium or sodium triethyl borohydride in the presence of silanes, such as PhSiH₃ and alkali metal base, such as, NaOH, or KOH, or NaOMe; (EtO)₃SiH; 1,1,3,3-tetramethyldisiloxane; 1,2-bis(dimethylsilyl)benzene; Tf₂O followed by reduction with sodium borohydride; Tf₂O in the presence of B(C₆F₅)₃ and TMDS; B(C₆F₅)₃ in the presence of TMDS; nickel chloride(dme) in the presence of PhSiH₃; and the like. For preparing the compound of Formula X or XA, wherein Z₃ is D and both Z₈'s are D, the compound of Formula IX or IXA is reacted with deuterated acyl reducing agents, such as deuterated lithium aluminum hydride of the formula LiAlD₄, and in another embodiment, deuterated sodium borohydride of the formula NaBD₄, both in the presence of iodine. The acyl reducing reactions are conducted in an inert solvent, such as ethers, for example cyclic ethers of 1 to 6 carbon atoms and one or two oxygen atoms or ethers of the formula Z₂₇—O—Z₂₈ at effective temperatures, wherein Z₂₇ and Z₂₈ are independently C1-C6 alkyl group or Z₂₇ and Z₂₈ taken together with the oxygen atom to which they are attached form a cyclic ring and wherein one of the carbon atoms in the ring may be replaced with an oxygen atom, as long as no two oxygen atoms are adjacent to each other. Examples of ethers that may be used include diethyl ether, dipropyl ether, diisopropyl ether, dibutyl ether, MTBE, tetrahydrofuran, or 1,4-dioxane, and the like. The reaction may also be conducted in other appropriate inert solvents, such as chloroform, methylene chloride, and the like; and mixture of solvents thereof. The reaction is conducted at effective temperatures, such as temperatures ranging from about −20° C. to the boiling point of the solvent.

The next step of the synthesis of the compound of Formula IR is a deketalization of a compound of Formula XR or XAR.

In an embodiment, the compound of Formula XR or XAR undergoes ketal deprotection under conditions known to one of ordinary skill in the art to form a compound of Formula IR. One of ordinary skill in the art is quite familiar with deketalization reactions of this sort. In an embodiment, deprotection is often performed by reacting the compound of Formula XR or XAR in acid, for example, protic acids such as HCl, HBr, HI, H₂SO₄, H₃PO₄, trifluoracetic acid (TFA), p-toluenesulfonic acid, and the like, or under aprotic conditions with such reagents as Indium (III) trifluoromethanesulfonate in the presence of acetone, sodium tetrakis (3,5-trifluoromethylphenyl)borate, Er(OtF)₃, iodine, perchloric acid adsorbed on silica gel, bismuth nitrate, and the like under conditions effective to deprotect the ketal and form the corresponding ketone. The deprotection reaction is conducted in a polar protic solvent, for example, water, alcohol having 1 to 5 carbon atoms, such as methanol, ethanol, isopropanol, propanol, tert-butanol, t-amyl alcohol, ethylene glycol, propylene glycol, and the like or the combination of a polar protic solvent and an aprotic solvent, such as toluene, fluorobenzene, chlorobenzene, dichlorobenzene, and the like. In an embodiment, the deprotection is conducted in water. In another embodiment, it is effected in water to which concentrated hydrochloric acid is added, i.e., it is effected in concentrated hydrochloric acid in water. The reaction is conducted at effective temperatures, such as from about room temperature to the boiling point of the solvent. In an embodiment, sufficient acid is added to form the acid salt. If the salt is formed, to convert to the free base, such as a compound of Formula I, the acid step is followed by basifying the reaction mixture of the acidization step described hereinabove with a base, such as sodium hydroxide, potassium hydroxide, potassium carbonate, sodium carbonate, sodium bicarbonate, and the like, such that the resulting pH is about 8 or greater, e.g., from about pH 8 to about pH 14, for instance at a pH of 12-13. It is conducted at effective temperatures, for example, at a temperature below 40° C., for example from about 0° C. to below 40° C. The basification reaction is conducted in a polar solvent, such as water, and the free base may be extracted into an aprotic solvent that is immiscible with water, such as ethers, for example, dimethyl ether, diethyl ether, methyl ethyl ether, methyl tributyl ether, dipropyl ether, diisopropyl ether, or MTBE; or such as halogenated solvents, such as dichlormethane (methylene chloride) or chloroform, or such as hydrocarbon solvents, such as toluene or benzene, and the like. Bi-phasic hydrolysis, such as an aqueous base in ethyl acetate and water or aqueous acid in toluene and water can affect the preparation of the free amine, especially when the product, i.e., the free amine is taken up in the organic layer and the reagents are in the aqueous layer. This separation facilitates the isolation of the free base from the reagents. The product is the free base of compound IR.

Acid addition salts, such as the pharmaceutically acceptable salts of the compounds of Formula IR are prepared therefrom with pharmaceutically acceptable acids by standard techniques. For example, to convert to the halide salt, the compound of Formula IR is acidified with HX₃ where X₃ is Cl or Br, with stirring at a temperature ranging from room temperature to the boiling point of the solvent. The reaction may be conducted in a polar protic solvent, such as water, an alcohol having 1-4 carbon atoms, such as methanol, ethanol, propanol, isopropanol, butanol, iso-butanol, or tert-butanol, or a polar solvent, such as ether, for example, diethyl ether, methyl ethyl ether, methyl tributyl ether, or dipropyl ether, a halogenated solvent, such as dichloromethane (methylene chloride) or chloroform, or a hydrocarbon solvent, such as toluene or benzene, and the like, to produce a pharmaceutically acceptable salt of a compound of Formula I.

Thus, summarizing the above, starting from 1,2-cyclohexanedione, the compound of Formula IR is prepared from the cyclic ketal, as depicted in Scheme III or the non-cyclic ketal, as depicted in Scheme IV:

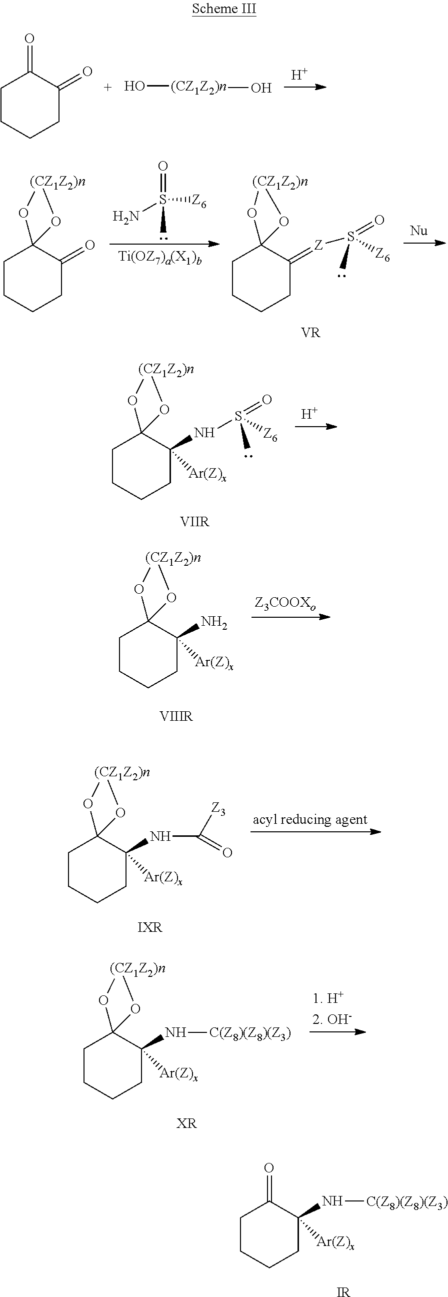

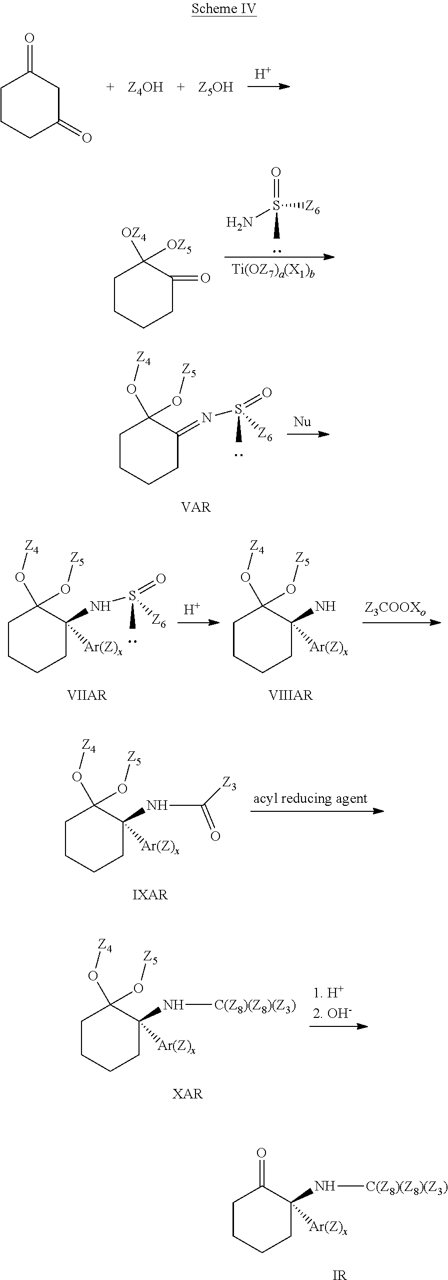

Using the teachings hereinabove, and utilizing the S isomer of the sulfinamide of the formula

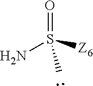

the S isomer of Formula I, designated as IS, can be prepared,

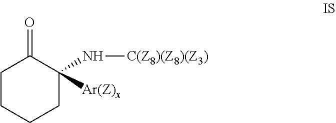

where Z₈, Z₃, Ar, Z, Z₆ and x are as defined herein. The procedures and their descriptions described hereinabove are incorporated by reference, with the understanding that the corresponding S isomer of the sulfinamide is substituting for the R isomer of the sulfinamide, whenever it appears in the description. Accordingly, the following schemes V (cyclic ketal) and VI (non-cyclic ketal) can be used to prepare the S isomer of Formula IS.

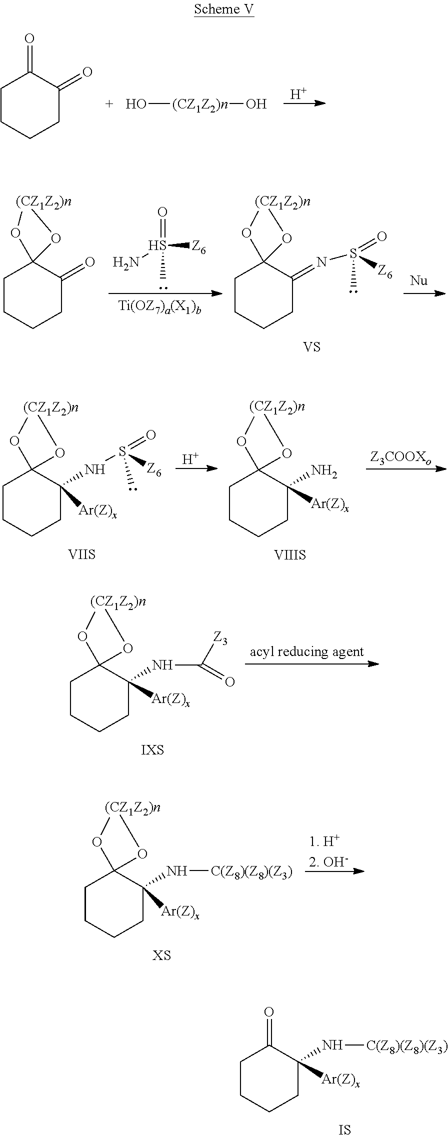

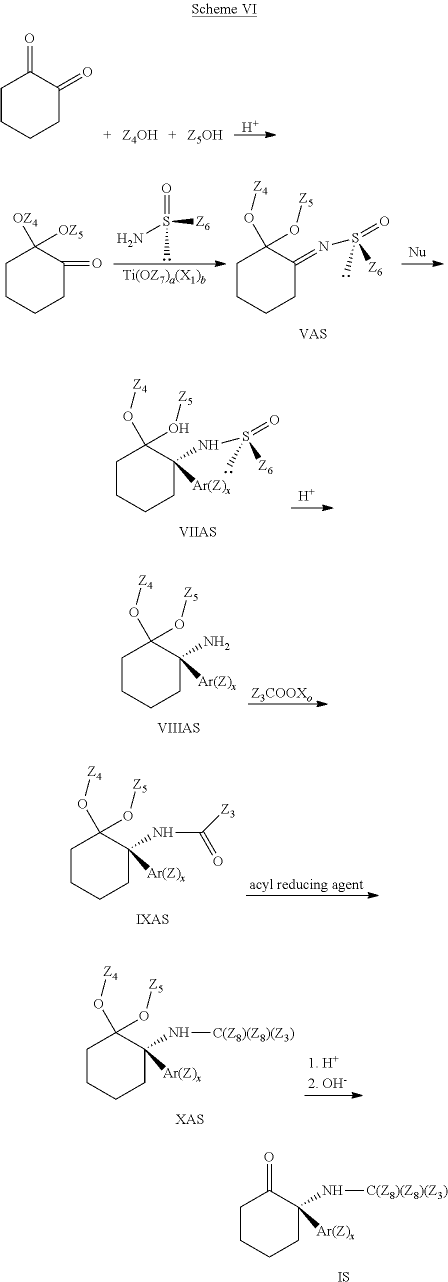

Another procedure for preparing the compound of Formula I is a variation of the schemes after the amine for compound for Formula VIII is formed. Illustrating with the compound of Formula VIII, in this variation, the amine

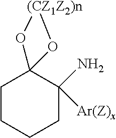

is subjected to reductive amination with Z₃C(═O)Z₈ to form the corresponding amine of the formula

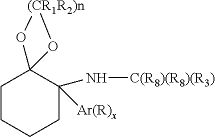

in the presence of reductive amination agents known in the art. Examples include sodium cyanoborohydride (NaBH₃CN), sodium borohydride sodium tri-acetoxyborohydride (NaBH(OAc)₃, and the like in the reductive amination solvent, such as DCE and the like.

The corresponding enantiomers can be isolated from the racemic compound by techniques known in the art. Examples include, but are not limited to, the formation of chiral salts and the use of chiral or high performance liquid chromatography “HPLC” and by the formation and crystallization of chiral salts. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

Alternatively, chemoselective reductive amination can be effected from compounds of Formula VIIIR and VIIIAR and VIIIS and VIIIAS with Z₃C(═O)Z₈ to form the corresponding amine of Formula XR, XAR, XS and XAS using chemoselective reductive amination agents known to one of ordinary skill in the art, such as [RuCl2(p-cumene)]₂/Ph₂SiH₂, dibutyltin chloride in the presence of phenylsilane and the like. The reactions are conducted in inert solvents, such as ethers, cyclic ethers, DCM, and the like.

In an embodiment, the disclosure describes a method for preparing the compound of the formula:

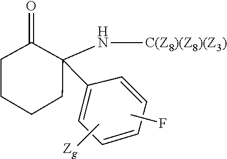

or pharmaceutically acceptable salts thereof
which method comprises

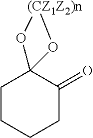

(a) reacting with t-butylsulfinamide in an inert solvent in the presence of Ti(OEt)₄ to form an imine of Formula V

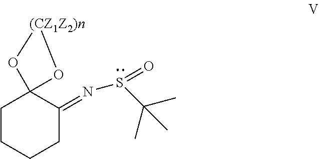

(b) reacting the imine of Formula V with a Grignard reagent of the formula:

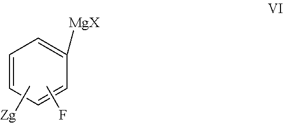

under Grignard forming conditions to form a sulfinamide product of Formula VII, wherein X is halo:

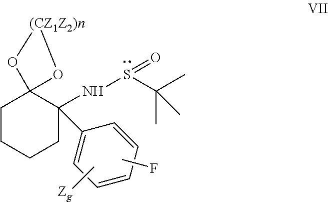

(c) reacting the compound of Formula VII with acid to form the corresponding amine of Formula VIII:

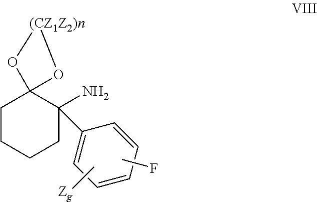

(d) reacting the amine of Formula VIII with an acylating agent of the formula Z₃COOXₒ wherein Xₒ is H or acid derivatives thereof or D under amide forming conditions to form the amide of Formula IX:

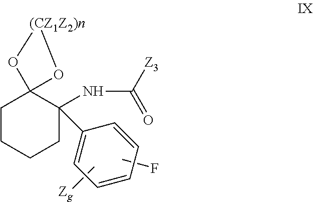

(e) reducing the acyl group (C═O) in formula IX with an acyl reducing agent selected from sodium borohydride or deuterated sodium borohydride, both in the presence of iodine, to form the compound of Formula X:

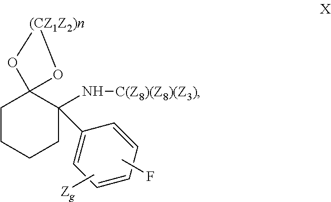

and
   (f) deprotecting the ketal of step (e) to form the compound of Formula I wherein
   each Z₁ is independently H or C1-C6 alkyl;
   each Z₂ is independently H or C1-C6 alkyl;
   n is 2 or 3;
   each Z₈ is independently H or D;
   Z₃ is hydrogen, alkyl optionally substituted with one or more halogen or C1-C6 alkoxy or aryl optionally substituted with one or more C1-C6 alkyl, C1-C6 alkoxy or halogen or Z₃ is D;
   each Z is independently halogen (selected from F, Cl, Br, I), —OZ₁₁, C1-C10 alkyl, C2-C10 alkenyl, C4-C10 alkynyl having no terminal hydrogen atoms, CF₃, or OCF₃; wherein each Z₁ is C1-C10 alkyl, C2-C10 alkenyl, C3-C10 alkynyl having no terminal hydrogen atoms, or aryl, and
   g is 0, 1, 2, 3, or 4.

In the above reaction scheme, the deuterated compounds are formed when both Z₃ is D and deuterated sodium borohydride in the presence of iodine is used as the acyl reducing agent. On the other hand, if a non-deuterated acyl reducing agent is used and a non-deuterated acylating is utilized, the non-deuterated product of Formula I is produced.

In an embodiment, Z₃ is hydrogen or alkyl having 1-6 carbon atoms or D. In another embodiment, each Z is independently halogen (selected from F, Cl, Br, I), —OZ₁₁, C1-C10 alkyl, C2-C10 alkenyl, C4-C10 alkynyl having no terminal hydrogen atoms, CF₃, or OCF₃; wherein each Z₁ is C1-C10 alkyl, C2-C10 alkenyl, C4-C10 alkynyl having no terminal hydrogen atoms, or aryl.

The compounds of Formula I can be prepared from the non-cyclic ketals similarly starting from the compound of the formula:

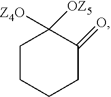

wherein the intermediates in the scheme are the corresponding non-cyclic ketals.

In another embodiment, the R isomer of the following formula can be prepared using the techniques described hereinabove:

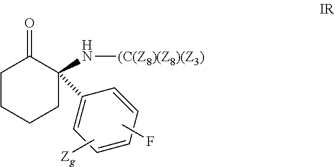

The method comprises.
(a) reacting

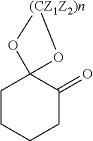

with (R)-t-butylsulfinamide in an inert solvent in the presence of Ti(OEt)₄ to form an imine of Formula VR

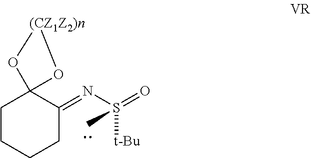

(b) reacting the imine of Formula VR with a Grignard reagent of the formula:

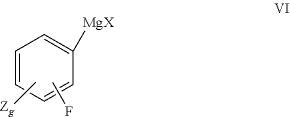

under Grignard forming conditions where X is halo to form a sulfinamide product of Formula VIIR:

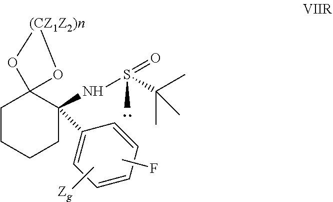

(c) reacting the compound of Formula VIIR with acid to form the corresponding amine of Formula VIIIR:
(d)

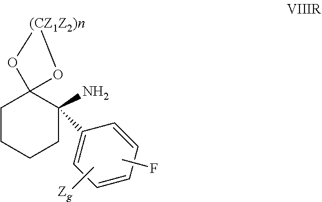

(e) reacting the amine of Formula VIIIR with an acylating agent of the formula Z₃COOXₒ wherein Xₒ is H or acid derivative thereof, or Xₒ is D under amide forming conditions to form the amide of Formula IXR:

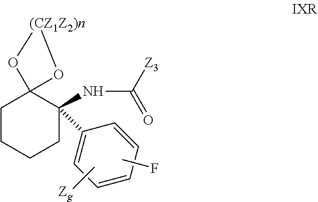

(f) reducing the acyl group (C═O) in formula IXR with sodium borohydride or deuterated sodium borohydride, both in the presence of iodine, to form the compound of Formula XR:

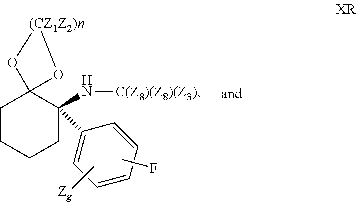

(g) deprotecting the ketal to form the compound of Formula IR wherein
Z₁ and Z₂ are independently H or C1-C6 alkyl;
n is 0, 1, 2 or 3;
X is halo;
Z₃ is hydrogen, alkyl optionally substituted with one or more halogen or C1-C6 alkoxy or aryl optionally substituted with one or more C1-C6 alkyl, C1-C6 alkoxy or halogen or Z₃ is D;
each Z is independently halogen (selected from F, Cl, Br, I), —OZ₁₁, C1-C10 alkyl, C2-C10 alkenyl, C3-C10 alkynyl having no terminal hydrogen atoms, CF₃, or OCF₃; wherein each Z₁₁ is, C1-C10 alkyl, C2-C10 alkenyl, C3-C10 alkynyl having no terminal hydrogen atoms, or aryl, and
g is 0, 1, 2, 3, or 4, and
each Z₈ is independently H or D.

In the above reaction scheme, the deuterated compounds are formed when both Z3 is D and deuterated sodium borohydride in the presence of iodine is used as the acyl reducing agent. On the other hand, if a non-deuterated acyl reducing agent is used and a non-deuterated acylating is utilized, the non-deuterated product of Formula I is produced.

In an embodiment, Z₃ is hydrogen or alkyl having 1-6 carbon atoms or D. In another embodiment, each Z is independently halogen (selected from F, Cl, Br, I), —OZ₁₁, C1-C10 alkyl, C2-C10 alkenyl, C4-C10 alkynyl having no terminal hydrogen atoms, CF₃, or OCF₃; wherein each Z₁ is C1-C10 alkyl, C2-C10 alkenyl, C4-C10 alkynyl having no terminal hydrogen atoms, or aryl.

The compounds of Formula IR can be prepared from the non-cyclic ketals similarly starting from the compound of the formula:

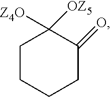

wherein the intermediates in the scheme are the corresponding non-cyclic ketals.

In another example, the S isomer of the following formula is prepared using the methodology described herein:

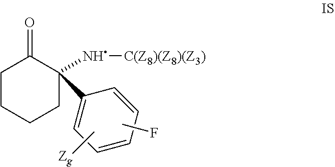

The method comprises.
(a) reacting

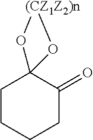

with (S)-t-butylsulfinamide in an inert solvent in the presence of Ti(OEt)₄ to form an imine of Formula VS

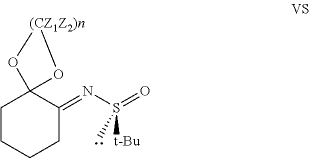

(b) reacting the imine of Formula VS with a Grignard reagent of the formula VI:

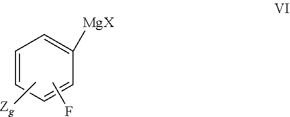

under Grignard forming conditions where X is halo to form a sulfinamide product of Formula VIIS:

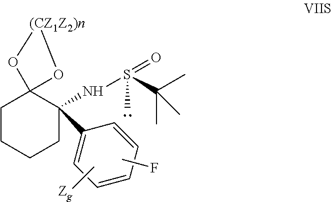

(c) reacting the compound of Formula VIIS with acid to form the corresponding amine of Formula VIIIS:

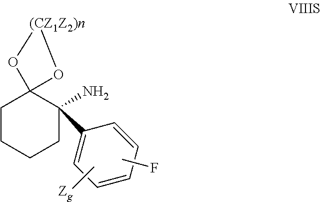

(d) reacting the amine of Formula VIIIS with an acylating agent of the formula Z₃COOXₒ wherein Xo is H or acid derivative thereof or D under amide forming conditions to form the amide of Formula IXS:

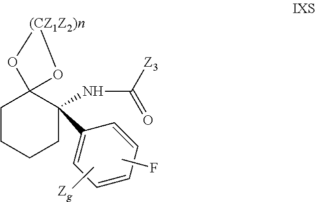

(e) reducing the acyl group (C═O) in formula IXS with sodium borohydride or deuterated sodium borohydride, both in the presence of iodine, to form the compound of Formula XS:

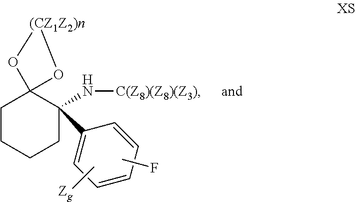

(f) deprotecting the ketal in step (e) to form the product of IS wherein
Z₁ and Z₂ are independently H or C1-C6 alkyl;
n is 0, 1, 2 or 3;
Z₃ is hydrogen, alkyl optionally substituted with one or more halogen or C1-C6 alkoxy or aryl optionally substituted with one or more C1-C6 alkyl, C1-C6 alkoxy or halogen or Z₃ is D;
each Z is independently halogen (selected from F, Cl, Br, I), —OZ₁₁, C1-C10 alkyl, C2-C10 alkenyl, C4-C10 alkynyl having no terminal hydrogen atoms, CF₃, or OCF₃; wherein each Zn is, C1-C10 alkyl, C2-C10 alkenyl, C4-C10 alkynyl having no terminal hydrogen atoms, or aryl, and
g is 0, 1, 2, 3, or 4, and
each Z₈ is independently H or D.

In the above reaction scheme, the deuterated compounds are formed when both Z₃ is D and deuterated sodium borohydride in the presence of iodine is used as the acyl reducing agent. On the other hand, if a non-deuterated acyl reducing agent is used and a non-deuterated acylating is utilized, the non-deuterated product of Formula I is produced.

In an embodiment, Z₃ is hydrogen or alkyl having 1-6 carbon atoms or D. In another embodiment, each Z is independently halogen (selected from F, Cl, Br, I), —OZ₁₁, C1-C10 alkyl, C2-C10 alkenyl, C4-C10 alkynyl having no terminal hydrogen atoms, CF₃, or OCF₃; wherein each Z₁₁ is C1-C10 alkyl, C2-C10 alkenyl, C4-C10 alkynyl having no terminal hydrogen atoms, or aryl.

The compound of Formula IS can be prepared from the non-cyclic ketals similarly starting from the compound of the formula:

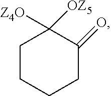

wherein the intermediates in the scheme are the corresponding non-cyclic ketals.

In an embodiment of any of the formula disclosed herein, Z₃ is H, D, or alkyl having 1 or 3 carbon atoms selected from the group consisting of —CH₃, —CH₂—CH₃, —CH₂—CH₂—CH₃, —CH—(CH₃)₂, or D. In another embodiment, Z₃ is D or H. In another embodiment, g is 0, 1, or 2. In an embodiment, g is 0. In a further embodiment, g is 1, Z is F, and the F is in the 4-position of the phenyl ring.

In an embodiment, n is 2 or 3 and Z₁ and Z₂ are independently C1-C3 alkyl or H. In a further embodiment, n is 2 and each Z₁ and Z₂ are hydrogen. In a further embodiment, n is 3 and Z₁ and Z₂ at the 4-position of the ring are both methyl and Z₁ and Z₂ at positions 3 and 5 of the ring are hydrogen. In another embodiment, n is 3 and the carbon atoms at positions 3 and 5 are unsubstituted. In another embodiment, Z₃ is hydrogen or alkyl having 1-6 carbon atoms or D. In another embodiment, each Z is independently halogen (selected from F, Cl, Br, I), —OZ₁₁, C1-C10 alkyl, C2-C10 alkenyl, C4-C10 alkynyl having no terminal hydrogen atoms, CF₃, or OCF₃; wherein each Z₁₁ is C1-C10 alkyl, C2-C10 alkenyl, C4-C10 alkynyl having no terminal hydrogen atoms, or aiyl.

A further embodiment relates to a method for forming the compound having the formula:

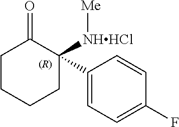

which method comprises
   (a) reacting

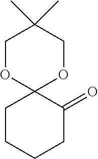

with (R)-t-butylsulfinamide in an inert solvent in the presence of Ti(OEt)₄ to form a sulfinamide having the formula

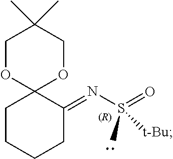

(b) reacting the product of step (a) with a Grignard reagent of the formula:

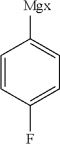

under Grignard forming conditions to form a second sulfinamide product having the formula:

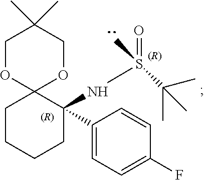

(c) reacting the product of step (b) with acid to form an amine having the formula:

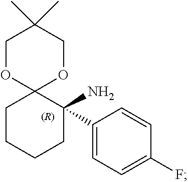

(d) reacting the amine product from step (c) with an acylating agent of formula HCOOH or acid derivative thereof under amide forming conditions to form an amide having the formula:

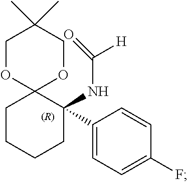

(e) reducing the acyl group (C═O) of the product in step (d) with sodium borohydride in the presence of iodine to form an amine having the formula:

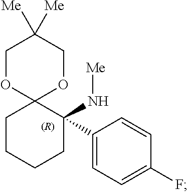

(f) deprotecting the product of step (e) in the presence of an acid selected from concentrated hydrochloric acid, concentrated hydrobromic acid, concentrated nitric acid, or concentrated sulfuric acid, with concentrated HCl, followed by base and reacting the deprotected product with HCl to form the
compound of the formula:

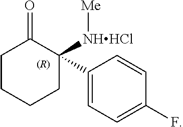

Another embodiment relates to a method for forming the compound having the formula:

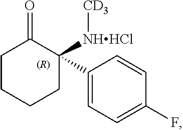

which method comprises
   (a) reacting

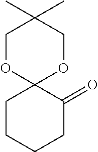

with (R)-t-butylsulfinamide in an inert solvent in the presence of Ti(OEt)₄ to form a sulfinamide having the formula

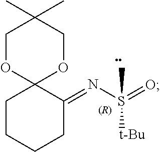

(b) reacting the product of step (a) with a Grignard reagent of the formula:

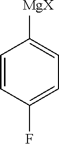

under Grignard forming conditions to form a second sulfinamide product having the formula:

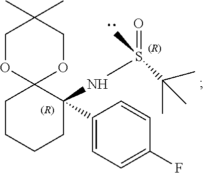

(c) reacting the product of step (b) with an acid to form an amine having the formula:

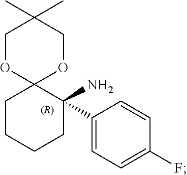

(d) reacting the amine product from step (c) with an acylating agent of formula DCOOD to form an amide product having the formula:

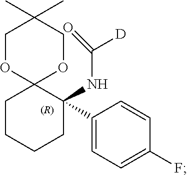

(e) reducing the acyl group (C═O) of the product in step (d) with deuterated sodium borohydride in the presence of iodine to form an amine having the formula:

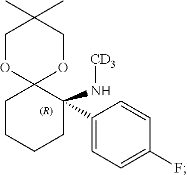

(f) deprotecting the product of step (e) in the presence of an acid selected from concentrated hydrochloric acid, concentrated hydrobromic acid, concentrated nitric acid, or concentrated sulfuric acid, reacting the resulting product with a base, and reacting the resulting product therefrom with HX₃, wherein X₃ is Br or Cl, to form the compound of the formula:

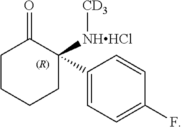

In the various processes described herein, in an embodiment, Z₆ is alkyl or phenyl which is unsubstituted or substituted with C1-C6 alkyl. In another embodiment, in the various processes described herein, each Z is independently halogen (selected from F, Cl, Br, I), —OZ₁₁, C1-C10 alkyl, C2-C10 alkenyl, C4-C10 alkynyl having no terminal hydrogen atoms, CF₃, or OCF₃; wherein each Z₁ is independently selected from C1-C10 alkyl, C2-C10 alkenyl, C3-C10 alkynyl having no terminal hydrogen atoms, and aryl which is unsubstituted or substituted with C1-C6 alkyl, C1-C6 alkoxy, or halogen. In a further embodiment herein, in the processes described herein, Z₆ is alkyl or phenyl which is unsubstituted or substituted with C1-C6 alkyl, and each Z is independently halogen (selected from F, Cl, Br, I), —OZ₁₁, C1-C10 alkyl, C2-C10 alkenyl, C4-C10 alkynyl having no terminal hydrogen atoms, CF₃, or OCF₃; wherein each Z₁ is independently selected from C1-C10 alkyl, C2-C10 alkenyl, C3-C10 alkynyl having no terminal hydrogen atoms, and aryl which is unsubstituted or substituted with C1-C6 alkyl, C1-C6 alkoxy, or halogen. In another embodiment, in the various processes described herein, Nu is Ar(Z)ₓMgX or Ar(Z)ₓLi, wherein X is halo, wherein Z is as defined herein, and x is as defined herein. In an embodiment, in the various processes described herein, Nu is Ar(Z)xMgX, wherein X is halo, Ar is phenyl, and Z and x are as defined herein. In still another embodiment, the instant process excludes the specific process for preparing [D3]-11R and [D3]-11R—HCl described in Example 2 starting from 8R to form [D3]-9R, from [D3]-9R to form [D3]-10R, from [D3]-10R to form [D3]-11R—HCl, either directly therefrom or through [D3]-1 IR whether isolated or not.

In an embodiment, the fluoro atom is on the phenyl ring, for example in the para position. In other embodiments there is no fluoro atom on the ring and the phenyl ring can have up to five substituents, as described in the earlier section of the specification.

In an embodiment, the present disclosure relates to an intermediate of the formula:

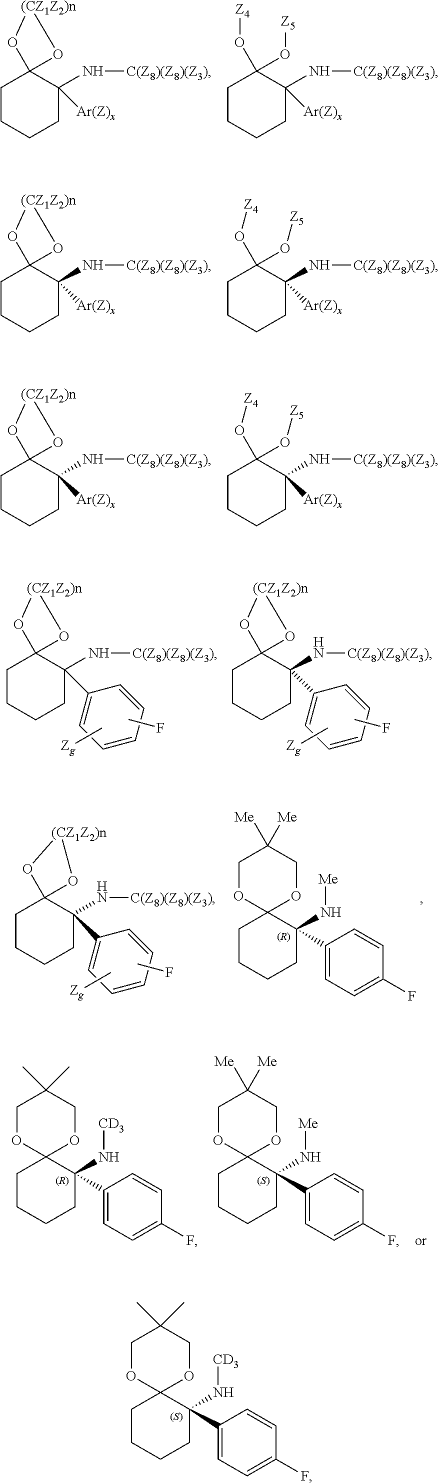

wherein Z₁, Z₂, Z₃, Z₄, Z₅, Z₈, Z, Ar, x, n and g are as defined hereinabove.

In an embodiment, the following intermediate compound may be excluded:

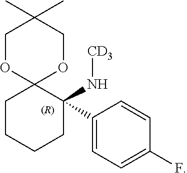

In a further embodiment, the present disclosure relates to an intermediate of the formula:

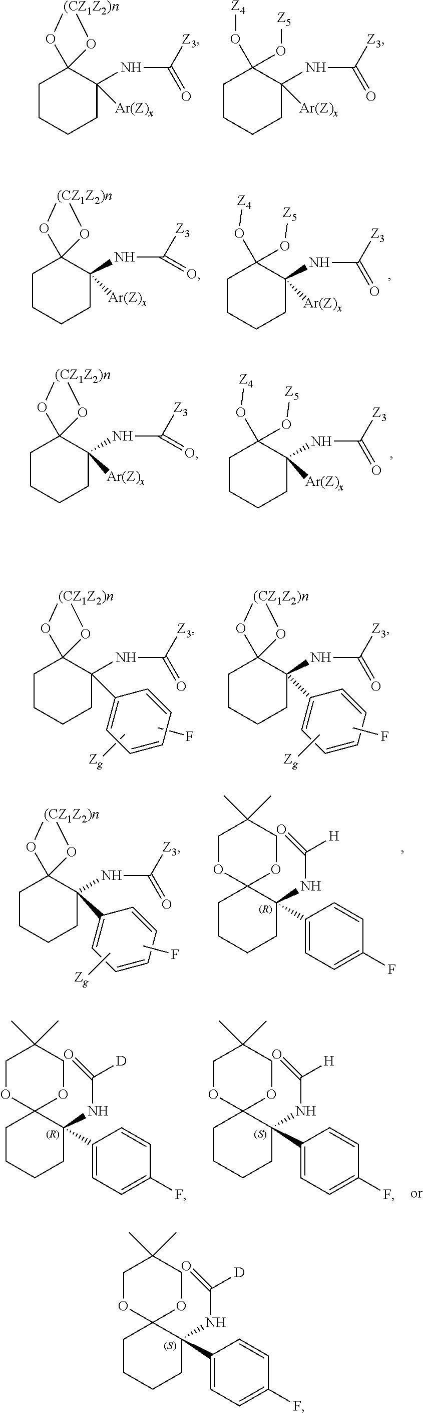

wherein Z₁, Z₂, Z₃, Z₄, Z₅, Z, Ar, x, n and g are as defined hereinabove.

In an embodiment, the following intermediate compound may be excluded:

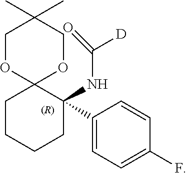

In a still further embodiment, the present disclosure relates to an intermediate compounds of the formulae:

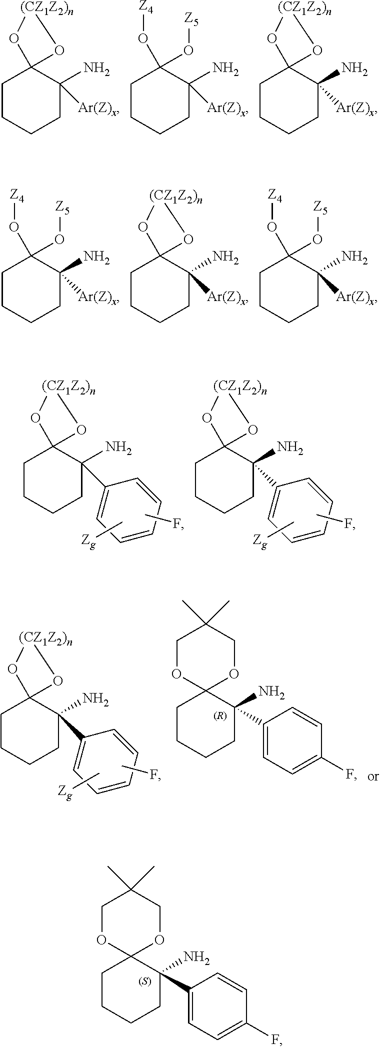

wherein Z₁, Z₂, Z₄, Z₅, Z, Ar, x, n and g are as defined hereinabove.

In an even further embodiment, the present disclosure relates to intermediates of the formula:

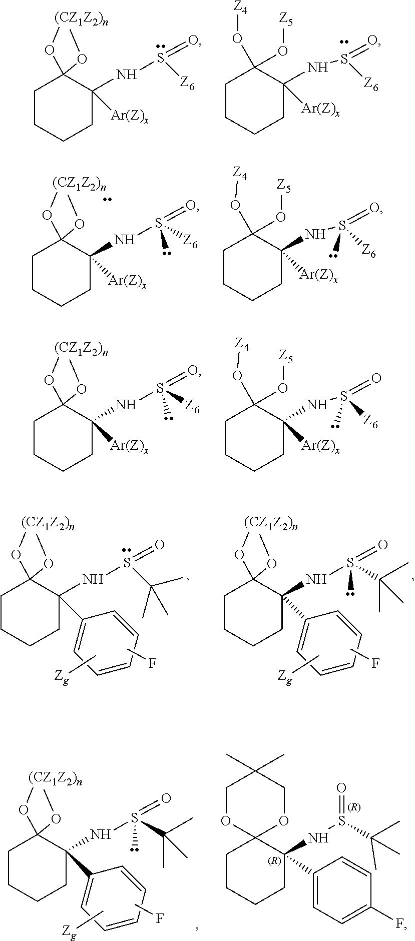

wherein Z₁, Z₂, Z₄, Z₅, Z₆, Z, Ar, x, n and g are as defined hereinabove.

In a further embodiment, the following compounds are excluded:

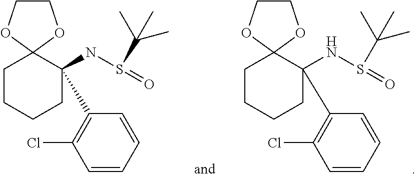 [SMILES: CC(C)(C)[S@](=O)N[C@@]1(c2ccccc2Cl)CCCCC12OCCO2 ; CC(C)(C)S(=O)NC1(c2ccccc2Cl)CCCCC12OCCO2]

In some embodiments,

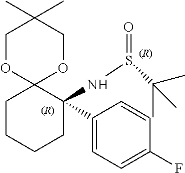

is also excluded, but not the corresponding S isomer or racemic mixture. In additional embodiments, the racemate

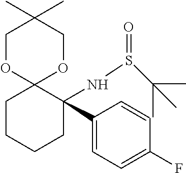 [SMILES: CC1(C)COC2(CCCC[C@]2(NS(=O)C(C)(C)C)c2ccc(F)cc2)OC1]

is excluded. In another embodiment,

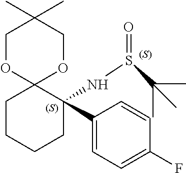

and the racemate are additionally excluded.

A further aspect of the present disclosure relates to intermediates of the formula:

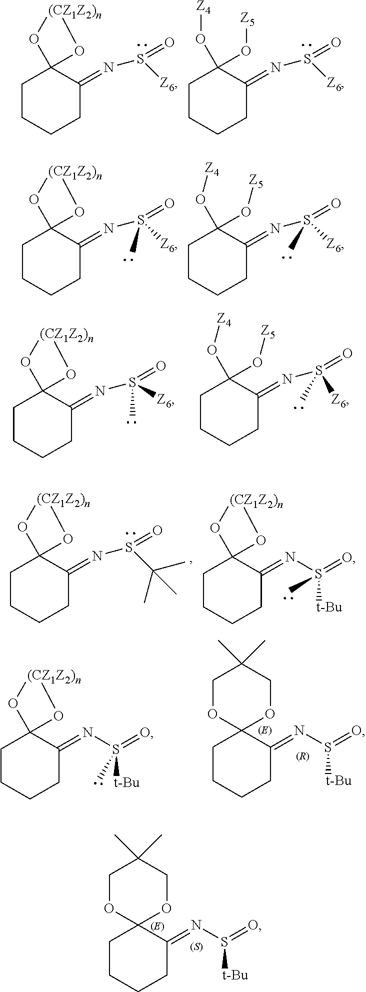

wherein Z₁, Z₂, Z₄, Z₅, Z₆, and n are as defined hereinabove.

In an embodiment, the following compounds are excluded:

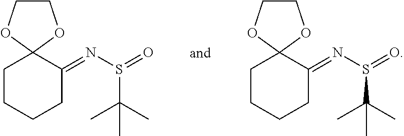 [SMILES: CC(C)(C)S(=O)/N=C1\CCCCC12OCCO2 ; "and" ; CC(C)(C)[S@](=O)/N=C1\CCCCC12OCCO2]

In some embodiments,

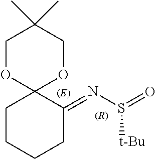

is excluded, but not the corresponding racemate or the corresponding S enantiomer. In additional embodiments, the racemate of the formula

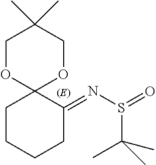 [SMILES: CC1(C)COC2(CCCC/C2=N\S(=O)C(C)(C)C)OC1]

is excluded, but not the corresponding S enantiomer. In still further embodiments, both the racemate and the corresponding S enantiomer of the formula

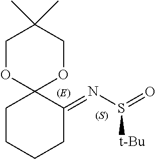

are excluded.

An even further embodiment of the present disclosure relates to an intermediate of the formula:

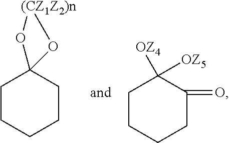

wherein Z₁, Z₂, Z₄, Z₅ and n are as defined hereinabove. In an embodiment, the following compounds are excluded:

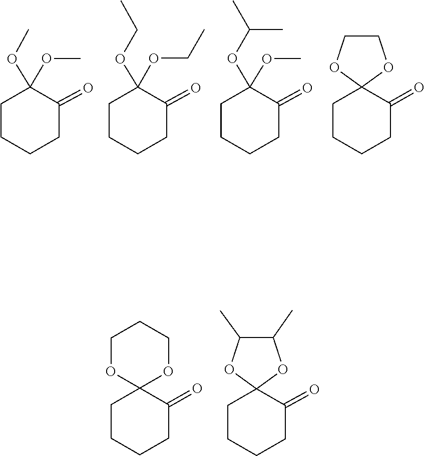

In an embodiment of the present disclosure, the present disclosure excludes intermediate compounds disclosed herein wherein Z₁ and Z₂ are hydrogen or both are methyl, and n is 2 or 3 wherein, Z₃, Z₄, Z₅, Z₆, Z₈, Z, Ar, x, and g are not present. In another embodiment, the present disclosure excludes compounds wherein Z₄ and Z₅ are both methyl or ethyl or one is methyl and the other is isopropyl, when Z₁, Z₂, Z₃, Z₅, Z₈, Z, Ar, x, n and g are not present. In another embodiment, the present disclosure excludes imine intermediate compounds wherein Z₆ is t-butyl, Z₁ and Z₂ are both hydrogen and n is 2, and when Z₁ and Z₂ are both methyl and n is 3, when Z₃, Z₄, Z₅, Z₆, Z₈, Z, Ar, x, and g are not present. In another embodiment, the present disclosure excludes compounds wherein Z₆ is t-butyl, Z₁ and Z₂ are both hydrogen and n is 2, Ar is phenyl substituted by Cl in the ortho position when the aryl ring is phenyl and z or g is 1, and Z₃, Z₄, Z₅, Z, and Z₈ are not present. In a further embodiment, when the intermediate is the R isomer, then Z₃ is other than D, F is not on the 4-position of the phenyl ring, n is other than 3 and

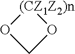

is other than

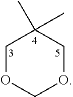

In an embodiment, the present specification excludes

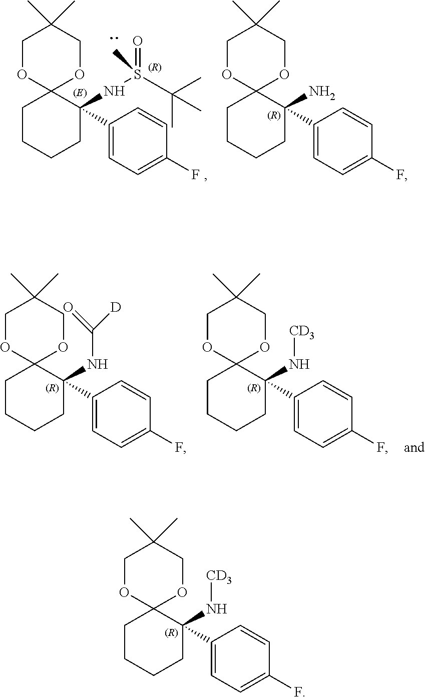

In an embodiment, the present disclosure excludes intermediate compounds identified as 5, 7R, 8R, [D₃]-9R, and [D₃]-10R. In another embodiment, the present disclosure excludes 5, 7R, 8R, [D₃]-9R, and [D3]-10R and enantiomers thereof, including stereoisomers of 7R, such as an enantiomer thereof or diastereomer thereof. In a further embodiment, the present disclosure excludes racemic mixtures comprising compounds identified as 5, 7R and 7S, 8R and 8S, [D₃]-9R and [D₃]-9S, and [D₃]-10R and [D3]-11S.

Another aspect of the present disclosure relates to a compound having the following formula:

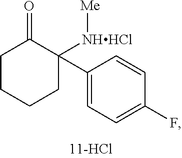

and pharmaceutical compositions comprising same. In another embodiment, the present disclosure relates to the above compound in solid form. In a further embodiment, the present specification relates to the R isomer of the hydrochloride salt described hereinabove having the formula:

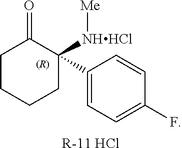

In another embodiment, the present disclosure relates to the compound R-11-HCl in solid form.

In a still further embodiment, the present disclosure relates to a compound having the formula:

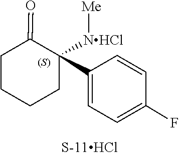

In an even further embodiment, the present disclosure relates to a compound of the formula:

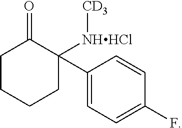

In another embodiment, the present disclosure relates to a compound of the formula:

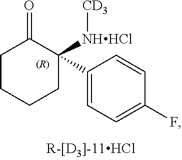

while in another embodiment, the present disclosure relates to a compound of the formula:

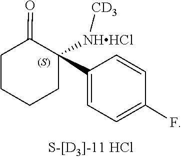

The compounds described herein which are deuterated will be referenced herein as the deuterated analogs.

The compounds disclosed herein of Formula I, including IR and IS, the deuterated analogs, or pharmaceutically acceptable salts thereof, are useful for treating a psychiatric disorder. The utility is the same as described in U.S. Pat. No. 11,344,510, the contents of which are incorporated by reference.

More specifically, in another aspect, provided herein is a method of treating depression or anxious depression in a subject in need thereof, the method comprising administering to the subject in need thereof an effective amount of a compound of Formula I, IR, IS, the deuterated analogs, or pharmaceutically acceptable salts thereof disclosed herein.

In some embodiments, the compounds of Formula I, IR, IS, the deuterated analogs, or pharmaceutically acceptable salts thereof are orally administered. For example, the compounds of Formula 1, IR, IS, the deuterated analogs, or pharmaceutically acceptable salts thereof are useful in treating a psychiatric disorder comprising administering to a subject in need thereof a therapeutically effective amount of a compound, or composition disclosed herein. Contemplated psychiatric disorders may include Depressive Disorders, e.g., Major Depressive Disorder, Persistent Depressive Disorder, Postpartum Depression, Premenstrual Dysphoric Disorder, Seasonal Affective Disorder, Psychotic Depression, Disruptive Mood Dysregulation Disorder, Substance/Medication-Induced Depressive Disorder, and Depressive Disorder Due to Another Medical Condition. The compounds of Formula I, IR, IS are useful for treating refractory depression, e.g., patients suffering from a depressive disorder that does not, and/or has not, responded to adequate courses of at least one, or at least two, other antidepressant compounds or therapeutics. As used herein “depressive disorder” encompasses refractory depression. In some embodiments, the compounds of Formula I, IR, IS, the deuterated analogs, and pharmaceutically acceptable salts thereof may be used to treat a psychiatric disorder including Bipolar and Related Disorders, e.g., Bipolar I Disorder, Bipolar II Disorder, Cyclothymic Disorder, Substance/Medication-Induced Bipolar and Related Disorder, and Bipolar and Related Disorders due to another medical condition. In some embodiments, the compounds of Formula I, IR, IS, the deuterated analogs, or pharmaceutically acceptable salts thereof may be used to treat a psychiatric disorder including Substance-Related Disorders, e.g., preventing a substance use craving, diminishing a substance use craving, and/or facilitating substance use cessation or withdrawal. Substance use disorders involve abuse of psychoactive compounds such as alcohol, caffeine, cannabis, inhalants, opioids, sedatives, hypnotics, anxiolytics, stimulants, nicotine, and tobacco. As used herein “substance” or “substances” are psychoactive compounds which can be addictive such as alcohol, caffeine, cannabis, hallucinogens, inhalants, opioids, sedatives, hypnotics, anxiolytics, stimulants, nicotine, and tobacco. For example, the methods and compositions may be used to facilitate smoking cessation or cessation of opioid use.

In some embodiments, the compounds of Formula I, IR, IS, the deuterated analogs, or pharmaceutically acceptable salts thereof may be used to treat a psychiatric disorder including Anxiety Disorders, e.g., Separation Anxiety Disorder, Selective Mutism, Specific Phobia, Social Anxiety Disorder (Social Phobia), Panic Disorder, Panic Attack, Agoraphobia, Generalized Anxiety Disorder, Substance/Medication-Induced Anxiety Disorder, and Anxiety Disorder Due to Another Medical Condition. In embodiments, the compounds of Formula I, IR, IS, the deuterated analogs, or pharmaceutically acceptable salts thereof may be used to treat a psychiatric disorder including Obsessive-Compulsive and Related Disorders, e.g., Obsessive-Compulsive Disorder, Body Dysmorphic Disorder, Hoarding Disorder, Trichotillomania (Hair-Pulling Disorder), Excoriation (Skin-Picking) Disorder, Substance/Medication-Induced Obsessive-Compulsive, and Related Disorder, and Obsessive-Compulsive and Related Disorder Due to Another Medical Condition. In some embodiments, the compounds of Formula I, IR, IS, the deuterated analogs, or pharmaceutically acceptable salts thereof may be used to treat a psychiatric disorder including Trauma- and Stressor-Related Disorders, e.g., Reactive Attachment Disorder, Disinhibited Social Engagement Disorder, Posttraumatic Stress Disorder, Acute and Stress Disorder, and Adjustment Disorders. In some embodiments, the compounds of Formula I, IR, IS, the deuterated analogs, or pharmaceutically acceptable salts thereof may be used to treat a psychiatric disorder including Feeding and Eating Disorders, e.g., Anorexia Nervosa, Bulimia Nervosa, Binge-Eating Disorder, Pica, Rumination Disorder, and Avoidant/Restrictive Food Intake Disorder.

Further, in some embodiments, the compounds of Formula I, JR, IS, the deuterated analogs, or pharmaceutically acceptable salts thereof may be used to treat a psychiatric disorder including Neurocognitive Disorders, e.g., Delirium, Major Neurocognitive Disorder, Mild Neurocognitive Disorder, Major or Mild Neurocognitive Disorder Due to Alzheimer's Disease, Major or Mild Frontotemporal Neurocognitive Disorder, Major or Mild Neurocognitive Disorder With Lewy Bodies, Major or Mild Vascular Neurocognitive Disorder, Major or Mild Neurocognitive Disorder Due to Traumatic Brain Injury, Substance/Medication-Induced Major or Mild Neurocognitive Disorder, Major or Mild Neurocognitive Disorder Due to HIV Infection, Major or Mild Neurocognitive Disorder Due to Prion Disease, Major or Mild Neurocognitive Disorder Due to Parkinson's Disease, Major or Mild Neurocognitive Disorder Due to Huntington's Disease, Major or Mild Neurocognitive Disorder Due to Another Medical Condition, and Major or Mild Neurocognitive Disorder Due to Multiple Etiologies. Moreover, In some embodiments, the compounds of Formula I, IR, IS, the deuterated analogs, or pharmaceutically acceptable salts thereof may be used to treat a psychiatric disorder including Neurodevelopmental Disorders, e.g., Autism Spectrum Disorder, Attention-Deficit/Hyperactivity Disorder, Stereotypic Movement Disorder, Tic Disorders, Tourette's Disorder, Persistent (Chronic) Motor or Vocal Tic Disorder, and Provisional Tic Disorder. Further, in some embodiments, the compounds of Formula I, IR, IS, the deuterated analogs, or pharmaceutically acceptable salts thereof may be used to treat a psychiatric disorder including Personality Disorders, e.g., Borderline Personality Disorder.

In addition, in some embodiments, the compounds of Formula I, IR, IS, the deuterated analogs, or pharmaceutically acceptable salts thereof may be used to treat a psychiatric disorder including Sexual Dysfunctions, e.g., Delayed Ejaculation, Erectile Disorder, Female Orgasmic Disorder, Female Sexual Interest/Arousal Disorder, Genito-Pelvic Pain/Penetration Disorder, Male Hypoactive Sexual Desire Disorder, Premature (Early) Ejaculation, and Substance/Medication-Induced Sexual Dysfunction. In some embodiments the compounds of Formula I, IR, IS, the deuterated analogs, or pharmaceutically acceptable salts thereof may be used to treat a psychiatric disorder including Gender Dysphoria, e.g., Gender Dysphoria.

The terms “effective amount” or “therapeutically effective amount” refer to an amount of a compound of Formula I, IR, IS, the deuterated analogs or pharmaceutically acceptable salts thereof that are effective to achieve a particular pharmacological and/or physiologic effect including but not limited to reducing the frequency or severity of sadness or lethargy, depressed mood, anxious or sad feelings, diminished interest in all or nearly all activities, significant increased or decreased appetite leading to weight gain or weight loss, insomnia, irritability, fatigue, feelings of worthlessness, feelings of helplessness, inability to concentrate, and recurrent thoughts of death or suicide, or to provide a desired pharmacologic and/or physiologic effect, for example, reducing, inhibiting, or reversing one or more of the underlying pathophysiological mechanisms underlying the neurological dysfunction, modulating dopamine levels or signaling, modulating serotonin levels or signaling, modulating norepinephrine levels or signaling, modulating glutamate or GABA levels or signaling, modulating synaptic connectivity or neurogenesis in certain brain regions, or a combination thereof.

The term “therapeutic index” used in reference to any compound of Formula I, IR, IS, the deuterated analogs and/or pharmaceutically acceptable salts thereof and associated therapeutic effects and side effects refers to the ratio of the dose of said compound required to induce a particular negative side effect to the dose of said compound required to induce the desired therapeutic effect.

In some embodiments, methods of using compounds of Formula I, IR, IS, the deuterated analogs, or pharmaceutically acceptable salts thereof include treating a psychiatric disorder by administering to a subject in need thereof a pharmaceutical composition including about 0.01 mg to about 400 mg of a compound disclosed herein. In some embodiments, doses may be, e.g., in the range of about 0.1 to 300 mg, 0.1 to 250 mg, 0.1 to 200 mg, 0.1 to 150 mg, 0.1 to 100 mg, 0.1 to 75 mg, 0.1 to 50 mg, 0.1 to 25 mg, 0.1 to 20 mg, 0.1 to 15 mg, 0.1 to 10 mg, 0.1 to 5 mg, 0.1 to 1 mg, 10 to 300 mg, 10 to 250 mg, 10 to 200 mg, 10 to 150 mg, 10 to 100 mg, 10 to 50 mg, 10 to 25 mg, 10 to 15 mg, 20 to 300 mg, 20 to 250 mg, 20 to 200 mg, 20 to 150 mg, 20 to 100 mg, 20 to 50 mg, 50 to 300 mg, 50 to 250 mg, 50 to 200 mg, 50 to 150 mg, 50 to 100 mg, 100 to 300 mg, 100 to 250 mg, 100 to 200 mg, with doses of, e.g., about 0.25 mg, 0.5 mg, 0.75 mg, 1 mg, 1.25 mg, 1.5 mg, 1.75 mg, 2.0 mg, 2.5 mg, 3.0 mg, 3.5 mg, 4.0 mg, 4.5 mg, 5 mg, 10 mg, 15 mg, 20 mg, 25 mg, 30, mg, 35 mg, 40 mg, 45 mg, 50 mg, 75 mg, 100 mg, 125 mg, 150 mg, 175 mg, 200 mg, 225 mg, 250 mg, 275 mg, 300 mg, and 400 mg being examples.

In some embodiments, dosages may include amounts of a compound of Formula I, IR, IS, the deuterated analogs, or a pharmaceutically acceptable salt thereof in the range of about, e.g., 1 mg to 200 mg, 1 mg to 100 mg, 1 mg to 50 mg, 1 mg to 40 mg, 1 mg to 30 mg, 1 mg to 20 mg, 1 mg to 15 mg, 0.01 mg to 10 mg, 0.1 mg to 15 mg, 0.15 mg to 12.5 mg, or 0.2 mg to 10 mg, with doses of 0.1 mg, 0.2 mg, 0.3 mg, 0.4 mg, 0.5 mg, 0.6 mg, 0.7 mg, 0.8 mg, 0.9 mg, 1.5 mg, 1.0 mg, 1.75 mg, 2 mg, 2.5 mg, 2.75 mg, 3 mg, 3.5 mg, 3.75 mg, 4 mg, 4.5 mg, 4.75 mg, 5 mg, 5.5 mg, 6 mg, 6.5 mg, 7 mg, 7.5 mg, 8 mg, 8.5 mg, 9 mg, 10 mg, 11 mg, 12 mg, 15 mg, 20 mg, 25 mg, 30 mg, 35 mg, 40 mg, 45 mg, 50 mg, 60 mg, 75 mg, 80 mg, 90 mg, 100 mg, 125 mg, 150 mg, and 200 mg being specific examples of doses.

Typically, dosages of a compound of Formula I, IR, IS, the deuterated analogs disclosed herein or a pharmaceutically acceptable salt thereof is administered once, twice, three or four times daily, every other day, every three days, once weekly, or once a month to a patient in need thereof. In some embodiments, the dosage is about, e.g., 1-400 mg/day, or 1-300 mg/day, or 1-250 mg/day, or 1-200 mg/day, for example 300 mg/day, 250 mg/day, 200 mg/day, 150 mg/day, 100 mg/day, 75 mg/day, 50 mg/day, 25 mg/day, 20 mg/day, 10 mg/day, 5 mg/day, or 1 mg/day.

In some embodiments, pharmaceutical compositions for parenteral or inhalation, e.g., a spray or mist of a compound of Formula I, IR, IS, the deuterated analogs or a pharmaceutically acceptable salt thereof include a concentration of about 0.005 mg/mL to about 500 mg/mL. In some embodiments, the compositions include a compound of Formula I, IR, IS, the deuterated analogs or a pharmaceutically acceptable salt thereof at a concentration of, e.g., about 0.05 mg/mL to about 50 mg/mL, about 0.05 mg/mL to about 100 mg/mL, about 0.005 mg/mL to about 500 mg/mL, about 0.1 mg/mL to about 50 mg/mL, about 0.1 mg/mL to about 10 mg/mL, about 0.05 mg/mL to about 25 mg/mL, about 0.05 mg/mL to about 10 mg/mL, about 0.05 mg/mL to about 5 mg/mL, or about 0.05 mg/mL to about 1 mg/mL.

In some embodiments, the composition includes a compound of Formula I, IR, IS, the deuterated analogs or a pharmaceutically acceptable salt thereof at a concentration of, e.g., about 0.05 mg/mL to about 15 mg/mL, about 0.5 mg/mL to about 10 mg/mL, about 0.25 mg/mL to about 5 mg/mL, about 0.5 mg/mL to about 7 mg/mL, about 1 mg/mL to about 10 mg/mL, about 5 mg/mL to about 10 mg/mL, about 5 mg/mL to about 15 mg/mL, about 5 mg/mL to 25 mg/mL, about 5 mg/mL to 50 mg/mL, or about 10 mg/mL to 100 mg/mL. In some embodiments, the pharmaceutical compositions are formulated as a total volume of about, e.g., 10 mL, 20 mL, 25 mL, 50 mL, 100 mL, 200 mL, 250 mL, or 500 mL.

Typically, dosages may be administered to a subject once, twice, three or four times daily, every other day, every three days, twice weekly, once weekly, twice monthly, or once monthly. In some embodiments, a compound of Formula I, IR, IS, the deuterated analogs or pharmaceutically acceptable salts thereof is administered to a subject once in the morning, or once in the evening. In some embodiments, these aforementioned compounds may be administered to a subject once in the morning, and once in the evening. In some embodiments, a compound of Formula I, IR, IS, the deuterated analogs or pharmaceutically acceptable salts thereof is administered to a subject three times a day (e.g., at breakfast, lunch, and dinner), at a dose, e.g., of 50 mg/administration (e.g., 150 mg/day).

In some embodiments, a compound of Formula I, IR, IS, the deuterated analogs or pharmaceutically acceptable salts thereof is administered to a subject at a dose of 25 mg/day in one or more doses. In some embodiments, a compound of Formula I, IR, IS, the deuterated analogs or pharmaceutically acceptable salts thereof is administered to a subject at a dose of 50 mg/day in one or more doses. In some embodiments, a compound of Formula I, IR, IS, the deuterated analogs or pharmaceutically acceptable salts thereof is administered to a subject at a dose of 75 mg/day in one or more doses. In some embodiments, a compound of Formula I, IR, IS, the deuterated analogs or pharmaceutically acceptable salts thereof is administered to a subject at a dose of 100 mg/day in one or more doses. In some embodiments, a compound of Formula I, IR, IS, the deuterated analogs or pharmaceutically acceptable salts thereof is administered to a subject at a dose of 150 mg/day in one or more doses. In some embodiments, a compound of Formula I, IR, IS, the deuterated analogs or pharmaceutically acceptable salts thereof is administered to a subject at a dose of 200 mg/day in one or more doses. In some embodiments, a compound of Formula I, IR, IS, the deuterated analogs or pharmaceutically acceptable salts thereof is administered to a subject at a dose of 250 mg/day in one or more doses.

In some embodiments, the dosage of a compound of Formula I, IR, IS, the deuterated analogs or pharmaceutically acceptable salts thereof is 0.01-100 mg/kg, 0.5-50 mg/kg, 0.5-10 mg/kg, or 25-50 mg/kg once, twice, three times or four times daily. For example, in some embodiments, the dosage is 0.1 mg/kg, 0.25 mg/kg, 0.5 mg/kg, 1 mg/kg, 5 mg/kg, 7.5 mg/kg, or 10 mg/kg once, twice, three times or four times daily. In some embodiments, a subject is administered a total daily dose of 0.01 mg to 500 mg of a compound of Formula I, IR, IS, the deuterated analogs or pharmaceutically acceptable salts thereof once, twice, three times, or four times daily. In some embodiments, the total amount administered to a subject in 24-hour period is, e.g., 5 mg, 10 mg, 20 mg, 25 mg, 30 mg, 35 mg, 40 mg, 45 mg, 50 mg, 60 mg, 75 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg. In some embodiments, the subject may be started at a low dose and the dosage is escalated. In some embodiments, the subject may be started at a high dose and the dosage is decreased.

In some embodiments, a compound of Formula I, IR, IS, the deuterated analogs or pharmaceutically acceptable salts thereof is administered to a patient under the supervision of a healthcare provider.

In some embodiments, a compound of Formula I, IR, IS, the deuterated analogs or pharmaceutically acceptable salts thereof is administered to a patient under the supervision of a healthcare provider at a clinic specializing in the delivery of psychoactive treatments.

In some embodiments, a compound of Formula I, IR, IS, the deuterated analogs or pharmaceutically acceptable salts thereof is administered to a patient under the supervision of a healthcare provider at a dose intended to induce a psychedelic experience in the subject.

In some embodiments, the administration to a patient under the supervision of a healthcare provider occurs periodically in order to maintain a therapeutic effect in the patient, e.g., every three days, twice weekly, once weekly, twice monthly, once monthly, thrice yearly, twice yearly, or once yearly.

In some embodiments, a compound of Formula I, IR, IS, the deuterated analogs or pharmaceutically acceptable salts thereof is administered by a patient on their own at home or otherwise away from the supervision of a healthcare provider.

In some embodiments, the administration by a patient on their own occurs periodically in order to maintain a therapeutic effect in the patient, e.g., daily, every other day, every three days, twice weekly, once weekly, twice monthly, or once monthly.

In some embodiments, a compound of Formula I, IR, IS, the deuterated analogs or pharmaceutically acceptable salts thereof may be administered at specified intervals. For example, during treatment a patient may be administered a compound of Formula I, IR, IS, the deuterated analogs or pharmaceutically acceptable salts thereof at intervals of every, e.g., 1 year, 6 months, 90 days, 60 days, 30 days, 14 days, 7 days, 3 days, 24 hours, 12 hours, 8 hours, 6 hours, 5 hours, 4 hours, 3 hours, 2.5 hours, 2.25 hours, 2 hours, 1.75 hours, 1.5 hours, 1.25 hours, 1 hour, 0.75 hour, 0.5 hour, or 0.25 hour. In some embodiments, a compound of Formula I, IR, IS, or the deuterated analogs are in the form of a pharmaceutically acceptable salt.

In some embodiments, a pharmaceutical composition comprises one or more of the compounds of Formula I, IR, IS, the deuterated analogs or pharmaceutically acceptable salts thereof. In some embodiments, a salt of the compound of Formula I, IR, and/or IS, the deuterated analogs, or pharmaceutically acceptable salts thereof is used in any of the methods, uses, or compositions described herein.

In some embodiments, a pharmaceutically acceptable salt of the compounds of Formula I, IR, and/or IS or the deuterated analogs is used in any of the methods, uses, or compositions described herein.

The term “treatment” as used herein means the management and care of a patient for the purpose of combating a disease, disorder, or condition. The term is intended to include the delaying of the progression of the disease, disorder or condition, the alleviation or relief of symptoms and complications, and/or the cure or elimination of the disease, disorder, or condition. The patient to be treated is preferably a mammal, in particular a human being.

The present disclosure thus also relates to pharmaceutical compositions comprising a compound of Formula I, IR, IS, the deuterated analogs or pharmaceutically acceptable salts thereof in admixture with pharmaceutically acceptable auxiliaries, and optionally other therapeutic agents.

The auxiliaries must be “acceptable” in the sense of being compatible with the other ingredients of the composition and not deleterious to the recipients thereof.

Pharmaceutical compositions include those suitable for oral, rectal, nasal, topical (including transdermal, buccal, and sublingual), vaginal or parenteral (including subcutaneous, intramuscular, intravenous, and intradermal) administration or administration via an implant. The compositions may be prepared by any method well known in the art of pharmacy.

Such methods include the step of bringing in association compounds of Formula I, IR, IS, the deuterated analogs or pharmaceutically acceptable salts thereof or combinations thereof with any auxiliary agent. The auxiliary agent(s), also include accessory ingredient(s), include those conventional in the art, such as carriers, fillers, binders, diluents, disintegrants, lubricants, colorants, flavoring agents, anti-oxidants, and wetting agents. Such auxiliary agents are suitably selected with respect to the intended form and route of administration and as consistent with conventional pharmaceutical practices.

Pharmaceutical compositions suitable for oral administration may be presented as discrete dosage units such as pills, tablets, dragées or capsules, or as a powder or granules, or as a solution or suspension. The active ingredient comprised of compounds of Formula I, IR, IS, the deuterated analogs or pharmaceutically acceptable salts thereof may also be presented as a bolus or paste. The compositions can further be processed into a suppository or enema for rectal administration.

Tablets may contain the active ingredient compounds comprised of Formula I, IR, IS, the deuterated analogs or pharmaceutically acceptable salt thereof and suitable binders, lubricants, disintegrating agents, coloring agents, flavoring agents, flow-inducing agents, and melting agents. Gelatin capsules may contain the active ingredient compounds comprised of Formula I, IR, IS, the deuterated analogs or pharmaceutically acceptable salts thereof and powdered carriers, such as lactose, starch, cellulose derivatives, magnesium stearate, stearic acid, and the like. Similar diluents can be used to make compressed tablets. Compressed tablets can be sugar coated or film coated to mask any unpleasant taste and protect the tablet from the atmosphere, or enteric coated for selective disintegration in the gastrointestinal tract. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, and the like. Suitable binders include starch, gelatin, natural sugars such as glucose or beta-lactose, com sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

For oral administration in liquid dosage form, the compounds of Formula I, IR, IS, the deuterated analogs or pharmaceutically acceptable salts thereof are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols, or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Liquid dosage forms for oral administration can contain coloring and flavoring to increase patient acceptance.

For parenteral administration, suitable compositions include aqueous and non-aqueous sterile solutions comprised of compounds of Formula I, IR, IS, the deuterated analogs or pharmaceutically acceptable salts thereof disclosed herein. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. The compositions may be presented in unit-dose or multi-dose containers, for example sealed vials and ampoules, and may be stored in a freeze-dried (lyophilized) condition requiring only the addition of sterile liquid carrier, for example water, prior to use. For transdermal administration, e.g., gels, patches or sprays can be contemplated.

Compositions or formulations suitable for pulmonary administration e.g., by nasal inhalation, include fine dusts or mists which may be generated by means of metered dose pressurized aerosols, nebulizers, or insufflators. Parenteral and intravenous forms may also include minerals and other materials to make them compatible with the type of injection or delivery system chosen.

The compounds used in the method of the present disclosure may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds of Formula I, IR, IS, the deuterated analogs or pharmaceutically acceptable salt thereof used in the method of the present disclosure may also be coupled to soluble polymers as targetable drug carriers or as prodrugs. Such polymers include polyvinylpyrrolidone, pyran copolymer, polyhydroxylpropylmethacrylamide-phenol, polyhydroxyethylaspartamide-phenol, or polyethyleneoxide-polylysine substituted with palmitoyl residues. Furthermore, the compounds of Formula I, IR, IS, or pharmaceutically acceptable salts thereof may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Pharmaceutical compositions herein may be provided with immediate release, delayed release, extended release, or modified release profiles. In some embodiments, pharmaceutical compositions with different drug release profiles may be combined to create a two-phase or three-phase release profile. For example, pharmaceutical compositions may be provided with an immediate release and an extended-release profile. Such composition may be provided as pulsatile formulations, multilayer tablets, or capsules containing tablets, beads, granules, etc.

Pharmaceutical compositions herein may be provided with abuse deterrent features by techniques know in the art, for example, by making a tablet that is difficult to crush or to dissolve in water.

The pharmaceutical composition, as hereinbefore described, may be in combination with packaging material, including instructions for the use of the composition for a use as hereinbefore described.

The exact dose and regimen of administration of the composition comprised of compounds of Formula I, IR, IS, the deuterated analogs or pharmaceutically acceptable salts thereof will necessarily be dependent upon the type and magnitude of the therapeutic or nutritional effect to be achieved and may vary depending on factors such as the particular compound, formula, route of administration, or age and condition of the individual subject to whom the composition is to be administered.

Furthermore, in some embodiments, a pharmaceutical composition disclosed herein may include a single enantiomer, diastereomer or structural isomer of a compound of Formula I, IR, IS, the deuterated analogs or pharmaceutically acceptable salts thereof. In other embodiments, a pharmaceutical composition disclosed herein may include a mixture of at least one single enantiomer, diastereomer or structural isomer of a compound of Formula I, IR, IS, the deuterated analogs or pharmaceutically acceptable salts thereof together with another enantiomer, diastereomer or structural isomer of a compound of Formula I, IR, IS, the deuterated analogs or pharmaceutically acceptable salt thereof. In further embodiments, said mixture is a racemic mixture. In other embodiments, said mixture is a non-racemic mixture (wherein one enantiomer or diastereomer is enriched in said non-racemic mixture). In some embodiments, the compounds of Formula I, JR, IS, or pharmaceutically acceptable salts thereof are substantially pure or are enantiomerically pure or both.

The compounds of Formula I, IR, IS, the deuterated analogs or pharmaceutically acceptable salts thereof may be administered in various forms, including those detailed herein. The treatment with the compound of Formula I, IR, IS, the deuterated analogs or pharmaceutically acceptable salts thereof may be a component of a combination therapy or an adjunct therapy, i.e., the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

The following examples further illustrate the teachings of the present disclosure. The following examples are exemplary of the teachings herein. Even though this is an exemplification, the teachings for preparing the compound described hereinbelow can be prepared using the teachings herein and is not intended to be limited to the following exemplification.

In the following examples, the following abbreviations are used:
p-TSA: Para-toluenesulfonic acid
MTBE: Methyl tert-butyl ether
EDTE: Ethylenediamine tetraethanol
V: Volume
Q-NMR: Quantitative NMR
THF: Tetrahydrofuran
HPLC: High-performance liquid chromatography
NLT: Not less than
TLC: Thin layer chromatography
DCM: Dichloromethane
DMSO: Dimethyl sulfoxide
ETOAc: Ethyl Acetate
IPA: Isopropyl alcohol
ETOH: Ethanol
NaHCO₃: Sodium bicarbonate
rt: Room Temperature (20-25° C.)
NMP: n-Methyl-2-pyrrolidone

In the following schemes, compound 5R is a species of the genus of the compound of Formula VR, compound 7R is a species of the compound of Formula VIIR, compound 8R is a species of the compound of VIIIR, compound 9R is a species compound of IXR, compound 10R is a species of XR, and compound 11R is a species of IR, [D3]-11R is a species of the genus IR, corresponding to R-[D3]-11 wherein Z₃ and Z₈ are both D, [D3]-10R is a species of genus XR, wherein Z₃ and Z₈ are both D, and [D]-9R is a species of genus IXR, wherein Z₃ is D. Similarly, 5S is a species of the genus of Formula VS, compound 7S is a species of Formula VIIS, compound 8S is a species of VIIIS, compound 9S is a species of IXS, compound 10S is a species of XS, and compound 11S is a species of IS, [D3]-11S is a species of the genus IS, corresponding to R-[D3]-11 wherein Z₃ and Z₈ are both D, [D3]-10S is a species of genus XS, wherein Z₃ and Z₈ are both D, and [D]-9S is a species of genus IXS, wherein Z₃ is D.

### Example 1

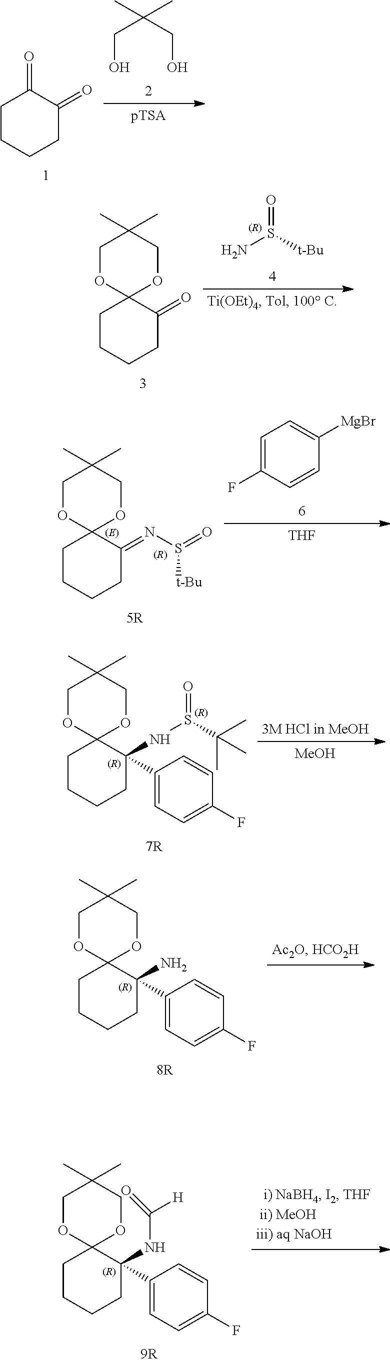

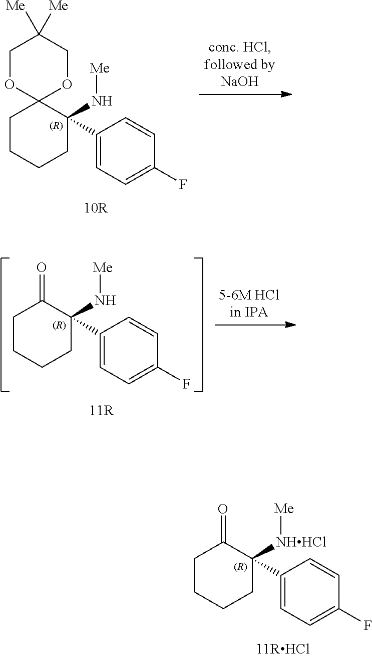

### General Procedure for the Synthesis of 3,3-dimethyl-1,5-dioxaspiro[5.5]undecane-7-one, 3

A 1000 mL jacketed reactor equipped with an over-head stirrer and a Dean-Stark apparatus was charged with 1,2-cyclohexanedione (50.0 g, 428 mmol), 2,2-dimethylpropane-1,3-diol (54.0 g, 514 mmol), p-TSA (1.66 g, 8.6 mmol) and cyclohexane (200 mL) and the resulted suspension was heated at reflux for 3 h to obtain a complete conversion of the starting material. It was then cooled to room temperature and charged with 1N NaOH(aq) followed by MTBE and stirred. The phases were separated, and the aqueous phase was further extracted with MTBE and the combined organics were washed once with 10% brine and concentrated. The mixture was azeotroped once with toluene to obtain the 113 g (Q-NMR assay: 66%, yield 87.6%). The crude product was taken to the next step without further purification. ¹H NMR (400 MHz, CDCl₃) δ 3.55 (d, J=11.1 Hz, 1H), 3.32 (d, J=11.1 Hz, 1H), 2.38-2.35 (m, 2H), 1.8-1.79 (m, 2H), 1.67-1.63 (m, 4H), 1.06 (s, 3H), 0.55 (s, 3H).

### General Procedure for the Synthesis of (R, E)-N-(3,3-dimethyl-1,5-dioxaspiro[5.5]undecan-7-ylidene)-2-methylpropane-2-sulfinamide, 5R

A round bottom flask equipped with an overhead stirrer was charged compound 3 (33.3 g, 60% wt. %, 0.101 mol), t-Bu-Sulfinamide (14.62 g, 0.121 mol), toluene (80 mL) and Ti(OEt₄) (25.31 mL, 0.121 mol) at room temperature. The mixture was heated at 80 C for 5-6 h followed by cooling to room temperature to obtain a dark solution. To this solution was added EDTE (47.5 g), and the mixture was heated at 55 C for 60 minutes followed by cooling to room temperature. To the above solution at was added 12% NaCl (aq) stirred for about 5 mins and allowed to settle. Phases were separated, and the aqueous phase was re-extracted twice with toluene. The combined organics were washed once with water. The organic phase was filtered through a plug of activated charcoal and SiO₂ and concentrated to obtain the crude product as yellow-orange semi solid (18.9 g net product by NMR wt %, 63%). The product crystalized out as off-white solid upon standing, which was filtered and carried to the next step. ¹H NMR (400 MHz, CDCl₃) δ 3.83 (d, J=11.0 Hz, 1H), 3.72 (d, J=11.0 Hz, 1H), 3.44-3.38 (m, 2H), 3.13-3.07 (m, 1H), 2.89-2.83 (m, 1H), 1.98-1.85 (m, 1H), 1.81-1.71 (m, 1H), 1.31 (s, 9H), 1.21 (s, 3H), 0.72 (s, 3H).

### General Procedure for the Synthesis of (R)—N—((R)-7-(4-fluorophenyl)-3,3-dimethyl-1,5-dioxaspiro[5.5]undecan-7-yl)-2-methylpropane-2-sulfinamide, 7R

To a stirred solution of compound 5R (17.5 g, 58.0 mmol) in THF (70 mL) at −5° C. was added a 1 M solution of 4-F-Phenyl magnesium bromide in THF (116 mL, 116 mmol, 2 equiv.) dropwise. The resulting reaction mixture was stirred at −5° C. for 4 h followed by room temperature for 14 h. TLC (50% EtOAc/hexanes) indicated the complete conversion of the starting material. The reaction mixture was then cooled to 0° C. and saturated aqueous NH₄Cl (70 mL) was added dropwise. After warming to room temperature, the aqueous phase was extracted with MTBE and the combined organic layer was washed with water followed by drying over Na₂SO₄. Evaporation of the solvent gave the crude product which was re-slurried with heptane followed by filtration to give compound 7R (18.24 g, 79%) as a white solid. ¹H NMR (400 MHz, CDCl₃) δ 7.72-7.68 (m, 2H), 6.98-6.94 (m, 2H), 4.51 (s, 1H), 3.67-3.60 (m, 2H), 3.35 (dd, J=11.3 Hz and 2.6 Hz, 1H), 3.27 (dd, J=11.3 Hz and 2.5 Hz, 1H), 2.70-2.63 (m, 1H), 2.33-2.27 (m, 1H), 2.05-2.01 (m, 1H), 1.98-1.88 (m, 1H), 1.76-1.66 (m, 1H), 1.62-1.45 (m, 2H), 1.17 (s, 9H), 0.84 (s, 3H), 0.69 (s, 3H).

### General Procedure for the Synthesis of (R)-7-(4-fluorophenyl)-3,3-dimethyl-1,5-dioxaspiro[5.5]undecan-7-amine, 8R

To a suspension of compound 7R (45.0 g, 113 mmol) in methanol (180 mL) at 0° C. was added a solution of 3 M HCl in methanol (113 mL, 339 mmol, 3 equiv.) dropwise. The resulting reaction mixture was allowed to warm to room temperature and stirred for 12-14 h. After completion of the reaction, the mixture was cooled to 0° C. and saturated aqueous NaHCO₃ (225 mL) was added dropwise. To the resulting suspension, CH₂Cl₂ (90 mL) was added to dissolve the product and the phases were separated. The aqueous phase was extracted with CH₂Cl₂ (2×90 mL), and the combined organics were washed with brine and dried (Na₂SO₄) and concentrated to afford the crude compound 8R (27.1 g, 82% quant) as a white solid, which was carried to the next step without further purification. ¹H NMR (400 MHz, CDCl₃) δ 7.62-7.52 (m, 2H), 6.99-6.90 (m, 2H), 3.57 (dd, J=23.4, 11.4 Hz, 2H), 3.16 (ddd, J=11.2, 8.4, 2.7 Hz, 2H), 2.53-2.36 (m, 2H), 1.86-1.34 (m, 8H), 0.59 (s, 3H), 0.36 (s, 3H).

### General Procedure for the Synthesis of (R)—N-(7-(4-fluorophenyl)-3,3-dimethyl-1,5-dioxaspiro[5.5]undecan-7-yl)formamide, 9R

A reactor equipped with stirrer, thermocouple and a nitrogen inlet was charged Ac₂O (32 mL, 339.56 mmol, 3 equiv.) and HCO₂H (12.8 mL, 339.56 mmol, 3 equiv.) at room temperature. The reaction mixture was heated to 60° C. and aged for 3 h followed by cooling to 0° C. To the above mixture at 0° C. was added a solution of 8R (26.10 g, 88.96 mmol) in CH₂Cl₂ (125 mL) using an addition funnel, and the mixture was stirred at 0° C. for 1.5-2 h. The HPLC and TLC (30% EtOAc/hexanes) indicated the complete conversion of the starting material to the desired product. The reaction was then quenched with drop-wise addition of a saturated aqueous solution of NaHCO₃ (520 mL) at 0° C. using an addition funnel. The resulting solution was stirred for 30 minutes at 0° C. before being transferred to a separating funnel. The phases were separated, and the aqueous phase was extracted with CH₂Cl₂). The combined organic layer was washed with water and distilled down to a low volume MeOH was then added and distilled to remove the remaining amount of CH₂Cl₂. The resulted MeOH solution was transferred to a 3-neck round bottom flask and additional MeOH was added and heated at 55° C. for 1 h while stirring. To the above stirring solution was then added water) dropwise using an addition funnel over 1 h. The resulted off white suspension was aged at 50° C. for 1 h and allowed to cool to room temperature for 12-14 h. The suspension was then filtered, and the cake was washed with a mixture of 1:1 MeOH/water and dried under suction and then in a vacuum oven for 24 h at 40° C. to obtain compound 9R as a white solid (28.16 g, 98%). ¹H NMR (400 MHz, CDCl₃) δ 8.29 (d, J=1.9 Hz, 1H), 8.04 (d, J=12.5 Hz, 1H), 7.49-7.37 (m, 2H), 7.03-6.89 (m, 2H), 6.43 (d, J=12.5 Hz, 1H), 6.23 (s, 1H), 3.57 (ddd, J=24.9, 11.4, 7.2 Hz, 2H), 3.24-3.13 (m, 2H), 2.91 (dq, J=13.6, 3.0 Hz, 1H), 2.72-2.51 (m, 2H), 2.42-2.27 (m, 1H), 2.10-1.97 (m, 1H), 1.71-1.59 (m, 3H), 1.58-1.33 (m, 2H), 0.59 (d, 3H), 0.28 (d, J=23.3 Hz, 3H).

### General Procedure for the Synthesis of 10R

A reactor equipped with a chiller, thermocouple and an overhead stirrer was charged compound 9R (75 g, 0.23 mol) followed by THF and stirred for not less than 15 min. To this reactor was then charged NaBH₄ (26.5 g, 0.7 mmol) portion-wise. The resulted suspension was cooled to −10° C. to 0° C. A solution of iodine (71.1 g 0.28 mol) in THF was added to the above suspension drop-wise using a dropping funnel, maintaining the internal temperature between −5° C. and 10° C. (Caution: rapid gas evolution). Upon completion of the addition of iodine, the mixture was gradually warmed to 35-45° C. over 30 min and stirred at that temperature for 2-4 h. IPC by HPLC shows the conversion of 9 to 10. The resulting white suspension was cooled to −5° C. to 0° C. and MeOH was added drop-wise over 90 min keeping the internal temperature below 10° C. Upon completion of the addition, the mixture was heated to 40-45° C. over 30 min and aged at that temperature. The complete decomposition of the intermediate amine borane complex was monitored by ¹⁹F NMR studies of the reaction mixture.

In a separate flask, a solution of NaOH (aq) was prepared by dissolving 0.930 g of the NaOH in 9 V of water. A portion (20%, 18 V) of this NaOH(aq) solution was initially added drop-wise to the above reaction mixture at 40-45° C. and aged at this temperature. A formation of a white precipitate was observed immediately upon addition of the NaOH(aq) solution. The rest of the NaOH(aq) solution was then added slowly over 2.5-3 h at 40-45° C. Upon completion of the addition, the internal temperature of the suspension was set to 20-25° C. and aged at this temperature. The suspension was then filtered, and the cake was washed with a mixture of MeOH/water (1:1) and dried under suction. The cake was then recharged into the reactor followed by water The resulted suspension was heated to 55-65° C. and aged at this temperature. The suspension was then cooled to 20-25° C. and aged for 30 min before filtering. The cake was then washed with a 1:1 mixture of MeOH/water and dried under suction and then in the vacuum oven at 55-60° C. The product, compound 10R, was obtained as white solid in 71% (54.8 g) yield with 93.2 wt % by NMR, 3.2% KF and 99.4 A % purity by HPLC. ¹H NMR (400 MHz, CDCl₃) δ 7.43-7.35 (m, 2H), 7.00-6.92 (m, 2H), 3.56 (dd, J=32.3, 11.1 Hz, 2H), 3.10 (ddd, J=19.8, 11.1, 2.7 Hz, 2H), 2.51-2.39 (m, 1H), 2.28 (td, J=13.3, 3.8 Hz, 1H), 2.04 (s, 3H), 1.87-1.58 (m, 4H), 1.55-1.31 (m, 2H), 0.55 (s, 3H), 0.27 (s, 3H) ppm.

### Preparation of (R)-2-(4-fluorophenyl)-2-(methylamino)cyclohexan-1-one, 11R

A 2 L jacketed reactor equipped with a chiller, thermocouple and an overhead stirrer was charged compound 10R (50 g, 93.4 wt %, 0.152 mol) followed by HPLC water (4 V) at room temperature and the agitation was started. The resulted suspension was cooled to an internal temperature of 15° C.-25° C. Concentrated HCl (12 N, 4 equiv., 50 mL, 0.76 mol) was added to the above suspension slowly maintaining the temperature below 35° C. The mixture was heated to an internal temperature of 65-75° C. and aged at this temperature not less than 12 h. Completion of the reaction was monitored by HPLC. The mixture was then cooled to 20-30° C. and MTBE was charged and stirred for not less than 10 min. The phases were separated, and the aqueous phase (bottom) was recharged to the reactor. A solution of 3N NaOH (aq) was then charged slowly using and addition funnel, keeping the internal temperature below 40° C., to bring the pH of the mixture 12-13 (measured using a pH paper). The resulted white suspension was extracted with MTBE. The combined MTBE layers were washed with water. The MTBE layer was check by ¹H NMR for the removal of neopentyl glycol side product (not more than 10%, if >10% repeat the water washings). The MTBE phase was concentrated to 2 V. Additional MTBE was added and concentrated again and this process was repeated two times. The resulted MTBE phase was diluted with MTBE and KF was obtained (KF=not more than 0.2%).

### HCl Salt Formation of 11R

To the above MTBE solution containing the free base compound 11R at room temperature was added a solution of 5-6 N HCl in IPA (45.48 mL, 0.228 mol, 1.5 eqiuv.) drop wise keeping the internal temperature below 30° C., a formation of a white precipitate was observed. Upon completion of addition, the resulted suspension was aged at 20-30° C. for not less than 12 h. The suspension was then filtered, and the cake was washed with MTBE (3 V×3, displacement wash, cake wash and slurry wash) and dried under suction for not less than 30 min.

The cake was then dried in a vacuum oven at 35-45° C. not less than 12 h. The product 11R·HCl was obtained as white solid in 91% (34.2 g) yield with 99.9 A % HPLC purity, 100.1 wt % by HPLC and 0.152% KF. ¹H NMR (400 MHz, DMSO-d₆) δ 9.81 (d, J=302.4 Hz, 2H), 7.47-7.28 (m, 4H), 2.44 (p, J=1.9 Hz, 1H), 2.39-2.19 (m, 2H), 2.14 (td, J=13.5, 3.9 Hz, 1H), 2.06 (s, 3H), 1.96-1.85 (m, 1H), 1.85-1.72 (m, 1H), 1.66-1.38 (m, 2H).

Using HPLC, this product was determined to be greater than 99% pure and anhydrous.

### Example 2

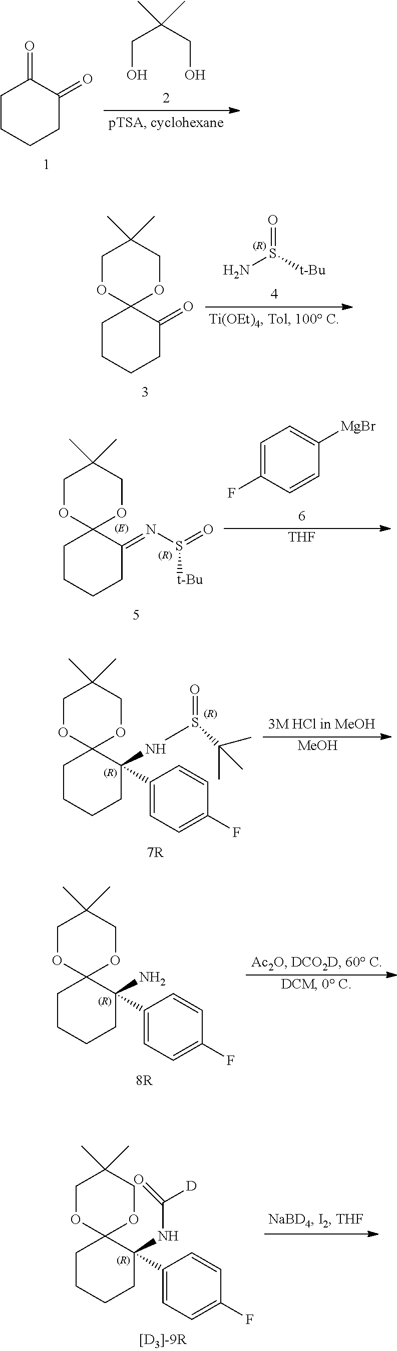

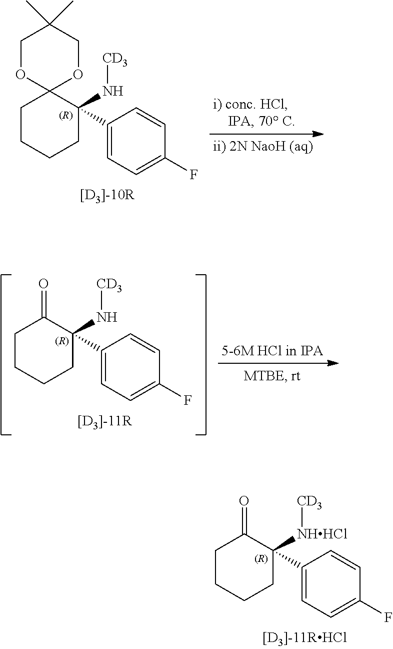

### General Procedure for the Synthesis of 3

A jacketed reactor equipped with stirrer and a Dean-Stark apparatus was charged with 1,2-cyclohexanedione (50.0 g, 428 mmol), 2,2-dimethylpropane-1,3-diol (54.0 g, 514 mmol), p-TSA (1.66 g, 8.6 mmol) and cyclohexane (200 mL, 4 V) and the resulted suspension was heated at reflux for 3 h to obtain a complete conversion of the starting material. It was then cooled to 20° C. and charged with 1N NaOH(aq) followed by MTBE and stirred. The phases were separated, and the aqueous phase was further extracted with MTBE and the combined organics were washed once with 10% brine and concentrated. The mixture was azeotroped once with toluene to obtain the 113 g (Q-NMR assay: 66%, yield 87.6%). The crude product was taken to the next step without further purification. ¹H NMR (400 MHz, CDCl₃) δ 3.55 (d, J=11.1 Hz, 1H), 3.32 (d, J=11.1 Hz, 1H), 2.38-2.35 (m, 2H), 1.8-1.79 (m, 2H), 1.67-1.63 (m, 4H), 1.06 (s, 3H), 0.55 (s, 3H).

### General Procedure for the Synthesis of 5

A 1 L round bottom flask equipped with stirrer was charged compound 3 (33.3 g, 60% wt. %, 0.101 mol), (R)-t-Bu-Sulfinamide (14.62 g, 0.121 mol), toluene (80 mL) and Ti(OEt₄) (25.31 mL, 0.121 mol) at room temperature. The mixture was heated at 80 C for 5-6 h followed by cooling to room temperature to obtain a dark solution. To this solution was added EDTE (47.5 g, 2 equiv.) and the mixture was heated at 55 C for 60 minutes followed by cooling to room temperature. To the above solution was added 12% NaCl (aq) stirred for about 5 mins and allowed to settle. The phases were separated, and the aqueous phase was re-extracted with toluene The combined organics were washed with water. The organic phase was filtered through a plug of activated charcoal and SiO₂ and concentrated to obtain the crude product as yellow-orange semi solid (18.9 g net product by NMR wt %, 63%). The product crystalized out as off-white solid upon standing, which was filtered and carried to the next step. ¹H NMR (400 MHz, CDCl₃) δ 3.83 (d, J=11.0 Hz, 1H), 3.72 (d, J=11.0 Hz, 1H), 3.44-3.38 (m, 2H), 3.13-3.07 (m, 1H), 2.89-2.83 (m, 1H), 1.98-1.85 (m, 1H), 1.81-1.71 (m, 1H), 1.31 (s, 9H), 1.21 (s, 3H), 0.72 (s, 3H).

### General Procedure for the Synthesis of 7R

To a stirred solution of compound 6 (17.5 g, 58.0 mmol) in THF (70 mL) at −5° C. was added a 1 M solution of 4-F-Phenyl magnesium bromide in THE (116 mL, 116 mmol, 2 equiv.) dropwise. The resulting reaction mixture was stirred at −5° C. for 4 h followed by room temperature for 14 h. TLC (50% EtOAc/hexanes) indicated the complete conversion of the starting material. The reaction mixture was then cooled to 0° C. and saturated aqueous NH₄Cl (70 mL) was added dropwise. After warming to the room temperature, the aqueous phase was extracted with MTBE and the combined organic layer was washed with water and then dried over Na₂SO₄. Evaporation of the solvent gave the crude product which was re-slurried with heptane followed by filtration to give compound 7 (18.24 g, 79%) as a white solid. ¹H NMR (400 MHz, CDCl₃) δ 7.72-7.68 (m, 2H), 6.98-6.94 (m, 2H), 4.51 (s, 1H), 3.67-3.60 (m, 2H), 3.35 (dd, J=11.3 Hz and 2.6 Hz, 1H), 3.27 (dd, J=11.3 Hz and 2.5 Hz, 1H), 2.70-2.63 (m, 1H), 2.33-2.27 (m, 1H), 2.05-2.01 (m, 1H), 1.98-1.88 (m, 1H), 1.76-1.66 (m, 1H), 1.62-1.45 (m, 2H), 1.17 (s, 9H), 0.84 (s, 3H), 0.69 (s, 3H).

### General Procedure for the Synthesis of 8R

To a suspension of compound 7 (45.0 g, 113 mmol) in methanol (180 mL) at 0° C. was added a solution of 3 M HCl in methanol (113 mL, 339 mmol, 3 equiv.) dropwise. The resulting reaction mixture was allowed to warm to room temperature and stirred for 12-14 h. After completion of the reaction, the mixture was cooled to 0° C. and saturated aqueous NaHCO₃ (225 mL) was added dropwise. To the resulting suspension, CH₂Cl₂ was added to dissolve the product and the phases were separated. The aqueous phase was extracted with CH₂Cl₂, and the combined organics were washed with brine and dried (Na₂SO₄) and concentrated to afford the crude compound 8 (27.1 g, 82% quant) as a white solid, which was carried to the next step without further purification. ¹H NMR (400 MHz, CDCl₃) δ 7.62-7.52 (m, 2H), 6.99-6.90 (m, 2H), 3.57 (dd, J=23.4, 11.4 Hz, 2H), 3.16 (ddd, J=11.2, 8.4, 2.7 Hz, 2H), 2.53-2.36 (m, 2H), 1.86-1.34 (m, 8H), 0.59 (s, 3H), 0.36 (s, 3H).

### General Procedure for the Synthesis of [D]-9R

A mixture of acetic anhydride (1.9 mL, 13.63 mmol) and formic acid-d₃ (0.54 mL, 13.63 mmol) was stirred at 60 C for 2 h followed by gradually cooling to 0 C. The above mixture at 0 C was then added a solution of compound 8 (1.0 g, 3.41 mmol) in CH₂Cl₂ (5 mL) and the mixture was allowed to stir at 0 C for 2 h. TLC (30% EtOAc/hexanes) indicated the complete conversion of the starting material. The mixture was then neutralized by slow addition of an aqueous solution of sodium bicarbonate and extracted with CH₂Cl₂. The combined organics were washed once with satd. NaHCO₃ (aq), water followed by brine, dried (Na₂SO₄) and concentrated to obtain the crude [D]-9 (1.1 g, quantitative) as off-white solid, which was carried to the next step without further purification. ¹H NMR (400 MHz, CDCl₃) δ 7.50-7.36 (m, 2H), 7.02-6.88 (m, 2H), 6.56-6.11 (m, 1H), 3.66-3.49 (m, 2H), 3.26-3.11 (m, 2H), 2.98-2.87 (m, 1H), 2.71-2.52 (m, 2H), 2.42-2.29 (m, 1H), 2.11-2.00 (m, 1H), 1.71-1.32 (m, 4H), 0.62-0.57 (m, 3H), 0.33-0.23 (m, 3H).

### General Procedure for the Synthesis of [D3]-10R

To a stirring suspension of [D]-9 (1.1 g, 3.42 mmol) and NaBD₄ (572 mg, 13.66 mmol) in THF (4 mL) at 0 C was added a solution of Iodine (1.13 g, 4.44 mmol) in THE (2 mL) drop-wise. The mixture was then allowed to warm to room temperature for 14 h. The mixture was then cooled to 0 C and quenched with slow addition of MeOH (2 mL) followed by heating at 40 C for 1 h. The resulted clear solution was then concentrated and treated with MTBE followed by water and 1N NaOH(aq) to obtain clear phase separation. The MTBE later was separated, and the aqueous phase was further extracted with MTBE. The combined organics were then washed with water followed by brine, dried (Na₂SO₄) and concentrated. The crude mixture was purified by chromatography on SiO₂ (100% hexane to 30-50% EtOAc/hexanes) to obtain [D3]-10 (710 mg, 67%) as white solid. ¹H NMR (400 MHz, CDCl₃) δ 7.45-7.32 (m, 2H), 7.02-6.90 (m, 2H), 3.56 (dd, J=32.3, 11.1 Hz, 2H), 3.16-3.03 (m, 2H), 2.51-2.41 (m, 1H), 2.27 (td, J=13.3, 3.8 Hz, 1H), 1.86-1.57 (m, 4H), 1.55-1.31 (m, 2H), 0.55 (s, 3H), 0.26 (s, 3H); ¹⁹F NMR (376 MHz, CDCl₃) δ −118.7.

### General Procedure for the Synthesis of [D3]-11R Free Base

To a solution of [D3]-10 (640 mg, 2.6 mmol) in IPA (4 V) at room temperature was added a solution of conc. HCL (4 equiv.) and the mixture was heated at 70 C for 14 h to obtain a complete conversion of the starting material. The mixture was then basified with a solution of 3N NaOH (aq) and extracted with MTBE. The combined organics were washed once with water, dried (Na₂SO₄) and concentrated to obtain crude [D3]-11 (430 mg, 93%) as colorless oil, which was carried to the next step without further purification.

### General Procedure for the Synthesis of [D3]-11R—HCl Salt

To a solution of crude [D3]-11 free base (430 mg) in MTBE (5 mL) was added a solution of HCl in IPA (1.5 equiv.) drop-wise at room temperature. A formation of a white suspension was observed during the addition of the HCl solution. The resulted white suspension was then allowed to stir at room temperature for 12-14. It was then filtered and washed with MTBE (3×3 V) to obtain the [D3]-11-HCl salt (420 mg, 84%) as a white solid. ¹H NMR (400 MHz, DMSO) δ 9.82 (s, 1H), 9.34 (s, 1H), 7.53-7.32 (m, 4H), 3.15 (dt, J=13.8, 3.0 Hz, 1H), 2.45-2.27 (m, 2H), 2.16-2.03 (m, 1H), 2.02-1.79 (m, 2H), 1.72-1.48 (m, 2H).

### Example 3

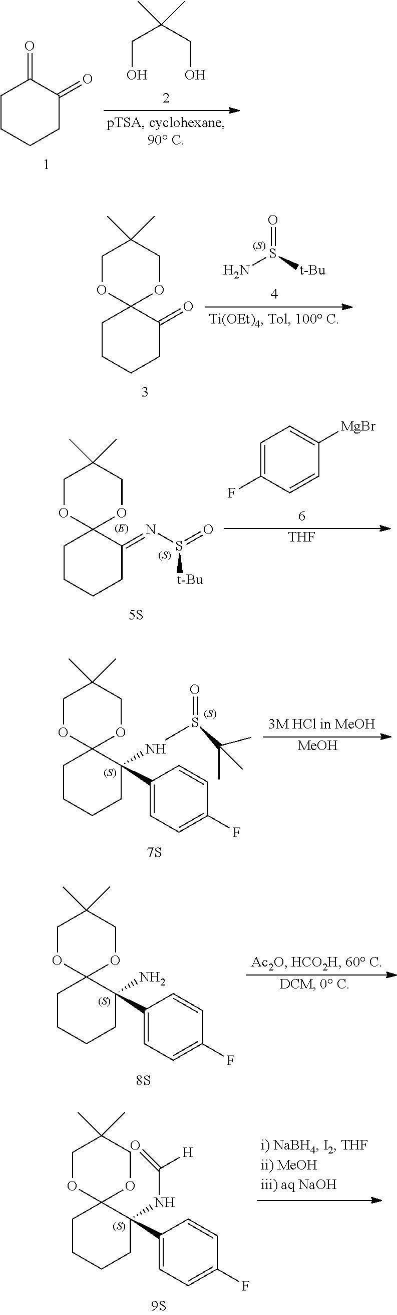

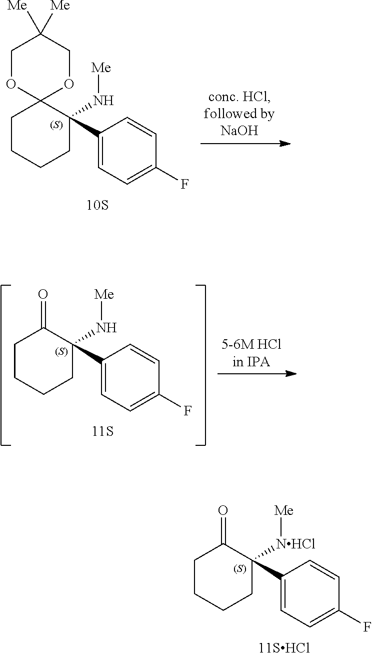

Using the procedures of Example 1 and substituting (S)-t-Bu-sulfinamide for (R)-t-Bu-sulfinamide, the compounds identified in the scheme hereinabove are prepared.

### Example 4

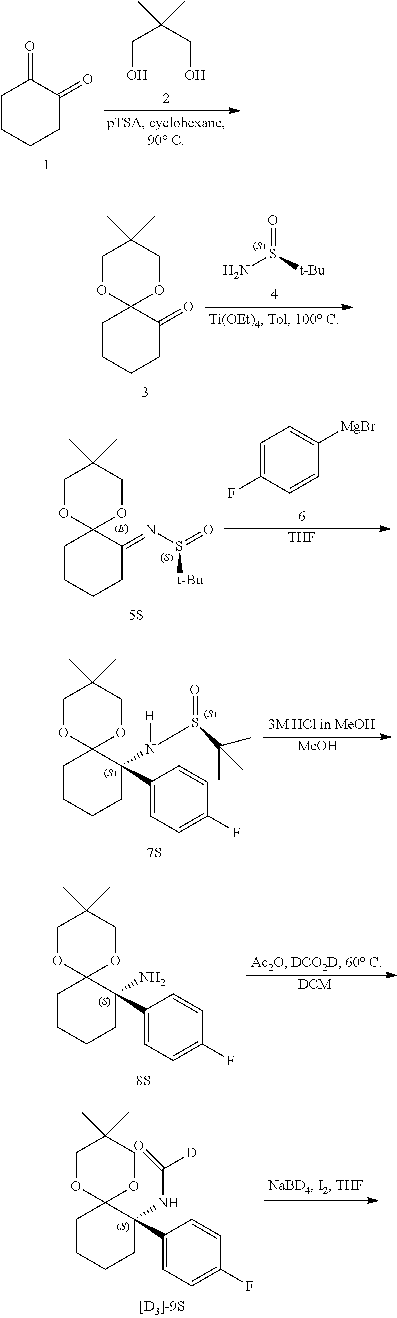

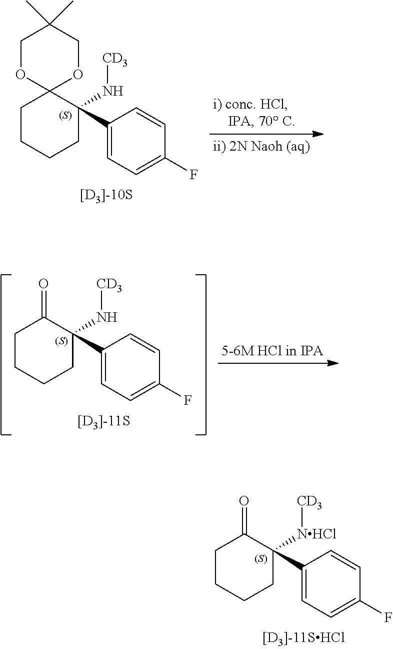

Using the procedures of Example 2 and substituting (S)-t-Bu-sulfinamide for (R)-t-Bu-sulfinamide, the compounds identified in the scheme hereinabove are prepared.

While certain features of the invention have been illustrated and described herein, many modifications, substitutions, changes, and equivalents may occur to those of ordinary skill in the art. It is, therefore, to be understood that the appended claims are intended to cover all such modifications and changes as fall within the true spirit of the invention.

1. A compound of the formula: 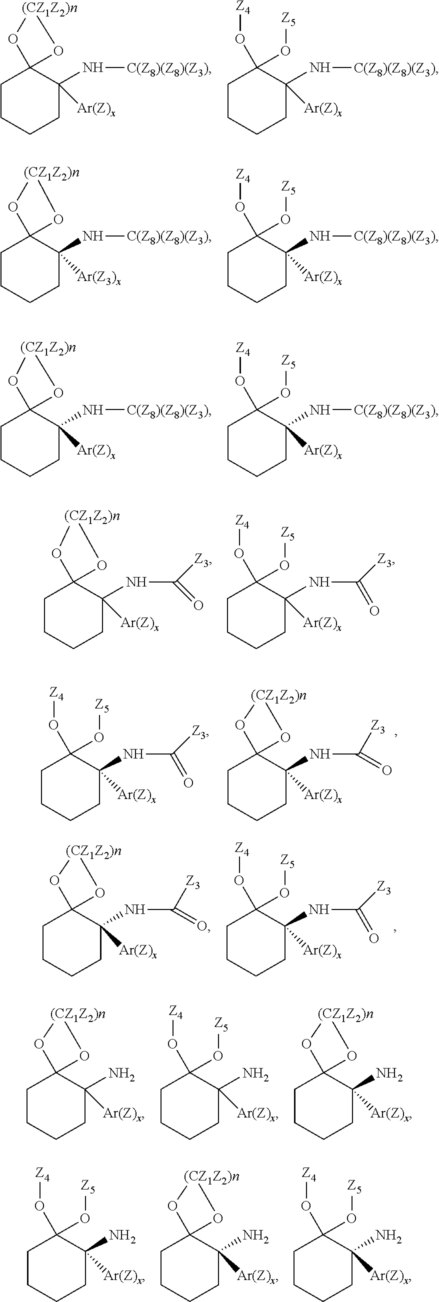 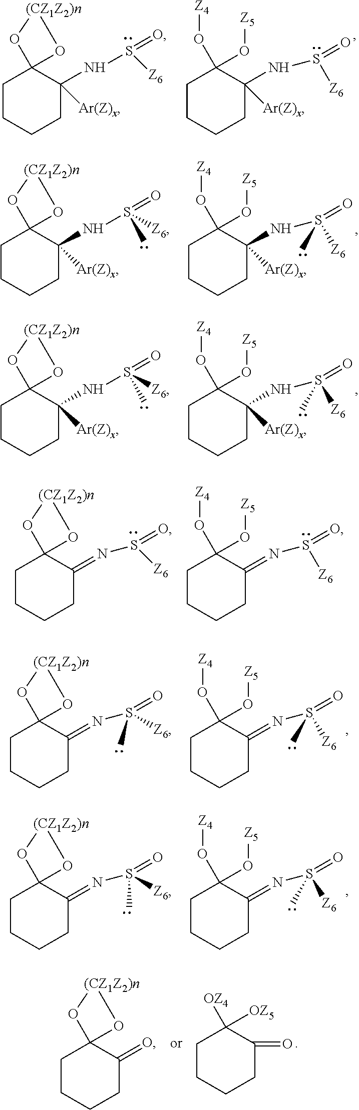
wherein
each Z₁ is independently H or C1-C6 alkyl;
each Z₂ is independently H or C1-C6 alkyl;
n is 2 or 3;
Z₃ is H, D, C1-C6 alkyl optionally substituted with one or more halogen or C1-C6 alkoxy, or aryl optionally substituted with one or more C1-C6 alkyl, C1-C6 alkoxy, or halogen;
Z₄ is C1-C6 alkyl;
Z₅ is C1-C6 alkyl;
Z₆ is C1-C6 alkyl, C3-C10 cycloalkyl, hydrocarbyl aryl, or hydrocarbyl Ar(C1-C3) alkyl, each of which may be unsubstituted or substituted with one or more halo, C1-C6 alkyl or C1-C6 alkoxy;
each Z₈ is independently H or D;
each Z is independently halogen (selected from F, Cl, Br, I), —OZ₁₁, C1-C10 alkyl, C2-C10 alkenyl, C3-C10 alkynyl having no terminal hydrogen atoms, CF₃, or OCF₃; wherein each Z₁ is independently selected from C1-C10 alkyl, C2-C10 alkenyl, C3-C10 alkynyl having no terminal hydrogen atoms, and aryl which is unsubstituted or substituted with C1-C6 alkyl, C1-C6 alkoxy, or halogen;
Ar is aryl;
each Z₈ is independently H or D; and
x is 0, 1, 2, 3, 4 or 5;
with the proviso that when Ar is thiophene, x is 0, 1, 2, or 3,
and with the further provisos that the following compounds are excluded:

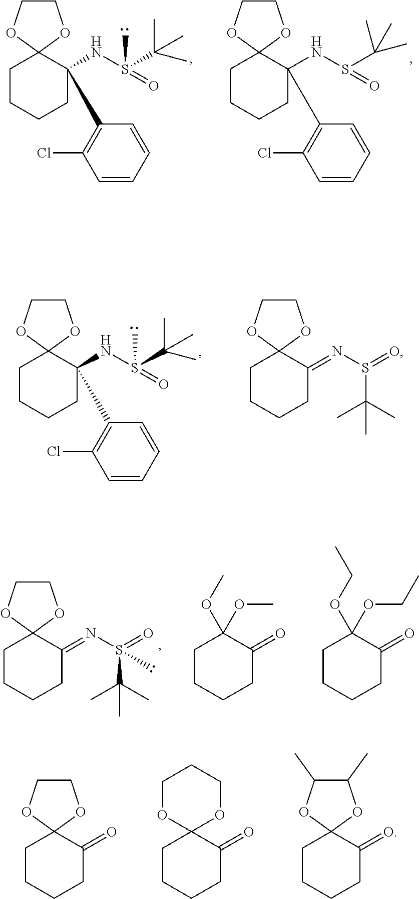

2. The compound according to claim 1 wherein Z₆ is C1-C6 alkyl, hydrocarbyl aryl, or C5-C10 cycloalkyl.

3. The compound according to claim 1 wherein Z₆ is t-butyl, phenyl, tolyl, or adamantyl.

4. The compound according to claim 1 wherein each Z₁ and Z₂ are independently H or C1-C3 alkyl and Z₄ and Z₅ are independently C1-C3 alkyl.

5. The compound according to claim 1 wherein each pair of Z₁ and Z₂ are the same and each pair of Z₃ and Z₄ are the same.

6. The compound according to claim 1 wherein Z₃ is H, D, or C1-C6 alkyl optionally substituted with one or more fluoro or C1-C3 alkoxy.

7. The compound according to claim 1 wherein Z₃ is H or D.

8. The compound according to claim 1 wherein Z₃ is D when each Z₈ is D.

9. The compound according to claim 1 wherein Ar is phenyl.

10. The compound according to claim 1 wherein x is 0, 1, or 2.

11. The compound according to claim 1 wherein x is 1 or 2 and each Z is independently halogen, —OZ₁₁, or C1-C10 alkyl.

12. The compound according to claim 1 wherein at least one Z is F.

13. The compound according to claim 1 wherein Ar is phenyl and at least one Z is F at the 4-position of Ar.

14. The compound according to claim 1 wherein x is 1 and Z is F.

15. A compound of the formula:

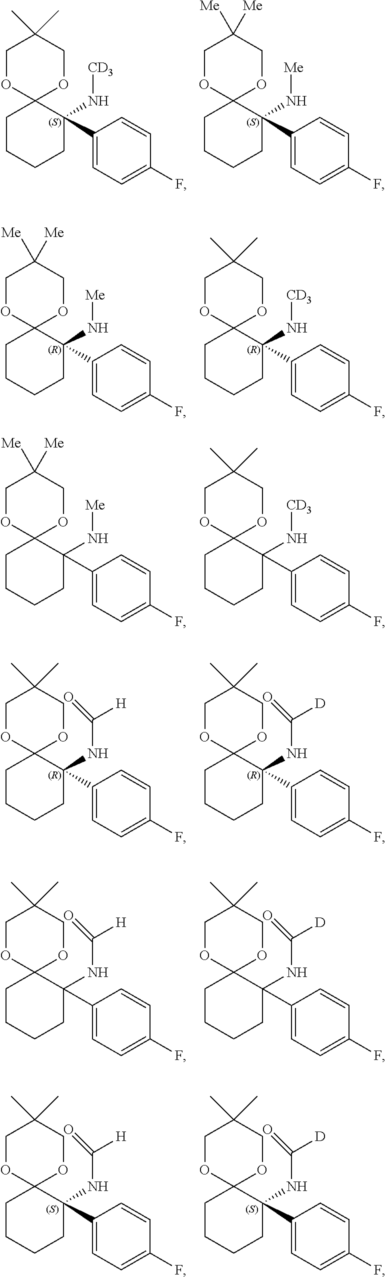

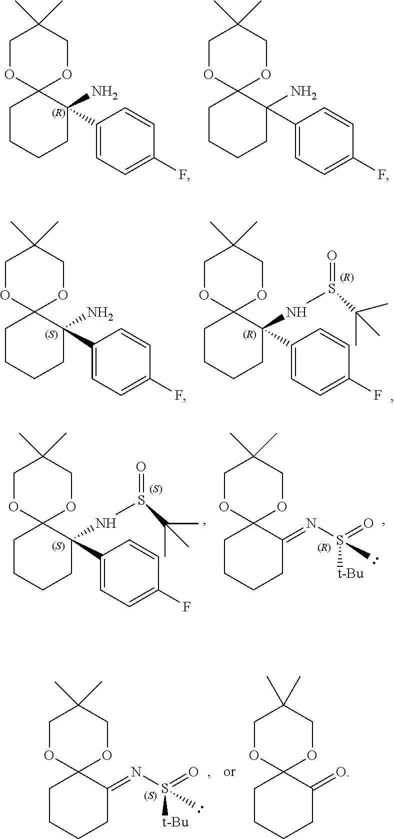

16-55. (canceled)

56. A process for preparing a compound of Formula I 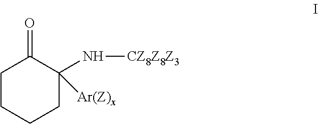
or the R isomer thereof of the formula 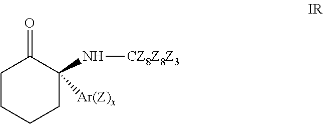
or the S isomer thereof of the formula 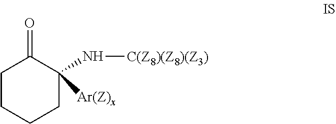
comprising
(i) with respect to the compound of formula I:
(1) (a)reacting a compound having a ketal protecting group of the formula 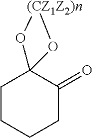
with a sulfinamide of the formula 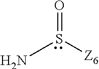
in the presence of Ti(OZ₇)a(X₁)_{b} to form an imine of Formula V 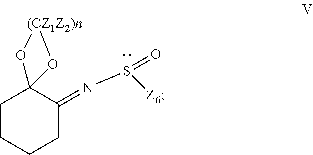
(b) reacting the imine of Formula V with an aryl nucleophilic agent Nu, capable of delivering an aryl nucleophilic moiety of the formula Ar(Z)ₓ under arylation reaction conditions to form a sulfinamide of Formula VII 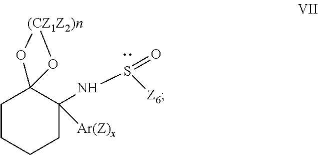
(c) reacting the sulfinamide of Formula VII with acid to form the corresponding amine of Formula VIII 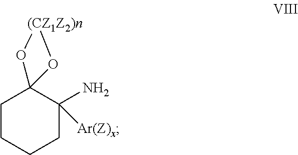
(d) reacting the amine of Formula VIII with an acylating agent of formula Z₃COOH or acid derivative thereof or when Z₃ is D, with Z₃COOXₒ or a deuterated acylating agent thereof under amide forming conditions to form an amide of Formula IX having an acyl group C(═O)Z₃ 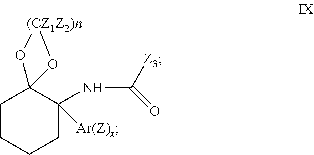
(e) reducing the acyl group in Formula IX with an acyl reducing agent under acyl reducing conditions or when Z₈ is D, with a deuterated acyl reducing agent under acyl reducing conditions to form a compound of Formula X 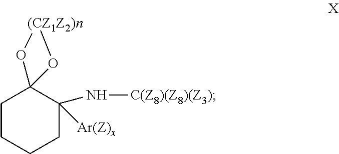
and
(f) deprotecting the ketal of the compound of Formula X to form a compound of formula I; or
(2)(a) reacting a compound having a ketal protecting group of the formula 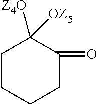
with a sulfinamide of the formula 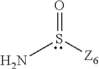
in the presence of Ti(OZ₇)ₐ(X₁)_{b} to form an imine of Formula VA 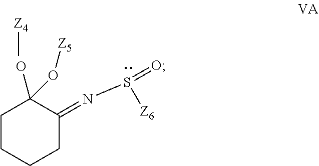
(b) reacting the imine of Formula VA with an aryl nucleophilic agent Nu, capable of delivering an aryl nucleophilic moiety of the formula Ar(Z)ₓ under arylation reaction conditions to form a sulfinamide of Formula VIIA 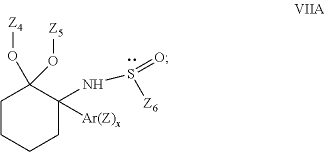
(c) reacting the sulfinamide of Formula VIIA with acid to form the corresponding amine of Formula VIIIA 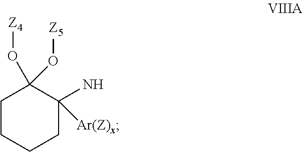
(d) reacting the amine of Formula VIIIA with an acylating agent of formula Z₃COOH or acid derivative thereof or when Z₃ is D, with Z₃COOXₒ or a deuterated acylating agent thereof to form an amide of Formula IXA having an acyl group C(═O)Z₃ 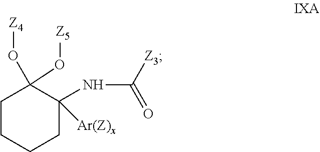
(e) reducing the acyl group in Formula IXA with an acyl reducing agent or when Z₈ is D, with a deuterated acyl reducing agent under acyl reducing conditions to form a compound of Formula XA 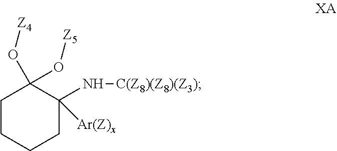
and
(f) deprotecting the ketal of Formula XA to form a compound of Formula I;
(ii), with respect a compound of formula IR:
(a) reacting a compound having a ketal protecting group of formula 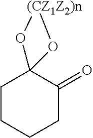
with a sulfinamide of the formula 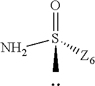
in the presence of Ti(OZ₇)a(X₁)_{b} to form an imine of Formula VR 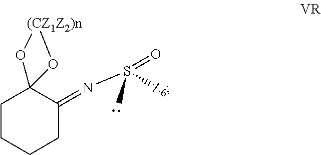
(b) reacting the imine of Formula VR with an aryl nucleophilic agent Nu, capable of delivering an aryl nucleophilic moiety of the formula Ar(Z)ₓ under arylation reaction conditions to form a sulfinamide of Formula VIIR 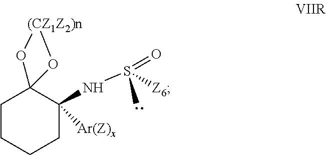
(c) reacting the sulfinamide of Formula VIIR with acid to form the corresponding amine of Formula VIIIR 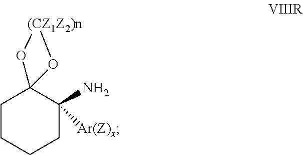
(d) reacting the amine of Formula VIIIR with an acylating agent of formula Z₃COOH or acid derivative thereof or when Z₃ is D, with Z₃COOX₀ or deuterated acylating agent thereof under amide forming conditions to form an amide of Formula IXR having an acyl group C(═O)Z₃ 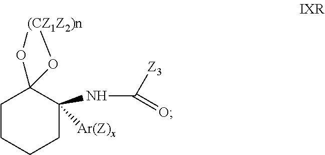
(e) reducing the acyl group in Formula IXR with an acyl reducing agent under acyl reducing conditions or when Z₈ is D, with a deuterated acyl reducing agent under acyl reducing conditions to form a compound of Formula XR 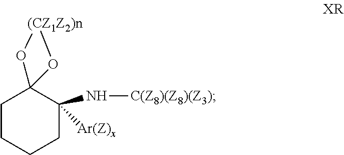
and
(f) deprotecting the ketal of Formula XR to form a compound of Formula IR; or
(2)(a) reacting a compound having a ketal protecting group of the formula 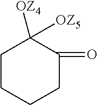
with a sulfinamide of the formula 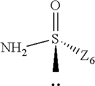
in the presence of Ti(OZ₇)ₐ(X₁)_{b} to form an imine of Formula VAR 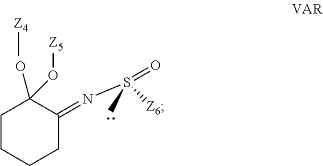
(b) reacting the imine of Formula VAR with an aryl nucleophilic agent Nu, capable of delivering an aryl nucleophilic moiety of the formula Ar(Z)ₓ under arylation reaction conditions to form a sulfinamide of Formula VIIAR 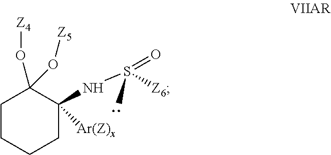
(c) reacting the sulfinamide of Formula VIIAR with acid to form the corresponding amine of Formula VIIIAR 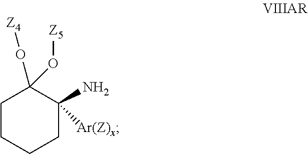
(d) reacting the amine of Formula VIIIAR with an acylating agent of Formula Z₃COOH or acid derivative thereof or when Z₃ is D, with Z₃COOXₒ or deuterated acylating agent thereof to form an amide of Formula IXAR having an acyl group C(═O)Z₃ 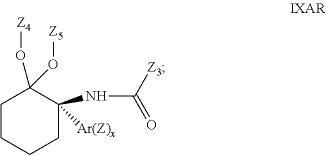
(e) reducing the acyl group in Formula IXAR with an acyl reducing agent or when Z₈ is D, with a deuterated acyl reducing agent under acyl reducing conditions to form a compound of Formula XAR 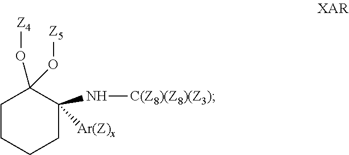
and
(f) deprotecting the ketal of Formula XAR to form a compound of Formula IAR;
(iii) with respect to a compound of formula IS:
(1) (a) reacting a compound having a ketal protecting group of the formula 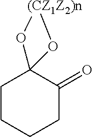
with a sulfinamide of the formula 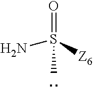
in the presence of Ti(OZ₇)ₐ(X₁)_{b} to form an imine of Formula VS 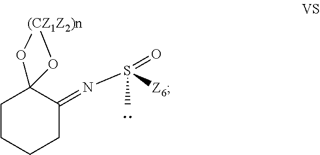
(b) reacting the imine of Formula VS with an aryl nucleophilic agent Nu, capable of delivering an aryl nucleophilic moiety of the formula Ar(Z)ₓ under arylation reaction conditions to form a sulfinamide of Formula VIIS 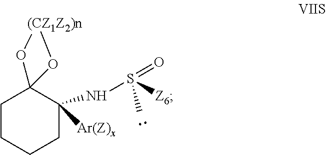
(c) reacting the sulfinamide of Formula VIIS with acid to form the corresponding amine of Formula VIIIS 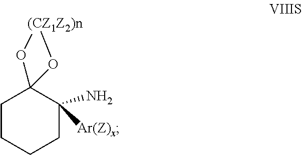
(d) reacting the amine of Formula VIIIS with an acylating agent of Formula Z₃COOH or acid derivative thereof or when Z₃ is D, with Z₃COOXₒ or deuterated acylating agent thereof under amide forming conditions to form an amide of Formula IXS having an acyl group C(═O)Z₃ 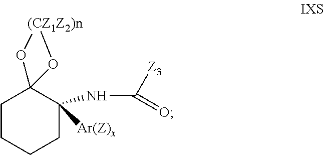
(e) reducing the acyl group in Formula IXS with an acyl reducing agent under acyl reducing conditions to form a compound of Formula XS 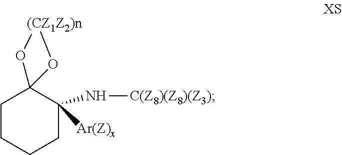
and
(f) deprotecting the ketal of Formula XS to form a compound of Formula IS; or
(2) (a) reacting a compound having a ketal protecting group of the formula 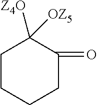
with a sulfinamide of the formula 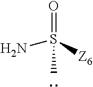
in the presence of Ti(OZ₇)ₐ(X₁)_{b} to form an imine of Formula VAS 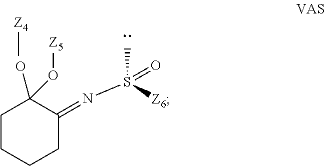
(b) reacting the imine of Formula VAS with an aryl nucleophilic agent Nu, capable of delivering an aryl nucleophilic moiety of the formula Ar(Z)ₓ under arylation reaction conditions to form a sulfinamide of Formula VIIAS 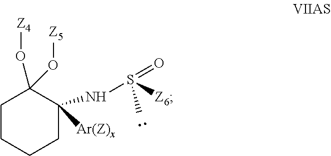
(c) reacting the sulfinamide of Formula VIIAS with acid to form the corresponding amine of Formula VIIIAS 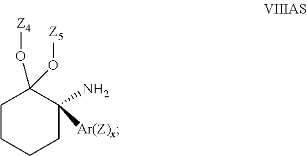
(d) reacting the amine of Formula VIIIAS with an acylating agent of Formula Z₃COOH or acid derivative thereof or when Z₃ is D, with Z₃COOXₒ or deuterated acylating agent thereof to form an amide of Formula IXAS having an acyl group C(═O)Z₃ 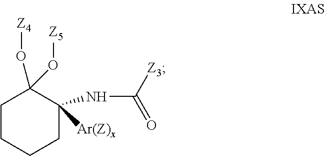
(e) reducing the acyl group in Formula IXAS with an acyl reducing agent or when Z₈ is D, with a deuterated acyl reducing agent under acyl reducing conditions to form a compound of Formula XAS 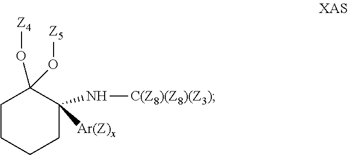
and
(f) deprotecting the ketal of Formula XAS to form a compound of Formula IS;
wherein
each Z₁ is independently H or C1-C6 alkyl;
each Z₂ is independently H or C1-C6 alkyl;
Z₄ is C1-C6 alkyl;
Z₅ is C1-C6 alkyl;
n is 2 or 3;
Z₃ is H or D, C1-C6 alkyl optionally substituted with one or more halogen or C1-C6 alkoxy, or aryl optionally substituted with one or more C1-C6 alkyl, C1-C6 alkoxy, or halogen;
Each Z is independently halogen (selected from F, Cl, Br, I), —OZ₁₁, C1-C10 alkyl, C2-C10 alkenyl, C3-C10 alkynyl having no terminal hydrogen atoms, CF₃, or OCF₃; wherein each Z₁ is independently selected from C1-C10 alkyl, C2-C10 alkenyl, C3-C10 alkynyl having no terminal hydrogen atoms, and aryl which is unsubstituted or substituted with C1-C6 alkyl, C1-C6 alkoxy, or halogen;
Z₇ is C1-C4 alkyl;
Each Z₈ is H or D;
Z₆ is C1-C6 alkyl, C3-C10 cycloalkyl, hydrocarbyl aryl, or hydrocarbyl Ar(C1-C3) alkyl, each of which may be unsubstituted or substituted with one or more halo, C1-C6 alkoxy or C1-C6 alkyl;
X₀ is H or D;
X₁ is halo;
a is 0, 1, 2, 3, or 4;
b is 0, 1, 2, 3, or 4;
a+b=4;
x is 0, 1, 2, 3, 4, or 5; and
Ar is aryl;
provided that when Ar is thiophene, x is 0, 1, 2, or 3.

57. The process according to claim 56 wherein Z₆ is C1-C6 alkyl, hydrocarbyl aryl or C5-C10 cycloalkyl.

58. The process according to claim 56 wherein Z₁ and Z₂ are independently H or C1-C3 alkyl and Z₄ and Z₅ are independently C1-C3 alkyl.

59. The process according to claim 56 wherein each pair of Z₁ and Z₂ are the same and each pair of Z₄ and Z₅ are the same.

60. The process according to claim 56 wherein Z₃ is H or C1-C6 alkyl optionally substituted with one or more fluoro or C1-C3 alkoxy.

61. The process according to claim 56 wherein Ar is phenyl.

62. The process according to claim 56 wherein x is 0, 1, or 2.

63. The process according to claim 56 wherein x is 1 or 2 and each Z is independently halogen, —OZ₁₁, or C1-C10 alkyl.

64. The process according to claim 56 wherein at least one Z is F.

65. The process according to claim 56 wherein Ar is phenyl and at least one Z is F at the 4-position of Ar.

66. The process according to claim 56 wherein x is 1 and Z is F.

67. The process according to claim 56 wherein Ti(OZ₇)ₐ(X₁)_{b} is Ti(OEt)₄, Ti(OiPr)₄, TiCl(OiPr)₃, or TiCl₂(OiPr)₂.

68. The process according to claim 56 wherein Nu is Ar(Z)ₓMgX or Ar(Z)ₓLi, wherein X is halo.

69. The process according to claim 56 wherein Nu is Ar(Z)ₓMgX, wherein X is halo.

70. The process according to claim 56 wherein the amine is formed by reacting the sulfinamide in a polar protic solvent with nitric acid, sulfuric acid, trifluoroacetic acid, para-toluene sulfonic acid, or HX₃, wherein X₃ is a halide.

71. The process according to claim 56 wherein the deprotecting occurs in the presence of a protic acid.

72. The process according to claim 56 wherein Z₃ is H or D.

73. The process according to claim 72 wherein Z₃ is D.

74. The process according to claim 72 wherein Z₃ is H.

75. The process according to claim 56 wherein 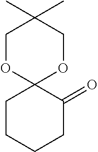
is the protected ketal and is prepared by reacting 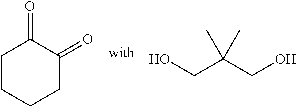
in the presence of acid.

76. A process according to claim 56 wherein the compound prepared has the formula: 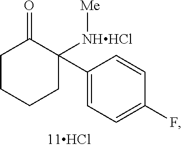
which process comprises
(a) reacting a compound having a ketal protecting group of the structure 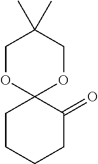
with a sulfinamide of the structure 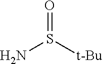
in the presence of Ti(OEt)₄ to form an imine 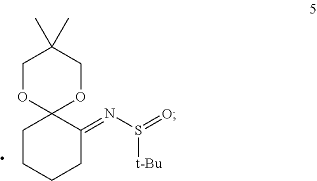 [SMILES: CC1(C)COC2(CCCC/C2=N\S(=O)C(C)(C)C)OC1]
(b) reacting the imine 5 with an organometallic compound of the structure 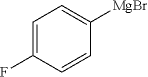
under Grignard coupling conditions to form a sulfinamide 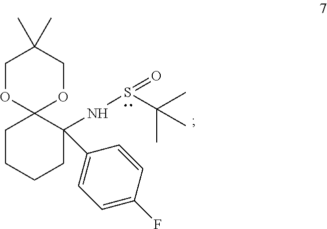 [SMILES: CC1(C)COC2(CCCCC2(NS(=O)C(C)(C)C)c2ccc(F)cc2)OC1]
(c) reacting the sulfinamide 7 with acid to form the corresponding amine 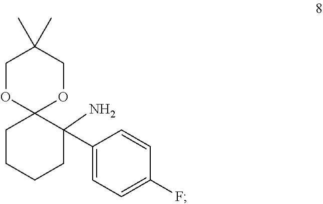 [SMILES: CC1(C)COC2(CCCCC2(N)c2ccc(F)cc2)OC1]
(d) reacting the amine 8 with an acylating agent of formula HCOOH or acid derivative thereof under amide forming conditions to form the amide 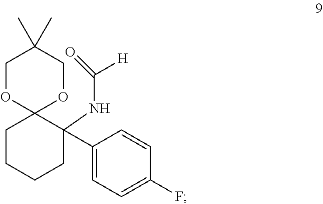 [SMILES: CC1(C)COC2(CCCCC2(NC=O)c2ccc(F)cc2)OC1]
(e) reducing the acyl group in 9 with an acyl reducing agent to form the compound 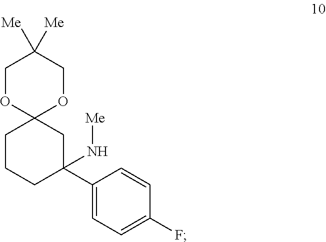 [SMILES: CNC1(c2ccc(F)cc2)CCCC2(C1)OCC(C)(C)CO2]
and
(f) deprotecting the ketal of 10 to form the compound 11 and then acidifying 11 with HCl to form compound 11·HCl.

77. The process according to claim 56 wherein the compound prepared has the formula: 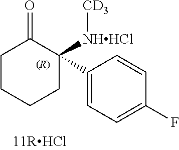
which process comprises
(a) reacting a compound having a ketal protecting group of the structure 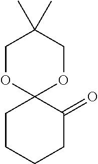
with a sulfinamide of the structure 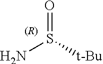
in the presence of Ti(OEt)₄ to form an imine 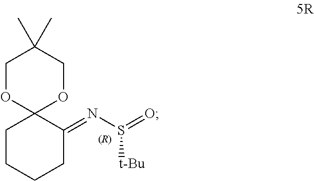
(b) reacting the imine 5R with an organometallic compound of the structure 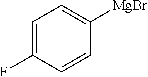
under Grignard coupling conditions to form a sulfinamide 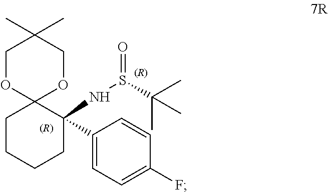
(c) reacting the sulfinamide 7R with acid to form the corresponding amine 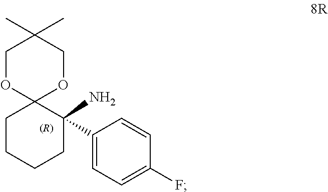
(d) reacting the amine 8R with an acylating agent of formula HCOOH or acid derivative thereof under amide forming conditions to form the amide 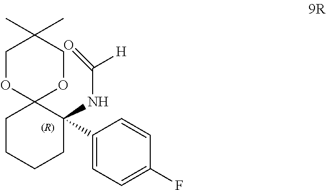
(e) reducing the acyl group in 9R with an acyl reducing agent to form the compound 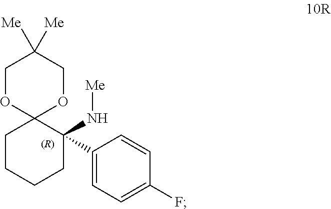
and
(f) deprotecting the ketal of 10R to form the compound 11R and then acidifying 11R with HCl to form compound 11R·HCl.

78. The process according to claim 56 wherein the compound formed has the formula: 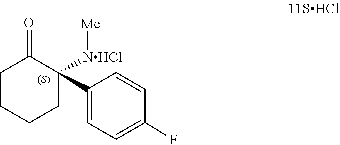
which process comprises
(a) reacting a compound having a ketal protecting group of the structure 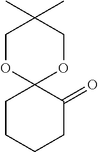
with a sulfinamide of the structure 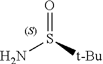
in the presence of Ti(OEt)₄ to form an imine 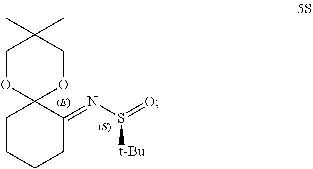
(b) reacting the imine 5S with an organometallic compound of the structure 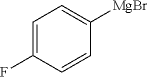
under Grignard coupling conditions to form a sulfinamide 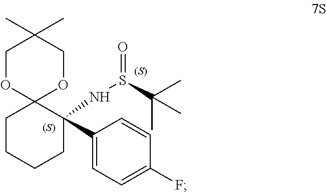
(c) reacting the sulfinamide 7S with acid to form the corresponding amine 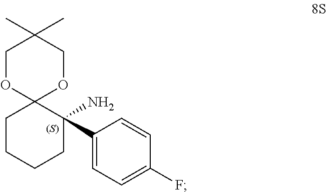
(d) reacting the amine 8S with an acylating agent of formula HCOOH or acid derivative thereof under amide forming conditions to form the amide 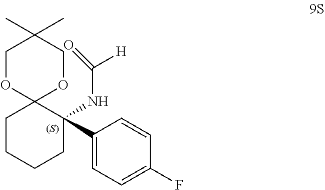
(e) reducing the acyl group in 9S with an acyl reducing agent under acyl reducing conditions to form the compound 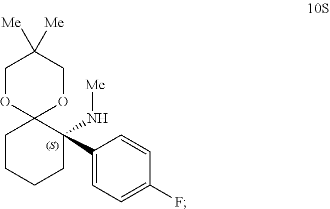
and
(f) deprotecting the ketal of 10S to form the compound 11S and then acidifying 11S with HCl to form compound 11·HCl.

79. A process according to claim 56 for preparing the compound of the formula: 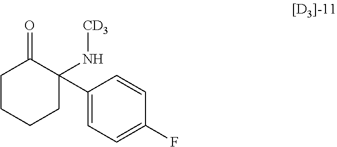 [SMILES: [2H]C([2H])([2H])NC1(c2ccc(F)cc2)CCCCC1=O]
which process comprises
(a) reacting a compound having a ketal protecting group of the structure 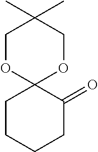
with a sulfinamide of the structure 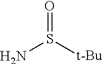
in the presence of Ti(OEt)₄ to form an imine 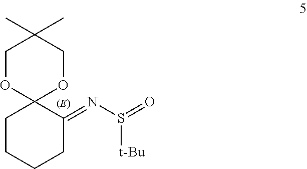 [SMILES: CC1(C)COC2(CCCC/C2=N\S(=O)C(C)(C)C)OC1]
(b) reacting the imine 5 with an organometallic compound of the structure 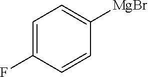
under Grignard coupling conditions to form a sulfinamide 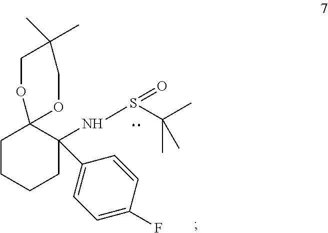 [SMILES: CC1(C)COC2(CCCCC2(NS(=O)C(C)(C)C)c2ccc(F)cc2)OC1]
(c) reacting the sulfinamide 7 with acid to form the corresponding amine 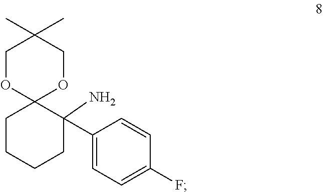 [SMILES: CC1(C)COC2(CCCCC2(N)c2ccc(F)cc2)OC1]
(d) reacting the amine 8 with a deuterated acylating agent of formula DCOOD or deuterated acid derivative thereof under amide forming conditions to form the amide 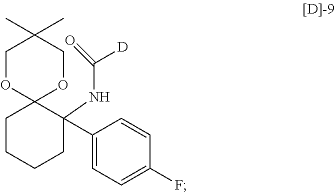 [SMILES: [2H]C(=O)NC1(c2ccc(F)cc2)CCCCC12OCC(C)(C)CO2]
(e) reducing the acyl group in [D]-9 with a deuterated acyl reducing agent under acyl reducing conditions to form the compound 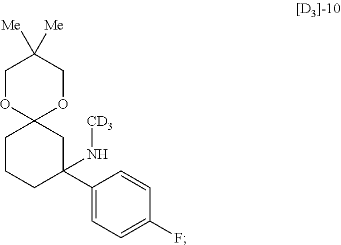 [SMILES: [2H]C([2H])([2H])NC1(c2ccc(F)cc2)CCCC2(C1)OCC(C)(C)CO2]
and
(f) deprotecting the ketal in [D₃]-10 in the presence of acid, then treating the resulting product with base to form the compound [D₃]-11.

80. A process according to claim 56 for preparing the compound of the formula: 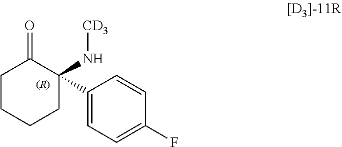
which process comprises
(a) reacting a compound having a ketal protecting group of the structure 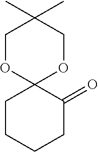
with a sulfinamide of the structure 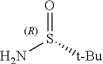
in the presence of Ti(OEt)₄ to form an imine 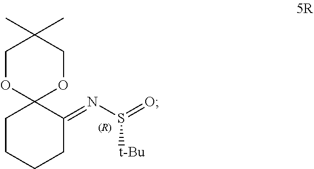
(b) reacting the imine 5R with an organometallic compound of the structure 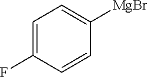
under Grignard coupling conditions to form a sulfinamide 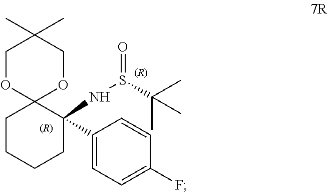
(c) reacting the sulfinamide 7R with acid to form the corresponding amine 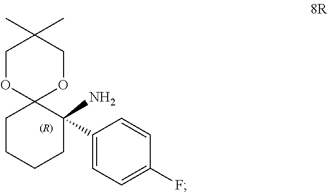
(d) reacting the amine 8R with a deuterated acylating agent of formula DCOOD or deuterated acid derivative thereof under amide forming conditions to form the amide 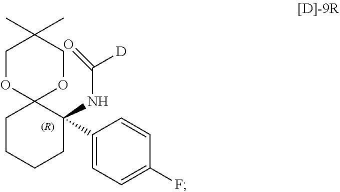
(e) reducing the acyl group in [D]-9R with a deuterated acyl reducing agent under acyl reducing conditions to form the compound 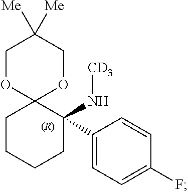
and
[D₃]-10R
(f) deprotecting the ketal in [D₃]-10R in the presence of acid, then treating the resulting product with base to form the compound [D₃]-11R.

81. The process according to claim 56 for preparing a compound of the formula: 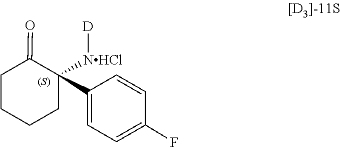
which process comprises
(a) reacting a compound having a ketal protecting group of the structure 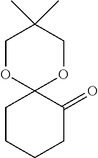
with a sulfinamide of the structure 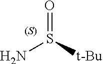
in the presence of Ti(OEt)₄ to form an imine 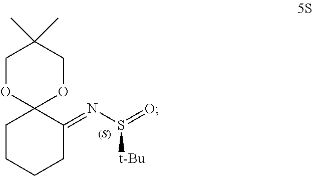
(b) reacting the imine 5S with an organometallic compound of the structure 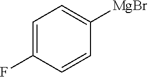
under Grignard coupling conditions to form a sulfinamide 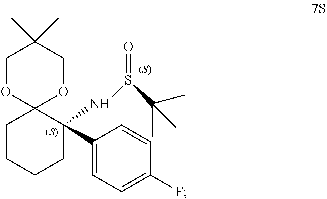
(c) reacting the sulfinamide 7S with acid to form the corresponding amine 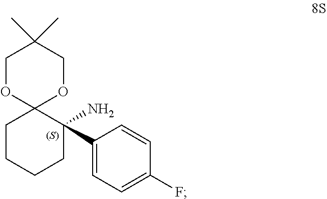
(d) reacting the amine 8S with an acylating agent of formula DCOOD or deuterated acid derivative thereof under amide forming conditions to form the amide 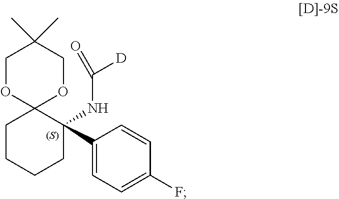
(e) reducing the acyl group in [D]-9S with a deuterated acyl reducing agent under acyl reducing conditions to form the compound 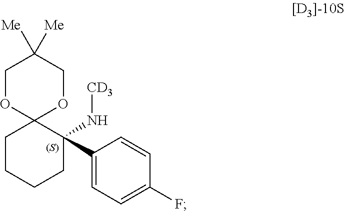
and
(f) deprotecting the ketal in [D₃]-10S in the presence of acid, then treating the resulting product with base to form the compound [D₃]-11S.

82. The process according to claim 79 wherein the deuterated acyl reducing agent is NaBD₄ or LiAlD₄.

83. The process according to claim 79, wherein 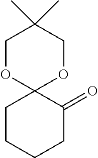
is prepared by reacting 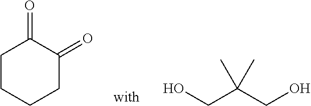
in the presence of acid.

84. The process according to claim 56 wherein the deprotecting step is conducted in concentrated hydrochloric acid in water.

85. The process according to claim 80 wherein the deuterated acyl reducing agent is NaBD₄ or LiAlD₄.

86. The process according to claim 81 wherein the deuterated acyl reducing agent is NaBD₄ or LiAlD₄.

87. The process according to claim 80, wherein 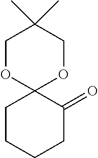
is prepared by reacting 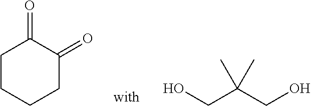
in the presence of acid.

88. The process according to claim 81, wherein 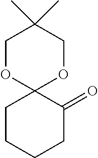
is prepared by reacting 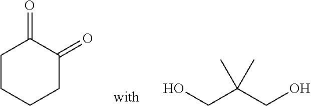
in the presence of acid.
